(19) United States
(12) Patent Application Publication
(10) Pub. No.: US 2015/0160225 A1
(43) Pub. Date: Jun. 11, 2015

(54) Renal Cell Carcinoma Biomarkers and Uses Thereof

(71) Applicant: SomaLogic, Inc., Boulder, CO (US)

(72) Inventors: Rachel M. Ostroff, Westminster, CO (US); Stephen Alaric Williams, Boulder, CO (US); Michael Riel-Mehan, Louisville, CO (US); Edward N. Brody, Boulder, CO (US); Alex A.E. Stewart, Waltham, MA (US); Shintaro Kato, Boulder, CO (US)

(21) Appl. No.: 14/566,431

(22) Filed: Dec. 10, 2014

Related U.S. Application Data
(63) Continuation of application No. 13/592,267, filed on Aug. 22, 2012, now abandoned.
(60) Provisional application No. 61/526,133, filed on Aug. 22, 2011.

(51) Int. Cl. G01N 33/574; G06F 19/20

(52) U.S. Cl. CPC G01N 33/57438; G06F 19/20; G01N 2333/96433; G01N 2333/96494; G01N 2333/8121; G01N 2333/805; G01N 2333/521; G01N 2333/70503; G01N 2333/4706; G01N 2333/4703; G01N 2333/78

(57) ABSTRACT

The subject disclosure concerns methods for evaluation of renal cell carcinoma (RCC). Such methods include a method of determining of a diagnosis of the individual as having or not having RCC; determination of a prognosis of a future course of RCC; determination of disease burden; or determination of recurrence of RCC in an individual who had been apparently cured of RCC. The methods each involve the detection of the value of at least one biomarker of Table 1. The biomarker value is used, in some of the methods, to determine whether the individual does or does not demonstrate evidence of disease, and in another method, to determine the degree or a score indicative of the individual's extent of disease.

### RELATED APPLICATIONS

This application is a continuation application of U.S. application Ser. No. 13/592,267, filed Aug. 22, 2012, which claims the benefit of U.S. Provisional Application Ser. No. 61/526,133, filed Aug. 22, 2011, each of which is incorporated herein by reference in its entirety.

### FIELD OF THE INVENTION

The present invention relates generally to the detection of biomarkers for Renal Cell Carcinoma (RCC) in an individual and, more specifically, to one or more biomarkers, methods, devices, reagents, systems, and kits for the evaluation of RCC, wherein the evaluation may comprise diagnosis, prognosis, determination of disease burden or determination of recurrence of RCC in an individual.

### BACKGROUND

The following description provides a summary of information relevant to the present disclosure and is not an admission that any of the information provided or publications referenced herein is prior art to the present disclosure.

Approximately 70,000 people/year present in the US with suspicious renal mass and this number is expected to climb as abdominal imaging rates increase. In 2010, approximately 58,000 people will be diagnosed and about 13,000 will die from RCC in the United States. While most renal masses are found to be simple cysts, a significant number show contrast enhancement and are therefore suggestive of cancer. Eighty percent of non-cystic lesions are malignant, yet most are slow growing. Deciding among the options of surveillance and surgical excision, especially of small masses less than 4 cm in diameter and especially in patients with comorbidities, is often difficult. A prognostic risk assessment tool would enable the physician to consider individual treatment options.

Based on incidence and mortality rates, it is estimated that the prevalence of diagnosed RCC in the US is 250,000 people. Prognosis and periodic monitoring for recurrence are significant clinical opportunities. Approximately 25% of cases are diagnosed with metastatic or loco-regional advanced disease, and are at risk for recurrence. Prognosis is correlated with stage and histological grade at diagnosis, and the most useful blood prognostic markers would add predictive information that is complementary to pathology. Negative prognostic signs include a poor performance status, the presence of symptoms and/or paraneoplastic syndromes (e.g., anemia, hypercalcemia, hepatopathy, thrombocytosis, fever, weight loss), and obesity.

Surgery may be curative when patients diagnosed with RCC first present with localized disease. However, many patients who are initially resected eventually relapse, and the prognosis in these cases is poor. Local recurrence occurs in about 5% of patients. It is associated with incomplete resection of the primary tumor, positive surgical margins, and regional lymph node metastasis. Distant metastases are present at the time of diagnosis in up to 30% of patients. Among those with localized disease who are treated surgically, 20-30% will eventually develop distant metastasis. In addition, 3% of RCC patients present with a second primary tumor.

Early diagnosis of patients with isolated local recurrence is important because surgical resection of such relapses may improve outcome. Thus, optimal management requires careful surveillance for recurrent disease in those who have undergone a potentially curative resection, particularly in the first 3-5 years post-surgery. The most common sites of metastatic disease from RCC are the lungs, bones, liver, renal fossa, and brain. Laboratory tests of liver function, LDH, serum calcium and alkaline phosphatase are routinely done to monitor for metastasis. A blood test that detects recurrence prior to radiological or clinical presentation would allow for rapid treatment decisions that may limit the extent of recurrent disease.

Biomarker selection for a specific disease state involves first the identification of markers that have a measurable and statistically significant difference in a disease population compared to a control population for a specific medical application. Biomarkers can include secreted or shed molecules that parallel disease development or progression and readily diffuse into the blood stream from RCC tissue or from surrounding tissues and circulating cells in response to a RCC. The biomarker or set of biomarkers identified are generally clinically validated or shown to be a reliable indicator for the original intended use for which it was selected. Biomarkers can include small molecules, peptides, proteins, and nucleic acids. Some of the key issues that affect the identification of biomarkers include over-fitting of the available data and bias in the data.

A variety of methods have been utilized in an attempt to identify biomarkers for evaluation, diagnosis, prognosis and determination of recurrence of disease. For protein-based markers, these include two-dimensional electrophoresis, mass spectrometry, and immunoassay methods. For nucleic acid markers, these include mRNA expression profiles, microRNA profiles, FISH, serial analysis of gene expression (SAGE), methylation profiles, and large scale gene expression arrays.

The utility of two-dimensional electrophoresis is limited by low detection sensitivity; issues with protein solubility, charge, and hydrophobicity; gel reproducibility; and the possibility of a single spot representing multiple proteins. For mass spectrometry, depending on the format used, limitations revolve around the sample processing and separation, sensitivity to low abundance proteins, signal to noise considerations, and inability to immediately identify the detected protein. Limitations in immunoassay approaches to biomarker discovery are centered on the inability of antibody-based multiplex assays to measure a large number of analytes. One might simply print an array of high-quality antibodies and, without sandwiches, measure the analytes bound to those antibodies. (This would be the formal equivalent of using a whole genome of nucleic acid sequences to measure by hybridization all DNA or RNA sequences in an organism or a cell. The hybridization experiment works because hybridization can be a stringent test for identity. Even very good antibodies are not stringent enough in selecting their binding partners to work in the context of blood or even cell extracts because the protein ensemble in those matrices have extremely different abundances.) Thus, one must use a different approach with immunoassay-based approaches to biomarker discovery—one would need to use multiplexed ELISA assays (that is, sandwiches) to get sufficient stringency to measure many analytes simultaneously to decide which analytes are indeed biomarkers. Sandwich immunoassays do not scale to high content, and thus biomarker discovery using stringent sandwich immunoassays is not possible using standard array formats. Lastly, antibody reagents are subject to substantial lot variability and reagent instability. The instant platform for protein biomarker discovery overcomes this problem.

Many of these methods rely on or require some type of sample fractionation prior to the analysis. Thus, the sample preparation required to run a sufficiently powered study designed to identify and discover statistically relevant biomarkers in a series of well-defined sample populations is extremely difficult, costly, and time consuming. During fractionation, a wide range of variability can be introduced into the various samples. For example, a potential marker could be unstable to the process, the concentration of the marker could be changed, inappropriate aggregation or disaggregation could occur, and inadvertent sample contamination could occur and thus obscure the subtle changes anticipated in early disease.

It is widely accepted that biomarker discovery and detection methods using these technologies have serious limitations for the identification of diagnostic biomarkers. These limitations include an inability to detect low-abundance biomarkers, an inability to consistently cover the entire dynamic range of the proteome, irreproducibility in sample processing and fractionation, and overall irreproducibility and lack of robustness of the method. Further, these studies have introduced biases into the data and not adequately addressed the complexity of the sample populations, including appropriate controls, in terms of the distribution and randomization required to identify and validate biomarkers within a target disease population.

Although efforts aimed at the discovery of new and effective biomarkers have gone on for several decades, the efforts have been largely unsuccessful. Biomarkers for various diseases typically have been identified in academic laboratories, usually through an accidental discovery while doing basic research on some disease process. Based on the discovery and with small amounts of clinical data, papers were published that suggested the identification of a new biomarker. Most of these proposed biomarkers, however, have not been confirmed as real or useful biomarkers, primarily because the small number of clinical samples tested provide only weak statistical proof that an effective biomarker has in fact been found. That is, the initial identification was not rigorous with respect to the basic elements of statistics. In each of the years 1994 through 2003, a search of the scientific literature shows that thousands of references directed to biomarkers were published. During that same time frame, however, the FDA approved for diagnostic use, at most, three new protein biomarkers a year, and in several years no new protein biomarkers were approved.

Based on the history of failed biomarker discovery efforts, mathematical theories have been proposed that further promote the general understanding that biomarkers for disease are rare and difficult to find. Biomarker research based on 2D gels or mass spectrometry supports these notions. Very few useful biomarkers have been identified through these approaches. However, it is usually overlooked that 2D gel and mass spectrometry measure proteins that are present in blood at approximately 1 nM concentrations and higher, and that this ensemble of proteins may well be the least likely to change with disease. Other than the instant biomarker discovery platform, proteomic biomarker discovery platforms that are able to accurately measure protein expression levels at much lower concentrations do not exist.

Much is known about biochemical pathways for complex human biology. Many biochemical pathways culminate in or are started by secreted proteins that work locally within the pathology, for example growth factors are secreted to stimulate the replication of other cells in the pathology, and other factors are secreted to ward off the immune system, and so on. While many of these secreted proteins work in a paracrine fashion, some operate distally in the body. One skilled in the art with a basic understanding of biochemical pathways would understand that many pathology-specific proteins ought to exist in blood at concentrations below (even far below) the detection limits of 2D gels and mass spectrometry. What must precede the identification of this relatively abundant number of disease biomarkers is a proteomic platform that can analyze proteins at concentrations below those detectable by 2D gels or mass spectrometry.

Accordingly, a need exists for biomarkers, methods, devices, reagents, systems, and kits that enable the diagnosis, prognosis, determination of disease burden and determination of recurrence of RCC.

### SUMMARY

The present disclosure includes biomarkers, methods, reagents, devices, systems, and kits for the pre- and/or post-surgical evaluation of RCC. The biomarkers of the present disclosure were identified using a multiplex aptamer-based assay, which is described in detail in Example 1. By using the aptamer-based biomarker identification method described herein, this application describes a surprisingly large number of RCC biomarkers that are useful for the pre- and/or post-surgical evaluation of RCC. In identifying these biomarkers, about 1030 proteins from hundreds of individual samples were measured, some of which were at concentrations in the low femtomolar range. This is about four orders of magnitude lower than biomarker discovery experiments done with 2D gels or mass spectrometry.

While certain of the described RCC biomarkers are useful for the pre- and/or post-surgical evaluation of RCC, methods are described herein for the grouping of multiple subsets of the RCC biomarkers that are useful as a panel of biomarkers. Once an individual biomarker or subset of biomarkers has been identified, the pre- and/or post-surgical evaluation of RCC in an individual can be accomplished using any assay platform or format that is capable of measuring differences in the levels of the selected biomarker or biomarkers in a biological sample.

However, it was only by using the multiplex aptamer-based biomarker identification method described herein, wherein about 1030 separate potential biomarker values were individually screened from a large number of individuals who were postoperatively diagnosed as either having or not having RCC and clinical outcome determined through follow-up, that it was possible to identify the RCC evaluation biomarkers of Table 1. This discovery approach is in stark contrast to biomarker discovery using conditioned media or lysed cells as it queries a more patient-relevant system that requires no translation to human pathology.

Thus, in one aspect of the instant application, one or more biomarkers are provided for use either alone or in various combinations for diagnosis, prognosis, determination of RCC disease burden or determination of recurrence of RCC in an individual. Exemplary embodiments include the biomarkers provided in Table 1, which as noted above, were identified using a multiplex aptamer-based assay, as described generally in Example 1 and more specifically in Example 3. The markers provided in Table 1 are useful in diagnosing RCC, providing prognosis data in pre-surgical blood specimens, indicating disease burden and determining recurrence of RCC.

While certain of the described RCC biomarkers are useful alone for the pre- and/or post-surgical evaluation of RCC, methods are also described herein for the grouping of multiple subsets of the RCC biomarkers that are each useful as a panel of two or more biomarkers. Thus, various embodiments of the instant application provide combinations comprising N biomarkers, wherein N is at least two biomarkers. In other embodiments, N is selected to be any number from 2-48 biomarkers.

In yet other embodiments, N is selected to be any number from 2-5, 2-10, 2-15, 2-20, 2-25, 2-30, 2-35, 2-40, 2-45, or 2-48. In other embodiments, N is selected to be any number from 3-5, 3-10, 3-15, 3-20, 3-25, 3-30, 3-35, 3-40, 3-45, or 3-48. In other embodiments, N is selected to be any number from 4-5, 4-10, 4-15, 4-20, 4-25, 4-30, 4-35, 4-40, 4-45, or 4-48. In other embodiments, N is selected to be any number from 5-10, 5-15, 5-20, 5-25, 5-30, 5-35, 5-40, 5-45, or 5-48. In other embodiments, N is selected to be any number from 6-10, 6-15, 6-20, 6-25, 6-30, 6-35, 6-40, 6-45, or 6-48. In other embodiments, N is selected to be any number from 7-10, 7-15, 7-20, 7-25, 7-30, 7-35, 7-40, 7-45, or 7-48. In other embodiments, N is selected to be any number from 8-10, 8-15, 8-20, 8-25, 8-30, 8-35, 8-40, 8-45, or 8-48. In other embodiments, N is selected to be any number from 9-10, 9-15, 9-20, 9-25, 9-30, 9-35, 9-40, 9-45, or 9-48. In other embodiments, N is selected to be any number from 10-15, 10-20, 10-25, 10-30, 10-35, 10-40, 10-45, or 10-48. It will be appreciated that N can be selected to encompass similar, but higher order, ranges.

In another aspect, a method is provided for evaluating RCC in an individual, the method including detecting, in a biological sample from an individual, at least one biomarker value corresponding to at least one biomarker selected from the group of biomarkers provided in Table 1, wherein the individual is classified for diagnosis, prognosis, determination of disease burden or determination of recurrence of RCC based on the at least one biomarker value.

In another aspect, a method is provided for evaluating RCC in an individual, the method including detecting, in a biological sample from an individual, biomarker values that each correspond to one of at least N biomarkers selected from the group of biomarkers set forth in Table 1, wherein the likelihood of the individual having RCC, having a poor prognosis or recurrence, or having an increased disease burden, is determined based on the biomarker values.

In another aspect, a method is provided for evaluating RCC in an individual, the method including detecting, in a biological sample from an individual, biomarker values that each correspond to one of at least N biomarkers selected from the group of biomarkers set forth in Table 1, wherein the individual is classified for diagnosis, prognosis, determination of disease burden or determination of recurrence of RCC based on the biomarker values.

Evaluation of RCC, as used herein, refers to evaluating whether an individual has a first evaluation of no evidence of disease (NED) when at least one biomarker of Table 1 is not detected as differentially expressed from the control distribution, or has a second evaluation of evidence of disease (EVD) when at least one biomarker of Table 1 is detected as differentially expressed from the control distribution.

In another aspect, a method is provided for evaluating an individual for RCC, wherein the evaluating comprises a determination of a diagnosis of the individual as having or not having RCC, determination of a prognosis of a future course of the RCC, determining disease burden, recurrence of RCC in an individual who had been apparently cured of the RCC, or any combination thereof. The evaluating can be conducted pre-surgically or post-surgically.

The evaluation of the individual for RCC includes detecting in the individual's biological sample, biomarker values of at least one biomarker of Table 1 or of a panel of biomarkers selected from Table 1. The panel comprises at least two biomarkers.

The number of biomarkers, N, selected from Table 1, can be any number described herein. In several embodiments, N is selected from the following ranges: N=1-10, N=2 to 10, N=3 to 10, N=4 to 10 and N=5 to 10. In another embodiment, the biomarker or biomarker panel comprises at least one of the following biomarkers: STC1, CXCL13 and MMP7. In other aspects, the panel can comprise at least all of STC1, CXCL13 and MMP7, or can comprise at least CXCL13 or at least STC1.

The biomarker panel can include, in addition to the at least one biomarker of Table 1, biomarkers not found in Table 1.

The method of evaluating an individual for RCC can combine the detection of biomarkers with the input of additional biomedical information. Such additional information is described in detail herein. The evaluating of the individual for RCC can further include, in addition to the detection of biomarkers, the imaging of the individual using the biomarkers of Table 1 that have been detectably labeled. The evaluation of the individual can include the use of the biomarker detection information and other foregoing information in the selection of a treatment option.

In another embodiment, the evaluating comprises determining a diagnoses of an individual by detecting a biomarker value corresponding to an at least one biomarker of Table 1 in a biological sample of the individual. The determination of diagnosis comprises a determination of no evidence of disease (NED) and no RCC when there is substantially no differential expression of the biomarker value of the individual relative to a biomarker value of the control population, or a diagnosis of evidence of disease (EVD) and RCC when there is a substantial differential expression of the biomarker value of the individual relative to the biomarker value of the control population. The diagnosis can be for any stage of RCC, or may comprise a diagnosis of any or all of Stages I-IV or II-IV of the RCC.

In one aspect, the method of determining a diagnosis comprises assaying a biological sample of an individual to detect a biomarker value corresponding to at least one biomarker of Table 1, comparing the biomarker value of the individual to a biomarker value of a control population to determine whether there is a differential expression; and classifying the individual as not having or having a diagnosis of RCC, where there is, respectively, no differential expression relative to the control population (no RCC), or with the diagnosis of RCC where there is a differential expression relative to the control population.

In another aspect, the evaluating of RCC comprises determining a prognosis by detecting no evidence of disease (NED) and a prediction of no RCC, or determining evidence of disease (EVD) and a prognosis of RCC.

In one aspect, the determining of a prognosis method can comprise assaying a biological sample of an individual to determine a biomarker value corresponding to at least one biomarker of Table 1, comparing the biomarker value of the individual to a biomarker value of a control population to determine if there is a differential expression; and classifying the individual as having no differential expression and a negative prediction for RCC at a defined time point in the future; or as having a differential expression and a prognosis for RCC at a defined time point in the future. The determination of prognosis can be helpful in evaluating an RCC patient and in selecting an appropriate therapy or surgery.

In another aspect, a method of evaluating is provided that comprises determining the disease burden of RCC in an individual. This method includes selecting a RCC disease burden vector (DBV) modeled on biomarkers that correlate with RCC stage; providing an individual's sample suspected of containing said biomarkers; applying the DBV to the sample biomarkers to determine the individual's disease burden vector score (DBV score); and determining the disease burden on the basis of the DBV score.

In another aspect, a method of evaluating is provided that comprises determining the recurrence of RCC in an individual who had apparently been cured of RCC, wherein the determining of recurrence comprises a first determination of no evidence of disease (NED) or a second determination of evidence of disease (EVD). The first determination of NED indicates no recurrence of RCC, and the second determination of EVD indicates recurrence of the RCC.

The method of determining recurrence can comprise assaying a biological sample of an individual to determine a biomarker value corresponding to an at least one biomarker of Table 1, comparing the biomarker value of the individual to a biomarker value of a control population to determine if there is differential expression, and classifying the individual as having said first determination of no RCC recurrence when there is no differential expression relative to the control population, or said second determination of RCC recurrence when there is differential expression relative to the control value.

The foregoing determination of recurrence of RCC can be repeated periodically with the patient in order to monitor the patient's progress following surgery or therapy, or during the course of therapy. The monitoring of recurrence of RCC can be useful in selecting a treatment option for the patient.

In another aspect, a classifier is provided, wherein the classifier comprises at least one, and preferably at least two biomarkers of Table 1. The biomarkers are selected on the basis of specificity and sensitivity in classifying unknown or case samples into the correct categories of NED or EVD. Selection of appropriate biomarkers to obtain acceptable specificity and sensitivity are described herein in detail.

In another aspect, a computer-implemented method is provided for classifying an individual as either having a first evaluation of NED, or as having a second evaluation of EVD. This method can comprise retrieving on a computer biomarker information for an individual, wherein the biomarker information comprises a biomarker value that corresponds to the at least one biomarker of Table 1, comparing said biomarker value of step a) to a biomarker value of a control population to determine if there is differential expression, and classifying the individual as having a first evaluation of NED when there is no differential expression of the biomarker value of the individual relative to the control population, or has having a second evaluation of EVD when there is differential expression of the biomarker value of the individual relative to the control population.

In the computer-implemented method, the evaluation can comprise a diagnosis, prognosis, determination of disease burden, determination of recurrence of RCC, and/or a combination thereof. The evaluation of NED can be indicative of a diagnosis of no RCC, a prognosis of an outcome of no RCC at a selected future time point, a determination of no recurrence of RCC, and/or a combination thereof. The evaluation of EVD can be indicative of a diagnosis of the presence of RCC, a prognosis of an outcome of RCC at a selected future time point, a determination of recurrence of RCC, and/or a combination thereof.

In another aspect, a computer program product includes a computer readable medium embodying program code executable by a processor of a computing device or system, the program code comprising: code that retrieves data attributed to a biological sample from an individual, wherein the data comprises biomarker values that correspond to at least one of the biomarkers provided in Table 1; code for comparing the biomarker value of the individual to a biomarker value of a control population; and code that executes a classification method that indicates a first evaluation of NED when there is no differential expression of the individual's biomarker value relative to the control population, or a second evaluation of EVD when there is differential expression of the individual's biomarker value relative to the control population.

In another aspect, the computer-implemented classification of RCC status of an individual by the computer program product or the computer readable medium can reflect a diagnosis, prognosis, determination of disease burden, determination of recurrence of RCC, and/or a combination thereof. The evaluation of NED can be indicative of a diagnosis classification of no RCC, a prognosis classification of an outcome of no RCC at a selected future time point, a determination classification of no recurrence of RCC, and/or a combination thereof. The evaluation of EVD can be indicative of a diagnosis classification of RCC, a prognosis classification of an outcome of RCC at a selected future time point, a determination classification of recurrence of RCC, and/or a combination thereof.

### BRIEF DESCRIPTION OF THE DRAWINGS

FIG. 1A is a flowchart for an exemplary method for evaluating RCC in a biological sample.

FIG. 1B is a flowchart for an exemplary method for evaluating RCC in a biological sample using a naïve Bayes classification method.

FIG. 2 illustrates an exemplary computer system for use with various computer-implemented methods described herein.

FIG. 3 is a flowchart for a method of indicating the likelihood that an individual has RCC in accordance with one embodiment.

FIG. 4 is a flowchart for a method of indicating the likelihood that an individual has RCC in accordance with one embodiment.

FIG. 5 illustrates an exemplary aptamer assay that can be used to detect one or more RCC biomarkers in a biological sample.

FIG. 6 shows box plots of 10 SOMAmers in the random forest (RF) Outcome model. Control is NED, Disease is EVD. Y-axis is SOMAmer assay RFU.

FIG. 7 shows ROC curves for the RF Outcome model training set and testing the model bases on pathologic stage.

FIG. 8 shows ROC curves for the RF Outcome model training set and testing the model based on TP2 Outcome.

FIG. 9 shows box plots of the distribution of SOMAmer measurements with pathologic state. “None” is from BEN (non-malignant) subjects. Y-axis is SOMAmer assay RFU.

FIG. 10 shows box plots of SOMAmer signals from control subjects with BEN or NED compared to cases who were Never Disease Free (NDF) or who had RCC recurrence. The numbers on the x-axis are days from TP1 blood collection to recurrence. Y-axis is SOMAmer assay RFU.

FIG. 11 shows ROC curves for the RF Outcome model training set and testing with the blinded TP1 Outcome verification set.

FIG. 12 shows box plots of the distribution of the biomarkers in the RF Diagnosis model by RCC stage.

FIG. 13 shows the ROC curve for the RF Diagnosis model classifier for distinguishing BEN (benign) from stages II-IV RCC.

FIG. 14 shows the DBV constructed with markers from the SGPLS analysis. “0” indicates benign renal condition.

FIG. 15 shows the DBV constructed with markers from the LASSO analysis. “0” indicates benign renal condition.

FIG. 16 shows a ROC curve for a single biomarker, STC1, using a naïve Bayes classifier for a test that detects RCC Outcome.

FIG. 17 shows ROC curves for biomarker panels of from two to ten biomarkers using naïve Bayes classifiers for a test that detects RCC Outcome.

FIG. 18 illustrates the change in the classification score (AUC) as the number of biomarkers is increased from one to ten using naïve Bayes classification for an RCC Outcome panel.

FIG. 19 shows a histogram of frequencies for which biomarkers were used in building classifiers to distinguish between EVD and NED individuals from an aggregated set of potential biomarkers.

FIG. 20 shows the measured biomarker distributions for STC1 as a cumulative distribution function (cdf) in log-transformed RFU for the NED control group (solid line) and the EVD disease group (dotted line) along with their curve fits to a normal cdf (dashed lines) used to train the naïve Bayes classifiers.

FIG. 21A shows a pair of histograms summarizing all possible single protein naïve Bayes classifier scores (AUC) using the biomarkers set forth in Table 1 (white) and a set of random markers (black).

FIG. 21B shows a pair of histograms summarizing all possible two-protein protein naïve Bayes classifier scores (AUC) using the biomarkers set forth in Table 1 (white) and a set of random markers (black).

FIG. 21C shows a pair of histograms summarizing all possible three-protein naïve Bayes classifier scores (AUC) using the biomarkers set forth in Table 1 (white) and a set of random markers (black).

FIG. 22 shows the AUC for naïve Bayes classifiers using from 2-10 markers selected from the full panel and the scores obtained by dropping the best 5, 10, and 15 markers during classifier generation.

FIG. 23A shows a set of ROC curves modeled from the data in Table 15 for panels of from two to five markers.

FIG. 23B shows a set of ROC curves computed from the training data for panels of from two to five markers as in FIG. 22A.

### DETAILED DESCRIPTION

Reference will now be made in detail to representative embodiments of the invention. While the invention will be described in conjunction with the enumerated embodiments, it will be understood that the invention is not intended to be limited to those embodiments. On the contrary, the invention is intended to cover all alternatives, modifications, and equivalents that may be included within the scope of the present invention as defined by the claims.

One skilled in the art will recognize many methods and materials similar or equivalent to those described herein, which could be used in and are within the scope of the practice of the present invention. The present invention is in no way limited to the methods and materials described.

Unless defined otherwise, technical and scientific terms used herein have the same meaning as commonly understood by one of ordinary skill in the art to which this invention belongs. Although any methods, devices, and materials similar or equivalent to those described herein can be used in the practice or testing of the invention, the preferred methods, devices and materials are now described.

All publications, published patent documents, and patent applications cited in this application are indicative of the level of skill in the art(s) to which the application pertains. All publications, published patent documents, and patent applications cited herein are hereby incorporated by reference to the same extent as though each individual publication, published patent document, or patent application was specifically and individually indicated as being incorporated by reference.

As used in this application, including the appended claims, the singular forms “a,” “an,” and “the” include plural references, unless the content clearly dictates otherwise, and are used interchangeably with “at least one” and “one or more.” Thus, reference to “an aptamer” includes mixtures of aptamers, reference to “a probe” includes mixtures of probes, and the like.

As used herein, the term “about” represents an insignificant modification or variation of the numerical value such that the basic function of the item to which the numerical value relates is unchanged.

As used herein, the terms “comprises,” “comprising,” “includes,” “including,” “contains,” “containing,” and any variations thereof, are intended to cover a non-exclusive inclusion, such that a process, method, product-by-process, or composition of matter that comprises, includes, or contains an element or list of elements does not include only those elements but may include other elements not expressly listed or inherent to such process, method, product-by-process, or composition of matter.

The present application includes biomarkers, methods, devices, reagents, systems, and kits for the evaluation of RCC in an individual. Such evaluation can be conducted pre-surgically or post-surgically. The specific intended uses and clinical applications for the subject invention include: 1) diagnosis of the presence or absence of RCC; 2) prognosis of the outcome of RCC in an individual at a selected future time point; 3) determination of disease burden and 4) monitoring of recurrence of RCC in an individual that has apparently been cured of RCC.

In one aspect, one or more biomarkers are provided for use either alone or in various combinations to evaluate RCC, including the diagnosis of RCC in an individual, the prognosis of the outcome of RCC, the determination of disease burden, the monitoring of recurrence of RCC, or the addressing other clinical indications. As described in detail below, exemplary embodiments include the biomarkers provided in Table 1, which were identified using a multiplex aptamer-based assay, as described generally in Example 1 and more specifically in Example 3, and according to the method of Gold L. et al. (2010) Aptamer-Based Multiplexed Proteomic Technology for Biomarker Discovery. PLoS ONE 5(12):e15004. doi:10.1371/journal.pone.0015004.

Table 1 sets forth the findings obtained from analyzing blood samples from 173 individuals diagnosed with RCC. The training group was designed to match the population with which a prognostic RCC diagnostic test can have significant benefit. These cases and controls were obtained from a single clinical site.

The potential biomarkers were measured in individual samples rather than pooling the disease and control blood; this allowed a better understanding of the individual and group variations in the phenotypes associated with the presence and absence of disease (in this case RCC). Since about 1030 protein measurements were made on each sample, and a total of 385 samples from both the disease and the control populations were individually measured, Table 1 resulted from an analysis of an uncommonly large set of data. The measurements were analyzed using the methods described in the section, “Classification of Biomarkers and Calculation of RCC Prognosis Scores” herein. Table 1 lists the 48 biomarkers found to be useful in evaluating RCC status, such as prognosis, diagnosis, recurrence, or disease burden, in samples obtained from individuals with RCC or an outcome of EVD from “control” samples obtained from individuals without benign renal conditions, or RCC patients determined to have a NED outcome.

While certain of the described RCC biomarkers are useful alone for diagnosing, prognosing, determining disease burden and/or determining the recurrence of RCC, methods are also described herein for the grouping of multiple subsets of the biomarkers, where each grouping or subset selection is useful as a panel of two or more biomarkers, interchangeably referred to herein as a “biomarker panel” and a panel. Thus, various embodiments of the instant application provide combinations comprising N biomarkers, wherein N is at least two biomarkers. In other embodiments, N is selected from 2-48 biomarkers.

In yet other embodiments, N is selected to be any number from 2-5, 2-10, 2-15, 2-20, 2-25, 2-30, 2-35, 2-40, 2-45, or 2-48. In other embodiments, N is selected to be any number from 3-5, 3-10, 3-15, 3-20, 3-25, 3-30, 3-35, 3-40, 3-45, or 3-48. In other embodiments, N is selected to be any number from 4-5, 4-10, 4-15, 4-20, 4-25, 4-30, 4-35, 4-40, 4-45, or 4-48. In other embodiments, N is selected to be any number from 5-10, 5-15, 5-20, 5-25, 5-30, 5-35, 5-40, 5-45, or 5-48. In other embodiments, N is selected to be any number from 6-10, 6-15, 6-20, 6-25, 6-30, 6-35, 6-40, 6-45, or 6-48. In other embodiments, N is selected to be any number from 7-10, 7-15, 7-20, 7-25, 7-30, 7-35, 7-40, 7-45, or 7-48. In other embodiments, N is selected to be any number from 8-10, 8-15, 8-20, 8-25, 8-30, 8-35, 8-40, 8-45, or 8-48. In other embodiments, N is selected to be any number from 9-10, 9-15, 9-20, 9-25, 9-30, 9-35, 9-40, 9-45, or 9-48. In other embodiments, N is selected to be any number from 10-15, 10-20, 10-25, 10-30, 10-35, 10-40, 10-45, or 10-48. It will be appreciated that N can be selected to encompass similar, but higher order, ranges.

In one embodiment, the number of biomarkers useful for a biomarker subset or panel is based on the sensitivity and specificity value for the particular combination of biomarker values. The terms “sensitivity” and “specificity” are used herein with respect to the ability to correctly classify the RCC diagnosis, RCC prognosis, RCC disease burden and RCC recurrence after apparent cure for an individual, based on one or more biomarker values detected in their biological sample. “Sensitivity” indicates the performance of the biomarker(s) with respect to correctly classifying individuals that have a positive RCC diagnosis, a positive RCC prognosis (EVD), or a positive RCC recurrence after apparent cure, i.e., evidence of disease (EVD). “Specificity” indicates the performance of the biomarker(s) with respect to correctly classifying individuals who have a negative RCC diagnosis, a negative RCC prognosis (NED), or a negative RCC recurrence following apparent cure of RCC. For example, 85% specificity and 90% sensitivity for a panel of markers used to test a set of control samples and RCC diagnosis samples indicates that 85% of the control samples were correctly classified as NED samples by the panel, and 90% of the positive samples were correctly classified as EVD samples by the panel. The desired or preferred minimum value of biomarkers can be determined as described in Example 11. The performance characteristics of representative panels are set forth in Table 18, which describes the results for series of 1000 different panels of 1-10 biomarkers, which have the indicated range of AUC values for each series of panels.

In one aspect, RCC Outcome is detected or diagnosed in an individual by conducting an assay on a biological sample from the individual and detecting biomarker values that each correspond to at least one of the biomarkers STC1, CXCL13 or MMP7 and at least N additional biomarkers selected from the list of biomarkers in Table 1, wherein N equals 2, 3, 4, 5, 6, 7, 8, or 9. In a further aspect, RCC Outcome is detected or diagnosed in an individual by conducting an assay on a biological sample from the individual and detecting biomarker values that each correspond to the biomarkers STC1, CXCL13 or MMP7 and one of at least N additional biomarkers selected from the list of biomarkers in Table 1, wherein N equals 1, 2, 3, 4, 5, 6, or 7. In a further aspect, RCC Outcome is detected or diagnosed in an individual by conducting an assay on a biological sample from the individual and detecting biomarker values that each correspond to the biomarker STC1 and one of at least N additional biomarkers selected from the list of biomarkers in Table 1, wherein N equals 2, 3, 4, 5, 6, 7, 8, or 9. In a further aspect, RCC Outcome is detected or diagnosed in an individual by conducting an assay on a biological sample from the individual and detecting biomarker values that each correspond to the biomarker CXCL13 and one of at least N additional biomarkers selected from the list of biomarkers in Table 1, wherein N equals 2, 3, 4, 5, 6, 7, 8, or 9. In a further aspect, RCC Outcome is detected or diagnosed in an individual by conducting an assay on a biological sample from the individual and detecting biomarker values that each correspond to the biomarker MMP7 and one of at least N additional biomarkers selected from the list of biomarkers in Table 1, wherein N equals 2, 3, 4, 5, 6, 7, 8, or 9.

The RCC biomarkers identified herein represent a considerable number of choices for subsets or panels of biomarkers that can be used to effectively evaluate an individual for RCC. Selection of the desired number of such biomarkers depends on the specific combination of biomarkers chosen. It is important to remember that panels of biomarkers for evaluation of RCC in an individual may also include biomarkers not found in Table 1, and that the inclusion of additional biomarkers not found in Table 1 may reduce the number of biomarkers in the particular subset or panel that is selected from Table 1. The number of biomarkers from Table 1 used in a subset or panel may also be reduced if additional biomedical information is used in conjunction with the biomarker values to establish acceptable sensitivity and specificity values for a given assay.

Another factor that can affect the number of biomarkers to be used in a subset or panel of biomarkers is the procedures used to obtain biological samples from individuals who are being evaluated for RCC. In a carefully controlled sample procurement environment, the number of biomarkers necessary to meet desired sensitivity and specificity values will be lower than in a situation where there can be more variation in sample collection, handling and storage. In developing the list of biomarkers set forth in Table 1, a single sample collection site was utilized to collect data for classifier training. Since samples were collected prior to clinical outcome, the study is free from case/control sample collection bias.

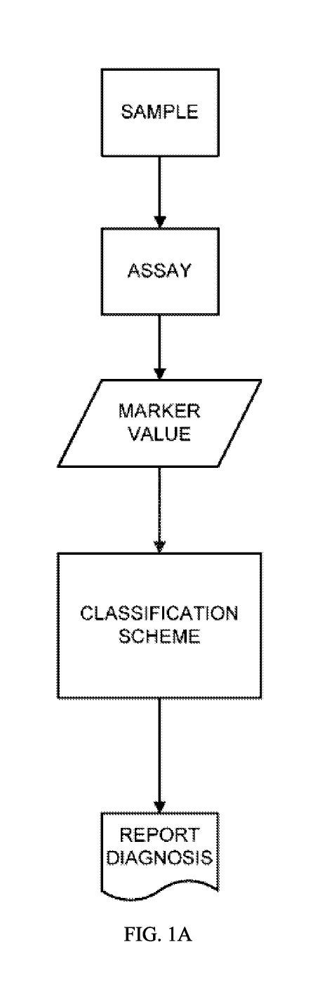

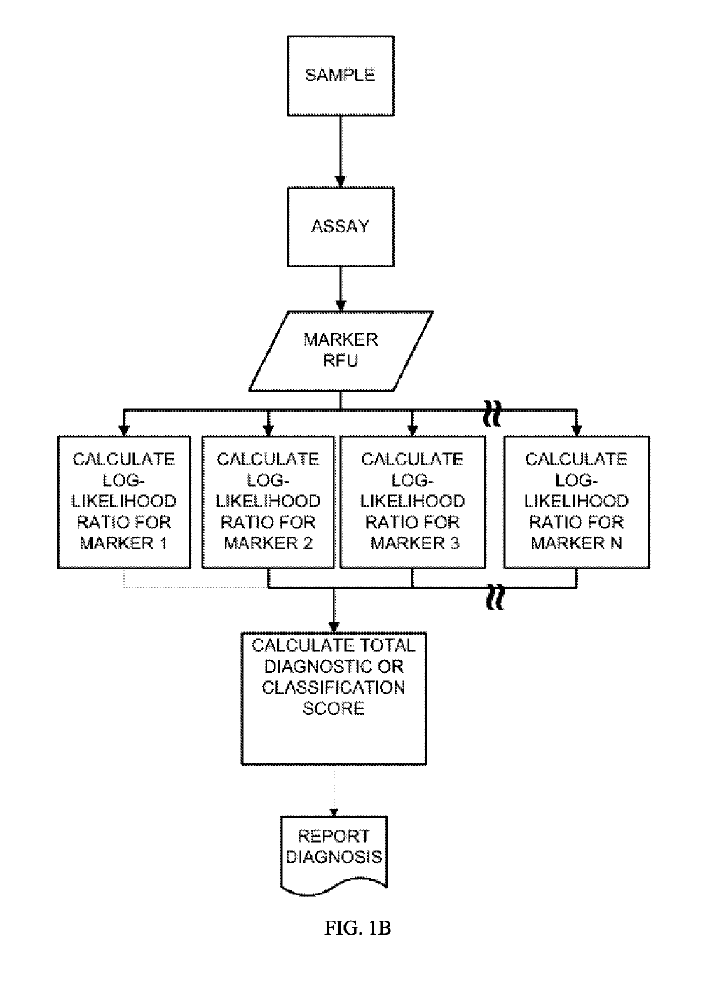

In one embodiment, the subject invention comprises obtaining a biological sample from an individual or individuals of interest. One example of the instant application can be described generally with reference to FIGS. 1A and 1B. The biological sample is assayed to detect the presence of one or more (N) biomarkers of interest and to determine a biomarker value for each of said N biomarkers (typically measured as marker RFU (relative fluorescence units)). Once a biomarker has been detected and a biomarker value assigned, each marker is scored or classified as described in detail herein. The marker scores are then combined to provide a total evaluation score, which reflects whether the individual has evidence of disease, i.e., current RCC diagnosis, prognosis of a future RCC outcome, extent of disease burden or current evidence of the recurrence of RCC after an apparent cure.

“Biological sample”, “sample”, and “test sample” are used interchangeably herein to refer to any material, biological fluid, tissue, or cell obtained or otherwise derived from an individual. This includes blood (including whole blood, leukocytes, peripheral blood mononuclear cells, buffy coat, plasma, and serum), sputum, tears, mucus, nasal washes, nasal aspirate, breath, urine, semen, saliva, cyst fluid, meningeal fluid, amniotic fluid, glandular fluid, lymph fluid, nipple aspirate, bronchial aspirate, pleural fluid, peritoneal fluid, synovial fluid, joint aspirate, ascites, cells, a cellular extract, and cerebrospinal fluid. This also includes experimentally separated fractions of all of the preceding. For example, a blood sample can be fractionated into serum or into fractions containing particular types of blood cells, such as red blood cells or white blood cells (leukocytes). If desired, a sample can be a combination of samples from an individual, such as a combination of a tissue and fluid sample. The term “biological sample” also includes materials containing homogenized solid material, such as from a stool sample, a tissue sample, or a tissue biopsy, for example. The term “biological sample” also includes materials derived from a tissue culture or a cell culture. Any suitable methods for obtaining a biological sample can be employed; exemplary methods include, e.g., phlebotomy, swab (e.g., buccal swab), lavage, fine needle aspirate biopsy procedure, and surgical excision. Samples can also be collected, e.g., by micro dissection (e.g., laser capture micro dissection (LCM) or laser micro dissection (LMD)), bladder wash, smear (e.g., a PAP smear), or ductal lavage. A “biological sample” obtained or derived from an individual includes any such sample that has been processed in any suitable manner after being obtained from the individual.

Further, it should be realized that a biological sample can be derived by taking biological samples from a number of individuals and pooling them or pooling an aliquot of each individual's biological sample. The pooled sample can be treated as a sample from a single individual and if the RCC evaluation indicates evidence of disease (EVD) in the pooled sample, then each individual biological sample can be re-tested to determine which individuals have EVD.

For purposes of this specification, the phrase “data attributed to a biological sample from an individual” is intended to mean that the data in some form derived from, or were generated using, the biological sample of the individual. The data may have been reformatted, revised, or mathematically altered to some degree after having been generated, such as by conversion from units in one measurement system to units in another measurement system; but, the data are understood to have been derived from, or were generated using, the biological sample.

“Target”, “target molecule”, and “analyte” are used interchangeably herein to refer to any molecule of interest that may be present in a biological sample. A “molecule of interest” includes any minor variation of a particular molecule, such as, in the case of a protein, for example, minor variations in amino acid sequence, disulfide bond formation, glycosylation, lipidation, acetylation, phosphorylation, or any other manipulation or modification, such as conjugation with a labeling component, which does not substantially alter the identity of the molecule. A “target molecule”, “target”, or “analyte” is a set of copies of one type or species of molecule or multi-molecular structure. “Target molecules”, “targets”, and “analytes” refer to more than one such set of molecules. Exemplary target molecules include proteins, polypeptides, nucleic acids, carbohydrates, lipids, polysaccharides, glycoproteins, hormones, receptors, methylated nucleic acid, antigens, antibodies, affybodies, antibody mimics, viruses, pathogens, toxic substances, substrates, metabolites, transition state analogs, cofactors, inhibitors, drugs, dyes, nutrients, growth factors, cells, tissues, and any fragment or portion of any of the foregoing.

As used herein, “polypeptide,” “peptide,” and “protein” are used interchangeably to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. Polypeptides can be single chains or associated chains. Also included within the definition are preproteins and intact mature proteins; peptides or polypeptides derived from a mature protein; fragments of a protein; splice variants; recombinant forms of a protein; protein variants with amino acid modifications, deletions, or substitutions; digests; and post-translational modifications, such as glycosylation, acetylation, phosphorylation, and the like.

As used herein, “marker” and “biomarker” are used interchangeably to refer to a target molecule that indicates or is a sign of a normal or abnormal process in an individual or of a disease or other condition in an individual. More specifically, a “marker” or “biomarker” is an anatomic, physiologic, biochemical, or molecular parameter associated with the presence of a specific physiological state or process, whether normal or abnormal, and, if abnormal, whether chronic or acute. Biomarkers are detectable and measurable by a variety of methods including laboratory assays and medical imaging. When a biomarker is a protein, it is also possible to use the expression of the corresponding gene as a surrogate measure of the amount or presence or absence of the corresponding protein biomarker in a biological sample or methylation state of the gene encoding the biomarker or proteins that control expression of the biomarker.

As used herein, “biomarker value”, “value”, “biomarker level”, and “level” are used interchangeably to refer to a measurement that is made using any analytical method for detecting the biomarker in a biological sample and that indicates the presence, absence, absolute amount or concentration, relative amount or concentration, titer, a level, an expression level, a ratio of measured levels, or the like, of, for, or corresponding to the biomarker in the biological sample. The exact nature of the “value” or “level” depends on the specific design and components of the particular analytical method employed to detect the biomarker.

When a biomarker indicates or is a sign of an abnormal process or a disease or other condition in an individual, that biomarker is generally described as being either over-expressed or under-expressed as compared to an expression level or value of the biomarker that indicates or is a sign of a normal process or an absence of a disease or other condition in an individual. “Up-regulation”, “up-regulated”, “over-expression”, “over-expressed”, and any variations thereof are used interchangeably to refer to a value or level of a biomarker in a biological sample that is greater than a value or level (or range of values or levels) of the biomarker that is typically detected in similar biological samples from healthy or normal individuals. The terms may also refer to a value or level of a biomarker in a biological sample that is greater than a value or level (or range of values or levels) of the biomarker that may be detected at a different stage of a particular disease.

“Down-regulation”, “down-regulated”, “under-expression”, “under-expressed”, and any variations thereof are used interchangeably to refer to a value or level of a biomarker in a biological sample that is less than a value or level (or range of values or levels) of the biomarker that is typically detected in similar biological samples from healthy or normal individuals. The terms may also refer to a value or level of a biomarker in a biological sample that is less than a value or level (or range of values or levels) of the biomarker that may be detected at a different stage of a particular disease.

Further, a biomarker that is either over-expressed or under-expressed can also be referred to as being “differentially expressed” or as having a “differential level” or “differential value” as compared to a “normal” or “control” expression level or value of the biomarker that indicates or is a sign of a normal or a control process or an absence of a disease or other condition in an individual. Thus, “differential expression” of a biomarker can also be referred to as a variation from a “normal” or “control” expression level of the biomarker.

The term “differential gene expression” and “differential expression” are used interchangeably to refer to a gene (or its corresponding protein expression product) whose expression is activated to a higher or lower level in a subject suffering from a specific disease, relative to its expression in a normal or control subject. The terms also include genes (or the corresponding protein expression products) whose expression is activated to a higher or lower level at different stages of the same disease. It is also understood that a differentially expressed gene may be either activated or inhibited at the nucleic acid level or protein level, or may be subject to alternative splicing to result in a different polypeptide product. Such differences may be evidenced by a variety of changes including mRNA levels, surface expression, secretion or other partitioning of a polypeptide. Differential gene expression may include a comparison of expression between two or more genes or their gene products; or a comparison of the ratios of the expression between two or more genes or their gene products; or even a comparison of two differently processed products of the same gene, which differ between normal subjects and subjects suffering from a disease; or between various stages of the same disease. Differential expression includes both quantitative, as well as qualitative, differences in the temporal or cellular expression pattern in a gene or its expression products among, for example, normal and diseased cells, or among cells which have undergone different disease events or disease stages.

As used herein, “individual” refers to a test subject or patient. The individual can be a mammal or a non-mammal. In various embodiments, the individual is a mammal. A mammalian individual can be a human or non-human. In various embodiments, the individual is a human. A healthy or normal individual is an individual in which the disease or condition of interest (including, for example, kidney diseases, renal mass-associated diseases, or other urinary tract conditions) is not detectable by conventional diagnostic methods.

“Diagnose”, “diagnosing”, “diagnosis”, and variations thereof refer to the detection, determination, or recognition of a health status or condition of an individual on the basis of one or more signs, symptoms, data, or other information pertaining to that individual. The health status of an individual can be diagnosed as healthy/normal (i.e., a diagnosis of the absence of a disease or condition) or diagnosed as ill/abnormal (i.e., a diagnosis of the presence, or an assessment of the characteristics, of a disease or condition). The terms “diagnose”, “diagnosing”, “diagnosis”, etc., encompass, with respect to a particular disease or condition, the initial detection of the disease; the characterization or classification of the disease; the detection of the progression, remission, or recurrence of the disease; the determination of disease burden; and the detection of disease response after the administration of a treatment or therapy to the individual. The diagnosis of RCC includes distinguishing individuals who have RCC from individuals who do not. It also includes diagnosis of any one or more of RCC Stages I-IV, and the differential diagnosis of Stages I-IV relative to a biological sample such as a benign renal mass. The phrase “determining diagnosis” can refer to the determination/detection of NED and the substantial absence of or no RCC, or the determination/detection of EVD and the diagnosis of RCC.

“Prognose”, “prognosing”, “prognosis”, and variations thereof refer to the prediction of a future course of a disease or condition in an individual who has the disease or condition (e.g., predicting patient survival), and such terms encompass the evaluation of disease response after surgery to remove a mass, or the administration of a treatment or therapy to the individual. “Prognosing” and variants thereof can also mean predicting evidence of disease (EVD) or no evidence of disease (NED) in the individual at a future preselected time point. The date of prognosing can be referred to as time point 1 (TP1), and the preselected future time point may be referred to as time point 2 (TP2) and can include a specific future date or range of dates, for example post-treatment follow-up. The phrase “determination of prognosis” can refer to the determination/detection of NED and a prediction of no recurrence of RCC at a predetermined future time point, or a determination/detection of EVD and a prognosis of RCC at the predetermined future time point.

“Disease burden” and variations thereof refer to the extent of RCC in a person's body and correlates with pathologic or clinical stage of the cancer at the time of sample collection. The stages are determined by the size of the tumor, whether or not it is localized to the kidney, involvement of the fatty tissues surrounding the kidney, metastasis to distant organs including the heart, lung or bone, and/or whether or not it has spread to the large veins leading to the heart. The determination of disease burden can include other factors including additional biomedical information as is described in detailed herein.

A disease burden can be determined at any time during the course of the RCC disease. It can be used, for example, when RCC is absent, at the time of initial diagnosis, during the course of treatment to monitor the patient's response to the therapy/surgery, and in monitoring RCC recurrence after apparent cure.

The “disease burden vector” or “DBV” provides a continuous burden score. The vector is a model for classifying one group from another, including groups defined by RCC stage. The DBV can be applied to individual samples to obtain a DBV score which reflects that individual's RCC stage or extent of disease.

“Evaluate”, “evaluating”, “evaluation”, and variations thereof encompass “diagnosing,” “prognosing”, predicting disease burden and monitoring of recurrence in a treated individual. “Evaluating” RCC can include any of the following: 1) diagnosing RCC, i.e., initially detecting the presence or absence of RCC, determining a specific stage, type or sub-type, or other classification or characteristic of RCC, and/or determining whether a renal mass, tissue or other biological sample of an individual is benign or malignant; 2) prognosing at time point 1 (TP1), the future outcome of RCC at time point 2 (TP2), i.e., where TP2 may follow RCC therapy such as surgery or resection, and can include follow up of any range of dates (e.g., 1-5, 2-5, 3-5, 4-5, 1-4, 2-4, 3-4, 1-3, 2-3, and 1-2 years) up to 5 years after therapy or surgery; 3) predicting extent of disease or RCC disease burden at the time of sample collection and/or 4) detecting or monitoring RCC progression, remission, or recurrence after apparent cure of RCC, i.e., wherein “monitoring after apparent cure of RCC” means testing an individual a time point after s/he has received successful surgery and/or other treatment for RCC, and when s/he has manifested complete or partial remission, relative to a time point prior to treatment, as reflected by clinical symptoms or other indicators.

As used herein, “additional biomedical information” refers to one or more evaluations of an individual, other than using any of the biomarkers described herein, that are associated with RCC risk. “Additional biomedical information” includes any of the following: physical descriptors of an individual; physical descriptors of a abdominal or renal mass observed by MRI, abdominal ultrasound, or other radiologic imaging; pathologic data from excised tissue, the height and/or weight of an individual; change in weight; the ethnicity of an individual; occupational history; family history of RCC (or other cancer); the presence of a genetic marker(s) correlating with a higher risk of RCC in the individual or a family member; the presence of a abdominal or renal mass; size of mass; location of mass; morphology of mass and associated abdominal region (e.g., as observed through radiologic imaging); clinical symptoms such as hematuria, flank pain, palpable abdominal mass, scrotal varicoeles, lower extremity edema, ascites, hepatic dysfunction, pulmonary emboli, anemia, fever, hypercalcemia, cachexia, erythrocytosis, amyloidosis, thrombocytosis, Polymyalgia rheumatica abdominal pain; and the like. Additional biomedical information can be obtained from an individual using routine techniques known in the art, such as from the individual themselves by use of a routine patient questionnaire or health history questionnaire, etc., or from a medical practitioner, etc. Alternately, additional biomedical information can be obtained from routine imaging techniques, including abdominal ultrasound, MRI, CT imaging, and PET-CT. Testing of biomarker levels in combination with an evaluation of any additional biomedical information, including other laboratory tests, may, for example, improve sensitivity, specificity, and/or AUC for detecting RCC (or other RCC-related uses) as compared to biomarker testing alone or evaluating any particular item of additional biomedical information alone (e.g., ultrasound imaging alone).

The term “area under the curve” or “AUC” refers to the area under the curve of a receiver operating characteristic (ROC) curve, both of which are well known in the art. AUC measures are useful for comparing the accuracy of a classifier across the complete data range. Classifiers with a greater AUC have a greater capacity to classify unknowns correctly between two groups of interest (e.g., RCC samples and normal or control samples). ROC curves are useful for plotting the performance of a particular feature (e.g., any of the biomarkers described herein and/or any item of additional biomedical information) in distinguishing between two populations (e.g., cases having RCC and controls without RCC). Typically, the feature data across the entire population (e.g., the cases and controls) are sorted in ascending order based on the value of a single feature. Then, for each value for that feature, the true positive and false positive rates for the data are calculated. The true positive rate is determined by counting the number of cases above the value for that feature and then dividing by the total number of cases. The false positive rate is determined by counting the number of controls above the value for that feature and then dividing by the total number of controls. Although this definition refers to scenarios in which a feature is elevated in cases compared to controls, this definition also applies to scenarios in which a feature is lower in cases compared to the controls (in such a scenario, samples below the value for that feature would be counted). ROC curves can be generated for a single feature as well as for other single outputs, for example, a combination of two or more features can be mathematically combined (e.g., added, subtracted, multiplied, etc.) to provide a single sum value, and this single sum value can be plotted in a ROC curve. Additionally, any combination of multiple features, in which the combination derives a single output value, can be plotted in a ROC curve. These combinations of features may comprise a test. The ROC curve is the plot of the true positive rate (sensitivity) of a test against the false positive rate (1-specificity) of the test.

As used herein, “detecting” or “determining” with respect to a biomarker value includes the use of both the instrument required to observe and record a signal corresponding to a biomarker value and the material/s required to generate that signal. In various embodiments, the biomarker value is detected using any suitable method, including fluorescence, chemiluminescence, surface plasmon resonance, surface acoustic waves, mass spectrometry, infrared spectroscopy, Raman spectroscopy, atomic force microscopy, scanning tunneling microscopy, electrochemical detection methods, nuclear magnetic resonance, quantum dots, and the like. “Detecting” and “determining,” used interchangeably herein, both refer to the identification or observation of the presence of a biomarker in a biological sample, and/or to the measurement of the biomarker value.

“Solid support” refers herein to any substrate having a surface to which molecules may be attached, directly or indirectly, through either covalent or non-covalent bonds. A “solid support” can have a variety of physical formats, which can include, for example, a membrane; a chip (e.g., a protein chip); a slide (e.g., a glass slide or coverslip); a column; a hollow, solid, semi-solid, pore- or cavity-containing particle, such as, for example, a bead; a gel; a fiber, including a fiber optic material; a matrix; and a sample receptacle. Exemplary sample receptacles include sample wells, tubes, capillaries, vials, and any other vessel, groove or indentation capable of holding a sample. A sample receptacle can be contained on a multi-sample platform, such as a microtiter plate, slide, microfluidics device, and the like. A support can be composed of a natural or synthetic material, an organic or inorganic material. The composition of the solid support on which capture reagents are attached generally depends on the method of attachment (e.g., covalent attachment). Other exemplary receptacles include microdroplets and microfluidic controlled or bulk oil/aqueous emulsions within which assays and related manipulations can occur. Suitable solid supports include, for example, plastics, resins, polysaccharides, silica or silica-based materials, functionalized glass, modified silicon, carbon, metals, inorganic glasses, membranes, nylon, natural fibers (such as, for example, silk, wool and cotton), polymers, and the like. The material composing the solid support can include reactive groups such as, for example, carboxy, amino, or hydroxyl groups, which are used for attachment of the capture reagents. Polymeric solid supports can include, e.g., polystyrene, polyethylene glycol tetraphthalate, polyvinyl acetate, polyvinyl chloride, polyvinyl pyrrolidone, polyacrylonitrile, polymethyl methacrylate, polytetrafluoroethylene, butyl rubber, styrenebutadiene rubber, natural rubber, polyethylene, polypropylene, (poly)tetrafluoroethylene, (poly)vinylidenefluoride, polycarbonate, and polymethylpentene. Suitable solid support particles that can be used include, e.g., encoded particles, such as Luminex®-type encoded particles, magnetic particles, and glass particles.

### Exemplary Uses of Biomarkers

In various exemplary embodiments, methods are provided for diagnosing RCC in an individual by detecting one or more biomarker values corresponding to one or more biomarkers that are present in the circulation of an individual, such as in serum or plasma, by any number of analytical methods, including any of the analytical methods described herein. These biomarkers are, for example, differentially expressed in individuals with RCC as compared to individuals without RCC. Detection of the differential expression of a biomarker in an individual can be used, for example, to permit the early diagnosis of RCC, to prognose future outcome of RCC in an individual following therapy or surgery, determine disease burden, and/or to monitor RCC recurrence after therapy, or for other clinical indications.

Any of the biomarkers described herein may be used in a variety of clinical indications for RCC, including any of the following: detection of RCC (such as in a high-risk or symptomatic individual or population); characterizing RCC (e.g., determining RCC type, sub-type, or stage), such as by determining whether a renal mass is benign or malignant; determining RCC prognosis; determining disease burden, monitoring RCC progression or remission; monitoring for RCC recurrence; monitoring metastasis; treatment selection (e.g., pre- or post-operative chemotherapy selection); monitoring response to a therapeutic agent or other treatment; combining biomarker testing with additional biomedical information, such as the presence of a genetic marker(s) indicating a higher risk for RCC, etc., or with mass size, morphology etc. (such as to provide an assay with increased diagnostic performance); facilitating the diagnosis of a renal mass as malignant or benign; facilitating clinical decision making once a renal mass is observed through imaging; and facilitating decisions regarding clinical follow-up (e.g., whether to refer an individual for surgical resection or systemic treatment). Furthermore, the described biomarkers may also be useful in permitting certain of these uses before indications of RCC are detected by imaging modalities or other clinical correlates, or before symptoms appear.

As an example of the manner in which any of the biomarkers described herein can be used to diagnose RCC, differential expression of one or more of the described biomarkers in an individual who is not known to have RCC may indicate that the individual has RCC, thereby enabling detection of RCC at an early stage of the disease when treatment is most effective, perhaps before the RCC is detected by other means or before symptoms appear. Increased differential expression from “normal” (since some biomarkers may be down-regulated with disease) of one or more of the biomarkers during the course of RCC may be indicative of RCC progression, e.g., metastasis (and thus indicate a poor prognosis), whereas a decrease in the degree to which one or more of the biomarkers is differentially expressed (i.e., in subsequent biomarker tests, the expression level in the individual is moving toward or approaching a “normal” expression level) may be indicative of RCC remission, e.g., surgical cure (and thus indicate a good or better prognosis). Similarly, an increase in the degree to which one or more of the biomarkers is differentially expressed (i.e., in subsequent biomarker tests, the expression level in the individual is moving further away from a “normal” expression level) during the course of RCC treatment may indicate that the RCC is progressing and therefore indicate that the treatment is ineffective, whereas a decrease in differential expression of one or more of the biomarkers during the course of RCC treatment may be indicative of RCC remission and therefore indicate that the treatment is working successfully. Additionally, an increase or decrease in the differential expression of one or more of the biomarkers after an individual has apparently been cured of RCC may be indicative of RCC recurrence. In a situation such as this, for example, the individual can be re-started on therapy (or the therapeutic regimen modified such as to increase dosage amount and/or frequency, if the individual has maintained therapy) or surgical resection at an earlier stage than if the recurrence of RCC was not detected until later. Furthermore, a differential expression level of one or more of the biomarkers in an individual may be predictive of the individual's response to a particular therapeutic agent. In monitoring for RCC recurrence or progression, changes in the biomarker expression levels may indicate the need for repetitive biomarker assays or repeat imaging, such as to determine RCC activity or to determine the need for changes in treatment. Measuring biomarker changes longitudinally within an individual establishes a personal baseline and provides a sensitive method to detect changes that may be evident prior to clinical emergence of altered disease state.

Detection of any of the biomarkers described herein may be particularly useful following, or in conjunction with, RCC treatment, such as to evaluate the success of the treatment or to monitor RCC remission, recurrence, disease burden and/or progression (including metastasis) following treatment. RCC treatment may include, for example, administration of a therapeutic agent to the individual, performance of surgery (e.g., surgical resection of at least a portion of a renal mass), administration of radiation therapy, or any other type of RCC treatment used in the art, and any combination of these treatments. For example, any of the biomarkers may be detected at least once after treatment or may be detected multiple times after treatment (such as at periodic intervals), or may be detected both before and after treatment. Differential expression levels of any of the biomarkers in an individual over time may be indicative of RCC progression, remission, or recurrence, examples of which include any of the following: an increase or decrease in the expression level of the biomarkers after treatment compared with the expression level of the biomarker before treatment; an increase or decrease in the expression level of the biomarker at a later time point after treatment compared with the expression level of the biomarker at an earlier time point after treatment; and a differential expression level of the biomarker at a single time point after treatment compared with normal levels of the biomarker.

As a specific example, the biomarker levels for any of the biomarkers described herein can be determined in pre-surgery and post-surgery serum or plasma samples. An increase in the biomarker expression level(s) in the post-surgery sample compared with the pre-surgery sample can indicate residual RCC or progression of RCC (e.g., unsuccessful surgery), whereas a decrease in the biomarker expression level(s) in the post-surgery sample compared with the pre-surgery sample can indicate regression of RCC and reduction in disease burden (e.g., the surgery successfully removed the RCC mass). Similar analyses of the biomarker levels can be carried out before and after other forms of treatment, such as before and after radiation therapy or administration of a therapeutic agent or cancer vaccine.

In addition to testing biomarker levels as a stand-alone diagnostic test, biomarker levels can also be done in conjunction with determination of SNPs or other genetic lesions or variability that are indicative of increased risk of susceptibility of disease. (See, e.g., Hagenkord, J. et al., Diagnostic Pathology 3:44 (2009)).

The determination of disease burden refers to the determination of extent of RCC or the RCC stage in an individual. It is similar to the determination of RCC stage using the method of diagnosis described herein. It can be done at any time during the course of the disease and/or the recovery therefrom. For example, it can be used at the time of initial diagnosis, during the monitoring of patient treatment with therapy or following surgery, and/or in monitoring RCC recurrence after apparent cure.

The extent of disease is reflected by the size of the tumor, whether or not it is localized to the kidney, involvement of the fatty tissues surrounding the kidney, metastasis to distant organs including the heart, lung or bone, and/or whether or not it has spread to the large veins leading to the heart. The determination of disease burden can include other factors including additional biomedical information as is described in detailed herein.

The disease burden vector or DBV is used to determine, at least in part, the disease burden of a patient. The DBV is a model for classifying different RCC stages from one another. The DBV can be applied to patient samples to obtain the DBV score which reflects that individual's RCC stage, extent of disease or disease burden.

Thus, the method of determining a RCC disease burden in an individual includes the steps of: selecting a RCC disease burden vector (DBV) modeled on biomarkers that correlate with RCC stage; providing an individual's sample suspected of containing said biomarkers; applying the DBV to the sample biomarkers to determine the individual's disease burden vector score (DBV score); and determining the disease burden on the basis of the DBV score. As mentioned above, the determination of the disease burden can further include additional biomedical information.

Detection of any of the biomarkers described herein may be useful after a renal mass has been observed through imaging to aid in the diagnosis of RCC and guide appropriate clinical care of the individual, including care by an appropriate surgical specialist or oncologist.

In addition to testing biomarker levels in conjunction with relevant symptoms or imaging data, information regarding the biomarkers can also be evaluated in conjunction with other types of data, particularly data that indicates an individual's risk for RCC (e.g., patient clinical history, symptoms, family history of RCC, risk factors such as the presence of a genetic marker(s), and/or status of other biomarkers, clinical symptoms, etc.). These various data can be assessed by automated methods, such as a computer program/software, which can be embodied in a computer or other apparatus/device.

Any of the described biomarkers may also be used in imaging tests. For example, an imaging agent can be coupled to any of the described biomarkers, which can be used to aid in RCC diagnosis, to prognose outcome following treatment, to monitor disease burden/progression/remission or metastasis, to monitor for disease recurrence, or to monitor response to therapy, among other uses.

### Detection and Determination of Biomarkers and Biomarker Values

A biomarker value for the biomarkers described herein can be detected using any of a variety of known analytical methods. In one embodiment, a biomarker value is detected using a capture reagent. As used herein, a “capture agent” or “capture reagent” refers to a molecule that is capable of binding specifically to a biomarker. In various embodiments, the capture reagent can be exposed to the biomarker in solution or can be exposed to the biomarker while the capture reagent is immobilized on a solid support. In other embodiments, the capture reagent contains a feature that is reactive with a secondary feature on a solid support. In these embodiments, the capture reagent can be exposed to the biomarker in solution, and then the feature on the capture reagent can be used in conjunction with the secondary feature on the solid support to immobilize the biomarker on the solid support. The capture reagent is selected based on the type of analysis to be conducted. Capture reagents include but are not limited to aptamers, antibodies, antigens, adnectins, ankyrins, other antibody mimetics and other protein scaffolds, autoantibodies, chimeras, small molecules, an F(ab′)₂ fragment, a single chain antibody fragment, an Fv fragment, a single chain Fv fragment, a nucleic acid, a lectin, a ligand-binding receptor, affybodies, nanobodies, imprinted polymers, avimers, peptidomimetics, a hormone receptor, a cytokine receptor, and synthetic receptors, and modifications and fragments of these.

In some embodiments, a biomarker value is detected using a biomarker/capture reagent complex.

In other embodiments, the biomarker value is derived from the biomarker/capture reagent complex and is detected indirectly, such as, for example, as a result of a reaction that is subsequent to the biomarker/capture reagent interaction, but is dependent on the formation of the biomarker/capture reagent complex.

In some embodiments, the biomarker value is detected directly from the biomarker in a biological sample.

In one embodiment, the biomarkers are detected using a multiplexed format that allows for the simultaneous detection of two or more biomarkers in a biological sample. In one embodiment of the multiplexed format, capture reagents are immobilized, directly or indirectly, covalently or non-covalently, in discrete locations on a solid support. In another embodiment, a multiplexed format uses discrete solid supports where each solid support has a unique capture reagent associated with that solid support, such as, for example quantum dots. In another embodiment, an individual device is used for the detection of each one of multiple biomarkers to be detected in a biological sample. Individual devices can be configured to permit each biomarker in the biological sample to be processed simultaneously. For example, a microtiter plate can be used such that each well in the plate is used to uniquely analyze one of multiple biomarkers to be detected in a biological sample.

In one or more of the foregoing embodiments, a fluorescent tag can be used to label a component of the biomarker/capture complex to enable the detection of the biomarker value. In various embodiments, the fluorescent label can be conjugated to a capture reagent specific to any of the biomarkers described herein using known techniques, and the fluorescent label can then be used to detect the corresponding biomarker value. Suitable fluorescent labels include rare earth chelates, fluorescein and its derivatives, rhodamine and its derivatives, dansyl, allophycocyanin, PBXL-3, Qdot 605, Lissamine, phycoerythrin, Texas Red, and other such compounds.

In one embodiment, the fluorescent label is a fluorescent dye molecule. In some embodiments, the fluorescent dye molecule includes at least one substituted indolium ring system in which the substituent on the 3-carbon of the indolium ring contains a chemically reactive group or a conjugated substance. In some embodiments, the dye molecule includes an AlexaFluor molecule, such as, for example, AlexaFluor 488, AlexaFluor 532, AlexaFluor 647, AlexaFluor 680, or AlexaFluor 700. In other embodiments, the dye molecule includes a first type and a second type of dye molecule, such as, e.g., two different AlexaFluor molecules. In other embodiments, the dye molecule includes a first type and a second type of dye molecule, and the two dye molecules have different emission spectra.

Fluorescence can be measured with a variety of instrumentation compatible with a wide range of assay formats. For example, spectrofluorimeters have been designed to analyze microtiter plates, microscope slides, printed arrays, cuvettes, etc. See Principles of Fluorescence Spectroscopy, by J. R. Lakowicz, Springer Science+Business Media, Inc., 2004. See Bioluminescence & Chemiluminescence: Progress & Current Applications; Philip E. Stanley and Larry J. Kricka editors, World Scientific Publishing Company, January 2002.

In one or more of the foregoing embodiments, a chemiluminescence tag can optionally be used to label a component of the biomarker/capture complex to enable the detection of a biomarker value. Suitable chemiluminescent materials include any of oxalyl chloride, Rodamin 6G, Ru(bipy)₃²⁺, TMAE (tetrakis(dimethylamino)ethylene), Pyrogallol (1,2,3-trihydroxibenzene), Lucigenin, peroxyoxalates, Aryl oxalates, Acridinium esters, dioxetanes, and others.

In yet other embodiments, the detection method includes an enzyme/substrate combination that generates a detectable signal that corresponds to the biomarker value. Generally, the enzyme catalyzes a chemical alteration of the chromogenic substrate which can be measured using various techniques, including spectrophotometry, fluorescence, and chemiluminescence. Suitable enzymes include, for example, luciferases, luciferin, malate dehydrogenase, urease, horseradish peroxidase (HRPO), alkaline phosphatase, beta-galactosidase, glucoamylase, lysozyme, glucose oxidase, galactose oxidase, and glucose-6-phosphate dehydrogenase, uricase, xanthine oxidase, lactoperoxidase, microperoxidase, and the like.

In yet other embodiments, the detection method can be a combination of fluorescence, chemiluminescence, radionuclide or enzyme/substrate combinations that generate a measurable signal. Multimodal signaling could have unique and advantageous characteristics in biomarker assay formats.

More specifically, the biomarker values for the biomarkers described herein can be detected using known analytical methods including, singleplex aptamer assays, multiplexed aptamer assays, singleplex or multiplexed immunoassays, mRNA expression profiling, miRNA expression profiling, mass spectrometric analysis, histological/cytological methods, etc. as detailed below.

### Determination of Biomarker Values Using Aptamer-Based Assays

Assays directed to the detection and quantification of physiologically significant molecules in biological samples and other samples are important tools in scientific research and in the health care field. One class of such assays involves the use of a microarray that includes one or more aptamers immobilized on a solid support. The aptamers are each capable of binding to a target molecule in a highly specific manner and with very high affinity. See, e.g., U.S. Pat. No. 5,475,096 entitled “Nucleic Acid Ligands”; see also, e.g., U.S. Pat. No. 6,242,246, U.S. Pat. No. 6,458,543, and U.S. Pat. No. 6,503,715, each of which is entitled “Nucleic Acid Ligand Diagnostic Biochip”. Once the microarray is contacted with a sample, the aptamers bind to their respective target molecules present in the sample and thereby enable a determination of a biomarker value corresponding to a biomarker.

As used herein, an “aptamer” refers to a nucleic acid that has a specific binding affinity for a target molecule. It is recognized that affinity interactions are a matter of degree; however, in this context, the “specific binding affinity” of an aptamer for its target means that the aptamer binds to its target generally with a much higher degree of affinity than it binds to other components in a test sample. An “aptamer” is a set of copies of one type or species of nucleic acid molecule that has a particular nucleotide sequence. An aptamer can include any suitable number of nucleotides, including any number of chemically modified nucleotides. “Aptamers” refers to more than one such set of molecules. Different aptamers can have either the same or different numbers of nucleotides. Aptamers can be DNA or RNA or chemically modified nucleic acids and can be single stranded, double stranded, or contain double stranded regions, and can include higher ordered structures. An aptamer can also be a photoaptamer, where a photoreactive or chemically reactive functional group is included in the aptamer to allow it to be covalently linked to its corresponding target. Any of the aptamer methods disclosed herein can include the use of two or more aptamers that specifically bind the same target molecule. As further described below, an aptamer may include a tag. If an aptamer includes a tag, all copies of the aptamer need not have the same tag. Moreover, if different aptamers each include a tag, these different aptamers can have either the same tag or a different tag.

An aptamer can be identified using any known method, including the SELEX process. Once identified, an aptamer can be prepared or synthesized in accordance with any known method, including chemical synthetic methods and enzymatic synthetic methods.

The terms “SELEX” and “SELEX process” are used interchangeably herein to refer generally to a combination of (1) the selection of aptamers that interact with a target molecule in a desirable manner, for example binding with high affinity to a protein, with (2) the amplification of those selected nucleic acids. The SELEX process can be used to identify aptamers with high affinity to a specific target or biomarker.

SELEX generally includes preparing a candidate mixture of nucleic acids, binding of the candidate mixture to the desired target molecule to form an affinity complex, separating the affinity complexes from the unbound candidate nucleic acids, separating and isolating the nucleic acid from the affinity complex, purifying the nucleic acid, and identifying a specific aptamer sequence. The process may include multiple rounds to further refine the affinity of the selected aptamer. The process can include amplification steps at one or more points in the process. See, e.g., U.S. Pat. No. 5,475,096, entitled “Nucleic Acid Ligands”. The SELEX process can be used to generate an aptamer that covalently binds its target as well as an aptamer that non-covalently binds its target. See, e.g., U.S. Pat. No. 5,705,337 entitled “Systematic Evolution of Nucleic Acid Ligands by Exponential Enrichment: Chemi-SELEX.”

The SELEX process can be used to identify high-affinity aptamers containing modified nucleotides that confer improved characteristics on the aptamer, such as, for example, improved in vivo stability or improved delivery characteristics. Examples of such modifications include chemical substitutions at the ribose and/or phosphate and/or base positions. SELEX process-identified aptamers containing modified nucleotides are described in U.S. Pat. No. 5,660,985, entitled “High Affinity Nucleic Acid Ligands Containing Modified Nucleotides”, which describes oligonucleotides containing nucleotide derivatives chemically modified at the 5′- and 2′-positions of pyrimidines. U.S. Pat. No. 5,580,737, see supra, describes highly specific aptamers containing one or more nucleotides modified with 2′-amino (2′-NH2), 2′-fluoro (2′-F), and/or 2′-O-methyl (2′-OMe). See also, U.S. Patent Application Publication 20090098549, entitled “SELEX and PHOTOSELEX”, which describes nucleic acid libraries having expanded physical and chemical properties and their use in SELEX and photoSELEX.

SELEX can also be used to identify aptamers that have desirable off-rate characteristics. See U.S. Patent Application Publication 20090004667, entitled “Method for Generating Aptamers with Improved Off-Rates”, which describes improved SELEX methods for generating aptamers that can bind to target molecules. Methods for producing aptamers and photoaptamers known as SOMAmers® having slower rates of dissociation from their respective target molecules are described. The methods involve contacting the candidate mixture with the target molecule, allowing the formation of nucleic acid-target complexes to occur, and performing a slow off-rate enrichment process wherein nucleic acid-target complexes with fast dissociation rates will dissociate and not reform, while complexes with slow dissociation rates will remain intact. Additionally, the methods include the use of modified nucleotides in the production of candidate nucleic acid mixtures to generate aptamers with improved off-rate performance.

A variation of this assay employs aptamers that include photoreactive functional groups that enable the aptamers to covalently bind or “photocrosslink” their target molecules. See, e.g., U.S. Pat. No. 6,544,776 entitled “Nucleic Acid Ligand Diagnostic Biochip”. These photoreactive aptamers are also referred to as photoaptamers. See, e.g., U.S. Pat. No. 5,763,177, U.S. Pat. No. 6,001,577, and U.S. Pat. No. 6,291,184, each of which is entitled “Systematic Evolution of Nucleic Acid Ligands by Exponential Enrichment: Photoselection of Nucleic Acid Ligands and Solution SELEX”; see also, e.g., U.S. Pat. No. 6,458,539, entitled “Photoselection of Nucleic Acid Ligands”. After the microarray is contacted with the sample and the photoaptamers have had an opportunity to bind to their target molecules, the photoaptamers are photoactivated, and the solid support is washed to remove any non-specifically bound molecules. Harsh wash conditions may be used, since target molecules that are bound to the photoaptamers are generally not removed, due to the covalent bonds created by the photoactivated functional group(s) on the photoaptamers. In this manner, the assay enables the detection of a biomarker value corresponding to a biomarker in the test sample.

In both of these assay formats, the aptamers are immobilized on the solid support prior to being contacted with the sample. Under certain circumstances, however, immobilization of the aptamers prior to contact with the sample may not provide an optimal assay. For example, pre-immobilization of the aptamers may result in inefficient mixing of the aptamers with the target molecules on the surface of the solid support, perhaps leading to lengthy reaction times and, therefore, extended incubation periods to permit efficient binding of the aptamers to their target molecules. Further, when photoaptamers are employed in the assay and depending upon the material utilized as a solid support, the solid support may tend to scatter or absorb the light used to effect the formation of covalent bonds between the photoaptamers and their target molecules. Moreover, depending upon the method employed, detection of target molecules bound to their aptamers can be subject to imprecision, since the surface of the solid support may also be exposed to and affected by any labeling agents that are used. Finally, immobilization of the aptamers on the solid support generally involves an aptamer-preparation step (i.e., the immobilization) prior to exposure of the aptamers to the sample, and this preparation step may affect the activity or functionality of the aptamers.

Aptamer assays that permit an aptamer to capture its target in solution and then employ separation steps that are designed to remove specific components of the aptamer-target mixture prior to detection have also been described (see U.S. Patent Application Publication 20090042206, entitled “Multiplexed Analyses of Test Samples”). The described aptamer assay methods enable the detection and quantification of a non-nucleic acid target (e.g., a protein target) in a test sample by detecting and quantifying a nucleic acid (i.e., an aptamer). The described methods create a nucleic acid surrogate (i.e., the aptamer) for detecting and quantifying a non-nucleic acid target, thus allowing the wide variety of nucleic acid technologies, including amplification, to be applied to a broader range of desired targets, including protein targets.

Aptamers can be constructed to facilitate the separation of the assay components from an aptamer biomarker complex (or photoaptamer biomarker covalent complex) and permit isolation of the aptamer for detection and/or quantification. In one embodiment, these constructs can include a cleavable or releasable element within the aptamer sequence. In other embodiments, additional functionality can be introduced into the aptamer, for example, a labeled or detectable component, a spacer component, or a specific binding tag or immobilization element. For example, the aptamer can include a tag connected to the aptamer via a cleavable moiety, a label, a spacer component separating the label, and the cleavable moiety. In one embodiment, a cleavable element is a photocleavable linker. The photocleavable linker can be attached to a biotin moiety and a spacer section, can include an NHS group for derivatization of amines, and can be used to introduce a biotin group to an aptamer, thereby allowing for the release of the aptamer later in an assay method.

Homogenous assays, done with all assay components in solution, do not require separation of sample and reagents prior to the detection of signal. These methods are rapid and easy to use. These methods generate signal based on a molecular capture or binding reagent that reacts with its specific target. For RCC, the molecular capture reagents would be an aptamer or an antibody or the like and the specific target would be a RCC biomarker of Table 1.

In one embodiment, a method for signal generation takes advantage of anisotropy signal change due to the interaction of a fluorophore-labeled capture reagent with its specific biomarker target. When the labeled capture reacts with its target, the increased molecular weight causes the rotational motion of the fluorophore attached to the complex to become much slower changing the anisotropy value. By monitoring the anisotropy change, binding events may be used to quantitatively measure the biomarkers in solutions. Other methods include fluorescence polarization assays, molecular beacon methods, time resolved fluorescence quenching, chemiluminescence, fluorescence resonance energy transfer, and the like.

An exemplary solution-based aptamer assay that can be used to detect a biomarker value corresponding to a biomarker in a biological sample includes the following: (a) preparing a mixture by contacting the biological sample with an aptamer that includes a first tag and has a specific affinity for the biomarker, wherein an aptamer affinity complex is formed when the biomarker is present in the sample; (b) exposing the mixture to a first solid support including a first capture element, and allowing the first tag to associate with the first capture element; (c) removing any components of the mixture not associated with the first solid support; (d) attaching a second tag to the biomarker component of the aptamer affinity complex; (e) releasing the aptamer affinity complex from the first solid support; (f) exposing the released aptamer affinity complex to a second solid support that includes a second capture element and allowing the second tag to associate with the second capture element; (g) removing any non-complexed aptamer from the mixture by partitioning the non-complexed aptamer from the aptamer affinity complex; (h) eluting the aptamer from the solid support; and (i) detecting the biomarker by detecting the aptamer component of the aptamer affinity complex.

Any means known in the art can be used to detect a biomarker value by detecting the aptamer component of an aptamer affinity complex. A number of different detection methods can be used to detect the aptamer component of an affinity complex, such as, for example, hybridization assays, mass spectroscopy, or QPCR. In some embodiments, nucleic acid sequencing methods can be used to detect the aptamer component of an aptamer affinity complex and thereby detect a biomarker value. Briefly, a test sample can be subjected to any kind of nucleic acid sequencing method to identify and quantify the sequence or sequences of one or more aptamers present in the test sample.

In some embodiments, the sequence includes the entire aptamer molecule or any portion of the molecule that may be used to uniquely identify the molecule. In other embodiments, the identifying sequencing is a specific sequence added to the aptamer; such sequences are often referred to as “tags,” “barcodes,” or “zipcodes.”

In some embodiments, the sequencing method includes enzymatic steps to amplify the aptamer sequence or to convert any kind of nucleic acid, including RNA and DNA that contain chemical modifications to any position, to any other kind of nucleic acid appropriate for sequencing.

In some embodiments, the sequencing method includes one or more cloning steps. In other embodiments the sequencing method includes a direct sequencing method without cloning.

In some embodiments, the sequencing method includes a directed approach with specific primers that target one or more aptamers in the test sample. In other embodiments, the sequencing method includes a shotgun approach that targets all aptamers in the test sample.

In some embodiments, the sequencing method includes enzymatic steps to amplify the molecule targeted for sequencing. In other embodiments, the sequencing method directly sequences single molecules.

An exemplary nucleic acid sequencing-based method that can be used to detect a biomarker value corresponding to a biomarker in a biological sample includes the following: (a) converting a mixture of aptamers that contain chemically modified nucleotides to unmodified nucleic acids with an enzymatic step; (b) shotgun sequencing the resulting unmodified nucleic acids with a massively parallel sequencing platform such as, for example, the 454 Sequencing System (454 Life Sciences/Roche), the Illumina Sequencing System (Illumina), the ABI SOLiD SequencingSystem (Applied Biosystems), the HeliScope Single Molecule Sequencer (Helicos Biosciences), or the Pacific Biosciences Real Time Single-Molecule Sequencing System (Pacific BioSciences) or the Polonator G Sequencing System (Dover Systems); and (c) identifying and quantifying the aptamers present in the mixture by specific sequence and sequence count.

### Determination of Biomarker Values Using Immunoassays

Immunoassay methods are based on the reaction of an antibody to its corresponding target or analyte and can detect the analyte in a sample depending on the specific assay format. To improve specificity and sensitivity of an assay method based on immuno-reactivity, monoclonal antibodies are often used because of their specific epitope recognition. Polyclonal antibodies have also been successfully used in various immunoassays because of their increased affinity for the target as compared to monoclonal antibodies. Immunoassays have been designed for use with a wide range of biological sample matrices. Immunoassay formats have been designed to provide qualitative, semi-quantitative, and quantitative results.

Quantitative results are generated through the use of a standard curve created with known concentrations of the specific analyte to be detected. The response or signal from an unknown sample is plotted onto the standard curve, and a quantity or value corresponding to the target in the unknown sample is established.

Numerous immunoassay formats have been designed. ELISA or EIA can be quantitative for the detection of an analyte. This method relies on attachment of a label to either the analyte or the antibody and the label component includes, either directly or indirectly, an enzyme. ELISA tests may be formatted for direct, indirect, competitive, or sandwich detection of the analyte. Other methods rely on labels such as, for example, radioisotopes (I¹²⁵) or fluorescence. Additional techniques include, for example, agglutination, nephelometry, turbidimetry, Western blot, immunoprecipitation, immunocytochemistry, immunohistochemistry, flow cytometry, Luminex assay, and others (see ImmunoAssay: A Practical Guide, edited by Brian Law, published by Taylor & Francis, Ltd., 2005 edition).

Exemplary assay formats include enzyme-linked immunosorbent assay (ELISA), radioimmunoassay, fluorescent, chemiluminescence, and fluorescence resonance energy transfer (FRET) or time resolved-FRET (TR-FRET) immunoassays. Examples of procedures for detecting biomarkers include biomarker immunoprecipitation followed by quantitative methods that allow size and peptide level discrimination, such as gel electrophoresis, capillary electrophoresis, planar electrochromatography, and the like.

Methods of detecting and/or quantifying a detectable label or signal generating material depend on the nature of the label. The products of reactions catalyzed by appropriate enzymes (where the detectable label is an enzyme; see above) can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers.

Any of the methods for detection can be performed in any format that allows for any suitable preparation, processing, and analysis of the reactions. This can be, for example, in multi-well assay plates (e.g., 96 wells or 384 wells) or using any suitable array or microarray. Stock solutions for various agents can be made manually or robotically, and all subsequent pipetting, diluting, mixing, distribution, washing, incubating, sample readout, data collection and analysis can be done robotically using commercially available analysis software, robotics, and detection instrumentation capable of detecting a detectable label.

### Determination of Biomarker Values Using Gene Expression Profiling

Measuring mRNA in a biological sample may be used as a surrogate for detection of the level of the corresponding protein in the biological sample. Thus, any of the biomarkers or biomarker panels described herein can also be detected by detecting the appropriate RNA.

mRNA expression levels are measured by reverse transcription quantitative polymerase chain reaction (RT-PCR followed with qPCR). RT-PCR is used to create a cDNA from the mRNA. The cDNA may be used in a qPCR assay to produce fluorescence as the DNA amplification process progresses. By comparison to a standard curve, qPCR can produce an absolute measurement such as number of copies of mRNA per cell. Northern blots, microarrays, Invader assays, and RT-PCR combined with capillary electrophoresis have all been used to measure expression levels of mRNA in a sample. See Gene Expression Profiling: Methods and Protocols, Richard A. Shimkets, editor, Humana Press, 2004.

miRNA molecules are small RNAs that are non-coding but may regulate gene expression. Any of the methods suited to the measurement of mRNA expression levels can also be used for the corresponding miRNA. Recently many laboratories have investigated the use of miRNAs as biomarkers for disease. Many diseases involve wide-spread transcriptional regulation, and it is not surprising that miRNAs might find a role as biomarkers. The connection between miRNA concentrations and disease is often even less clear than the connections between protein levels and disease, yet the value of miRNA biomarkers might be substantial. Of course, as with any RNA expressed differentially during disease, the problems facing the development of an in vitro diagnostic product will include the requirement that the miRNAs survive in the diseased cell and are easily extracted for analysis, or that the miRNAs are released into blood or other matrices where they must survive long enough to be measured. Protein biomarkers have similar requirements, although many potential protein biomarkers are secreted intentionally at the site of pathology and function, during disease, in a paracrine fashion. Many potential protein biomarkers are designed to function outside the cells within which those proteins are synthesized.

### Detection of Biomarkers Using In Vivo Molecular Imaging Technologies

Any of the described biomarkers (see Table 1) may also be used in molecular imaging tests. For example, an imaging agent can be coupled to any of the described biomarkers, which can be used to aid in RCC diagnosis, prognosis, to monitor disease burden/progression/remission or metastasis, to monitor for disease recurrence, or to monitor response to therapy, among other uses.

In vivo imaging technologies provide non-invasive methods for determining the state of a particular disease in the body of an individual. For example, entire portions of the body, or even the entire body, may be viewed as a three dimensional image, thereby providing valuable information concerning morphology and structures in the body. Such technologies may be combined with the detection of the biomarkers described herein to provide information concerning the RCC status, in particular the RCC status, of an individual.

The use of in vivo molecular imaging technologies is expanding due to various advances in technology. These advances include the development of new contrast agents or labels, such as radiolabels and/or fluorescent labels, which can provide strong signals within the body; and the development of powerful new imaging technology, which can detect and analyze these signals from outside the body, with sufficient sensitivity and accuracy to provide useful information. The contrast agent can be visualized in an appropriate imaging system, thereby providing an image of the portion or portions of the body in which the contrast agent is located. The contrast agent may be bound to or associated with a capture reagent, such as an aptamer or an antibody, for example, and/or with a peptide or protein, or an oligonucleotide (for example, for the detection of gene expression), or a complex containing any of these with one or more macromolecules and/or other particulate forms.

The contrast agent may also feature a radioactive atom that is useful in imaging. Suitable radioactive atoms include technetium-99m or iodine-123 for scintigraphic studies. Other readily detectable moieties include, for example, spin labels for magnetic resonance imaging (MRI) such as, for example, iodine-123 again, iodine-131, indium-111, fluorine-19, carbon-13, nitrogen-15, oxygen-17, gadolinium, manganese or iron. Such labels are well known in the art and could easily be selected by one of ordinary skill in the art.

Standard imaging techniques include but are not limited to magnetic resonance imaging, contrast-enhanced abdominal or transvaginal ultrasound, computed tomography (CT) scanning, positron emission tomography (PET), single photon emission computed tomography (SPECT), and the like. For diagnostic in vivo imaging, the type of detection instrument available is a major factor in selecting a given contrast agent, such as a given radionuclide and the particular biomarker that it is used to target (protein, mRNA, and the like). The radionuclide chosen typically has a type of decay that is detectable by a given type of instrument. Also, when selecting a radionuclide for in vivo diagnosis, its half-life should be long enough to enable detection at the time of maximum uptake by the target tissue but short enough that deleterious radiation of the host is minimized.

Exemplary imaging techniques include but are not limited to PET and SPECT, which are imaging techniques in which a radionuclide is synthetically or locally administered to an individual. The subsequent uptake of the radiotracer is measured over time and used to obtain information about the targeted tissue and the biomarker. Because of the high-energy (gamma-ray) emissions of the specific isotopes employed and the sensitivity and sophistication of the instruments used to detect them, the two-dimensional distribution of radioactivity may be inferred from outside of the body.

Commonly used positron-emitting nuclides in PET include, for example, carbon-11, nitrogen-13, oxygen-15, and fluorine-18. Isotopes that decay by electron capture and/or gamma-emission are used in SPECT and include, for example iodine-123 and technetium-99m. An exemplary method for labeling amino acids with technetium-99m is the reduction of pertechnetate ion in the presence of a chelating precursor to form the labile technetium-99m-precursor complex, which, in turn, reacts with the metal binding group of a bifunctionally modified chemotactic peptide to form a technetium-99m-chemotactic peptide conjugate.

Antibodies are frequently used for such in vivo imaging diagnostic methods. The preparation and use of antibodies for in vivo diagnosis is well known in the art. Labeled antibodies which specifically bind any of the biomarkers in Table 1 can be injected into an individual suspected of having a certain type of cancer (e.g., RCC), detectable according to the particular biomarker used, for the purpose of diagnosing or evaluating the disease burden or status of the individual. The label used will be selected in accordance with the imaging modality to be used, as previously described. Localization of the label permits determination of the spread of the RCC. The amount of label within an organ or tissue also allows determination of the presence or absence of RCC in that organ or tissue.

Similarly, aptamers may be used for such in vivo imaging diagnostic methods. For example, an aptamer that was used to identify a particular biomarker described in Table 1 (and therefore binds specifically to that particular biomarker) may be appropriately labeled and injected into an individual suspected of having RCC, detectable according to the particular biomarker, for the purpose of diagnosing or evaluating the RCC status of the individual. The label used will be selected in accordance with the imaging modality to be used, as previously described. Localization of the label permits determination of the spread of the RCC. The amount of label within an organ or tissue also allows determination of the presence or absence of RCC in that organ or tissue. Aptamer-directed imaging agents could have unique and advantageous characteristics relating to tissue penetration, tissue distribution, kinetics, elimination, potency, and selectivity as compared to other imaging agents.

Such techniques may also optionally be performed with labeled oligonucleotides, for example, for detection of gene expression through imaging with antisense oligonucleotides. These methods are used for in situ hybridization, for example, with fluorescent molecules or radionuclides as the label. Other methods for detection of gene expression include, for example, detection of the activity of a reporter gene.

Another general type of imaging technology is optical imaging, in which fluorescent signals within the subject are detected by an optical device that is external to the subject. These signals may be due to actual fluorescence and/or to bioluminescence. Improvements in the sensitivity of optical detection devices have increased the usefulness of optical imaging for in vivo diagnostic assays.

The use of in vivo molecular biomarker imaging is increasing, including for clinical trials, for example, to more rapidly measure clinical efficacy in trials for new disease therapies and/or to avoid prolonged treatment with a placebo for those diseases, such as multiple sclerosis, in which such prolonged treatment may be considered to be ethically questionable.

For a review of other techniques, see N. Blow, Nature Methods, 6, 465-469, 2009.

### Determination of Biomarker Values Using Histology or Cytology Methods

For evaluation of RCC, a variety of tissue samples may be used in histological or cytological methods. Sample selection depends on the primary tumor location and sites of metastases. For example, fine needle aspirates, cutting needles, core biopsies and resected tumor tissue can be used for histology. Any of the biomarkers identified herein that were shown to be up-regulated in the individuals with RCC EVD or increased disease burden can be used to stain a histological specimen as an indication of disease.

In one embodiment, one or more capture reagents specific to the corresponding biomarker is used in a cytological evaluation of a renal cell sample and may include one or more of the following: collecting a cell sample, fixing the cell sample, dehydrating, clearing, immobilizing the cell sample on a microscope slide, permeabilizing the cell sample, treating for analyte retrieval, staining, destaining, washing, blocking, and reacting with one or more capture reagent/s in a buffered solution. In another embodiment, the cell sample is produced from a cell block.

In another embodiment, one or more capture reagents specific to the corresponding biomarker is used in a histological evaluation of a renal tissue sample and may include one or more of the following: collecting a tissue specimen, fixing the tissue sample, dehydrating, clearing, immobilizing the tissue sample on a microscope slide, permeabilizing the tissue sample, treating for analyte retrieval, staining, destaining, washing, blocking, rehydrating, and reacting with capture reagent/s in a buffered solution. In another embodiment, fixing and dehydrating are replaced with freezing.

In another embodiment, the one or more aptamers specific to the corresponding biomarker is reacted with the histological or cytological sample and can serve as the nucleic acid target in a nucleic acid amplification method. Suitable nucleic acid amplification methods include, for example, PCR, q-beta replicase, rolling circle amplification, strand displacement, helicase dependent amplification, loop mediated isothermal amplification, ligase chain reaction, and restriction and circularization aided rolling circle amplification.

In one embodiment, the one or more capture reagent/s specific to the corresponding biomarkers for use in the histological or cytological evaluation are mixed in a buffered solution that can include any of the following: blocking materials, competitors, detergents, stabilizers, carrier nucleic acid, polyanionic materials, etc.

A “cytology protocol” generally includes sample collection, sample fixation, sample immobilization, and staining. “Cell preparation” can include several processing steps after sample collection, including the use of one or more slow off-rate aptamers for the staining of the prepared cells.

Sample collection can include directly placing the sample in an untreated transport container, placing the sample in a transport container containing some type of media, or placing the sample directly onto a slide (immobilization) without any treatment or fixation.

Sample immobilization can be improved by applying a portion of the collected specimen to a glass slide that is treated with polylysine, gelatin, or a silane. Slides can be prepared by smearing a thin and even layer of cells across the slide. Care is generally taken to minimize mechanical distortion and drying artifacts. Liquid specimens can be processed in a cell block method. Or, alternatively, liquid specimens can be mixed 1:1 with the fixative solution for about 10 minutes at room temperature.

Cell blocks can be prepared from residual effusions, sputum, urine sediments, gastrointestinal fluids, cell scraping, ascites, or fine needle aspirates. Cells are concentrated or packed by centrifugation or membrane filtration. A number of methods for cell block preparation have been developed. Representative procedures include the fixed sediment, bacterial agar, or membrane filtration methods. In the fixed sediment method, the cell sediment is mixed with a fixative like Bouins, picric acid, or buffered formalin and then the mixture is centrifuged to pellet the fixed cells. The supernatant is removed, drying the cell pellet as completely as possible. The pellet is collected and wrapped in lens paper and then placed in a tissue cassette. The tissue cassette is placed in a jar with additional fixative and processed as a tissue sample. Agar method is very similar but the pellet is removed and dried on paper towel and then cut in half. The cut side is placed in a drop of melted agar on a glass slide and then the pellet is covered with agar making sure that no bubbles form in the agar. The agar is allowed to harden and then any excess agar is trimmed away. This is placed in a tissue cassette and the tissue process completed. Alternatively, the pellet may be directly suspended in 2% liquid agar at 65° C. and the sample centrifuged. The agar cell pellet is allowed to solidify for an hour at 4° C. The solid agar may be removed from the centrifuge tube and sliced in half. The agar is wrapped in filter paper and then the tissue cassette. Processing from this point forward is as described above. Centrifugation can be replaced in any these procedures with membrane filtration. Any of these processes may be used to generate a “cell block sample.”

Cell blocks can be prepared using specialized resin including Lowicryl resins, LR White, LR Gold, Unicryl, and MonoStep. These resins have low viscosity and can be polymerized at low temperatures and with ultra violet (UV) light. The embedding process relies on progressively cooling the sample during dehydration, transferring the sample to the resin, and polymerizing a block at the final low temperature at the appropriate UV wavelength.

Cell block sections can be stained with hematoxylin-eosin for cytomorphological examination while additional sections are used for examination for specific markers.

Whether the process is cytological or histological, the sample may be fixed prior to additional processing to prevent sample degradation. This process is called “fixation” and describes a wide range of materials and procedures that may be used interchangeably. The sample fixation protocol and reagents are best selected empirically based on the targets to be detected and the specific cell/tissue type to be analyzed. Sample fixation relies on reagents such as ethanol, polyethylene glycol, methanol, formalin, or isopropanol. The samples should be fixed as soon after collection and affixation to the slide as possible. However, the fixative selected can introduce structural changes into various molecular targets making their subsequent detection more difficult. The fixation and immobilization processes and their sequence can modify the appearance of the cell and these changes must be anticipated and recognized by the cytotechnologist. Fixatives can cause shrinkage of certain cell types and cause the cytoplasm to appear granular or reticular. Many fixatives function by crosslinking cellular components. This can damage or modify specific epitopes, generate new epitopes, cause molecular associations, and reduce membrane permeability. Formalin fixation is one of the most common cytological and histological approaches. Formalin forms methyl bridges between neighboring proteins or within proteins. Precipitation or coagulation is also used for fixation and ethanol is frequently used in this type of fixation. A combination of crosslinking and precipitation can also be used for fixation. A strong fixation process is best at preserving morphological information while a weaker fixation process is best for the preservation of molecular targets.

A representative fixative is 50% absolute ethanol, 2 mM polyethylene glycol (PEG), 1.85% formaldehyde. Variations on this formulation include ethanol (50% to 95%), methanol (20%-50%), and formalin (formaldehyde) only. Another common fixative is 2% PEG 1500, 50% ethanol, and 3% methanol. Slides are placed in the fixative for about 10 to 15 minutes at room temperature and then removed and allowed to dry. Once slides are fixed they can be rinsed with a buffered solution like PBS.

A wide range of dyes can be used to differentially highlight and contrast or “stain” cellular, sub-cellular, and tissue features or morphological structures. Hematoylin is used to stain nuclei a blue or black color. Orange G-6 and Eosin Azure both stain the cell's cytoplasm. Orange G stains keratin and glycogen containing cells yellow. Eosin Y is used to stain nucleoli, cilia, red blood cells, and superficial epithelial squamous cells. Romanowsky stains are used for air dried slides and are useful in enhancing pleomorphism and distinguishing extracellular from intracytoplasmic material.

The staining process can include a treatment to increase the permeability of the cells to the stain. Treatment of the cells with a detergent can be used to increase permeability. To increase cell and tissue permeability, fixed samples can be further treated with solvents, saponins, or non-ionic detergents. Enzymatic digestion can also improve the accessibility of specific targets in a tissue sample.

After staining, the sample is dehydrated using a succession of alcohol rinses with increasing alcohol concentration. The final wash is done with xylene or a xylene substitute, such as a citrus terpene, that has a refractive index close to that of the coverslip to be applied to the slide. This final step is referred to as clearing. Once the sample is dehydrated and cleared, a mounting medium is applied. The mounting medium is selected to have a refractive index close to the glass and is capable of bonding the coverslip to the slide. It will also inhibit the additional drying, shrinking, or fading of the cell sample.

Regardless of the stains or processing used, the final evaluation of the renal cytological specimen is made by some type of microscopy to permit a visual inspection of the morphology and a determination of the marker's presence or absence. Exemplary microscopic methods include brightfield, phase contrast, fluorescence, and differential interference contrast.

If secondary tests are required on the sample after examination, the coverslip may be removed and the slide destained. Destaining involves using the original solvent systems used in staining the slide originally without the added dye and in a reverse order to the original staining procedure. Destaining may also be completed by soaking the slide in an acid alcohol until the cells are colorless. Once colorless the slides are rinsed well in a water bath and the second staining procedure applied.

In addition, specific molecular differentiation may be possible in conjunction with the cellular morphological analysis through the use of specific molecular reagents such as antibodies or nucleic acid probes or aptamers. This improves the accuracy of diagnostic cytology. Micro-dissection can be used to isolate a subset of cells for additional evaluation, in particular, for genetic evaluation of abnormal chromosomes, gene expression, or mutations.

Preparation of a tissue sample for histological evaluation involves fixation, dehydration, infiltration, embedding, and sectioning. The fixation reagents used in histology are very similar or identical to those used in cytology and have the same issues of preserving morphological features at the expense of molecular ones such as individual proteins. Time can be saved if the tissue sample is not fixed and dehydrated but instead is frozen and then sectioned while frozen. This is a more gentle processing procedure and can preserve more individual markers. However, freezing is not acceptable for long term storage of a tissue sample as subcellular information is lost due to the introduction of ice crystals. Ice in the frozen tissue sample also prevents the sectioning process from producing a very thin slice and thus some microscopic resolution and imaging of subcellular structures can be lost. In addition to formalin fixation, osmium tetroxide is used to fix and stain phospholipids (membranes).

Dehydration of tissues is accomplished with successive washes of increasing alcohol concentration. Clearing employs a material that is miscible with alcohol and the embedding material and involves a stepwise process starting at 50:50 alcohol:clearing reagent and then 100% clearing agent (xylene or xylene substitute). Infiltration involves incubating the tissue with a liquid form of the embedding agent (warm wax, nitrocellulose solution) first at 50:50 embedding agent: clearing agent and the 100% embedding agent. Embedding is completed by placing the tissue in a mold or cassette and filling with melted embedding agent such as wax, agar, or gelatin. The embedding agent is allowed to harden. The hardened tissue sample may then be sliced into thin section for staining and subsequent examination.

Prior to staining, the tissue section is dewaxed and rehydrated. Xylene is used to dewax the section, one or more changes of xylene may be used, and the tissue is rehydrated by successive washes in alcohol of decreasing concentration. Prior to dewax, the tissue section may be heat immobilized to a glass slide at about 80° C. for about 20 minutes.

Laser capture micro-dissection allows the isolation of a subset of cells for further analysis from a tissue section.

As in cytology, to enhance the visualization of the microscopic features, the tissue section or slice can be stained with a variety of stains. A large menu of commercially available stains can be used to enhance or identify specific features.

To further increase the interaction of molecular reagents with cytological or histological samples, a number of techniques for “analyte retrieval” have been developed. The first such technique uses high temperature heating of a fixed sample. This method is also referred to as heat-induced epitope retrieval or HIER. A variety of heating techniques have been used, including steam heating, microwaving, autoclaving, water baths, and pressure cooking or a combination of these methods of heating. Analyte retrieval solutions include, for example, water, citrate, and normal saline buffers. The key to analyte retrieval is the time at high temperature but lower temperatures for longer times have also been successfully used. Another key to analyte retrieval is the pH of the heating solution. Low pH has been found to provide the best immunostaining but also gives rise to backgrounds that frequently require the use of a second tissue section as a negative control. The most consistent benefit (increased immunostaining without increase in background) is generally obtained with a high pH solution regardless of the buffer composition. The analyte retrieval process for a specific target is empirically optimized for the target using heat, time, pH, and buffer composition as variables for process optimization. Using the microwave analyte retrieval method allows for sequential staining of different targets with antibody reagents. But the time required to achieve antibody and enzyme complexes between staining steps has also been shown to degrade cell membrane analytes. Microwave heating methods have improved in situ hybridization methods as well.

To initiate the analyte retrieval process, the section is first dewaxed and hydrated. The slide is then placed in 10 mM sodium citrate buffer pH 6.0 in a dish or jar. A representative procedure uses an 1100 W microwave and microwaves the slide at 100% power for 2 minutes followed by microwaving the slides using 20% power for 18 minutes after checking to be sure the slide remains covered in liquid. The slide is then allowed to cool in the uncovered container and then rinsed with distilled water. HIER may be used in combination with an enzymatic digestion to improve the reactivity of the target to immunochemical reagents.

One such enzymatic digestion protocol uses proteinase K. A 20 μg/ml concentration of proteinase K is prepared in 50 mM Tris Base, 1 mM EDTA, 0.5% Triton X-100, pH 8.0 buffer. The process first involves dewaxing sections in 2 changes of xylene, 5 minutes each. Then the sample is hydrated in 2 changes of 100% ethanol for 3 minutes each, 95% and 80% ethanol for 1 minute each, and then rinsed in distilled water. Sections are covered with Proteinase K working solution and incubated 10-20 minutes at 37° C. in humidified chamber (optimal incubation time may vary depending on tissue type and degree of fixation). The sections are cooled at room temperature for 10 minutes and then rinsed in PBS Tween 20 for 2×2 min. If desired, sections can be blocked to eliminate potential interference from endogenous compounds and enzymes. The section is then incubated with primary antibody at appropriate dilution in primary antibody dilution buffer for 1 hour at room temperature or overnight at 4° C. The section is then rinsed with PBS Tween 20 for 2×2 min. Additional blocking can be performed, if required for the specific application, followed by additional rinsing with PBS Tween 20 for 3×2 min and then finally the immunostaining protocol completed.

A simple treatment with 1% SDS at room temperature has also been demonstrated to improve immunohistochemical staining. Analyte retrieval methods have been applied to slide mounted sections as well as free floating sections. Another treatment option is to place the slide in a jar containing citric acid and 0.1 Nonident P40 at pH 6.0 and heating to 95° C. The slide is then washed with a buffer solution like PBS.

For immunological staining of tissues it may be useful to block non-specific association of the antibody with tissue proteins by soaking the section in a protein solution like serum or non-fat dry milk.

Blocking reactions may include the need to do any of the following, either alone or in combination: reduce the level of endogenous biotin; eliminate endogenous charge effects; inactivate endogenous nucleases; and inactivate endogenous enzymes like peroxidase and alkaline phosphatase. Endogenous nucleases may be inactivated by degradation with proteinase K, by heat treatment, use of a chelating agent such as EDTA or EGTA, the introduction of carrier DNA or RNA, treatment with a chaotrope such as urea, thiourea, guanidine hydrochloride, guanidine thiocyanate, lithium perchlorate, etc, or diethyl pyrocarbonate. Alkaline phosphatase may be inactivated by treatment with 0.1N HCl for 5 minutes at room temperature or treatment with 1 mM levamisole. Peroxidase activity may be eliminated by treatment with 0.03% hydrogen peroxide. Endogenous biotin may be blocked by soaking the slide or section in an avidin (streptavidin, neutravidin may be substituted) solution for at least 15 minutes at room temperature. The slide or section is then washed for at least 10 minutes in buffer. This may be repeated at least three times. Then the slide or section is soaked in a biotin solution for 10 minutes. This may be repeated at least three times with a fresh biotin solution each time. The buffer wash procedure is repeated. Blocking protocols should be minimized to prevent damaging either the cell or tissue structure or the target or targets of interest but one or more of these protocols could be combined to “block” a slide or section prior to reaction with one or more slow off-rate aptamers. See Basic Medical Histology: the Biology of Cells, Tissues and Organs, authored by Richard G. Kessel, Oxford University Press, 1998.

### Determination of Biomarker Values Using Mass Spectrometry Methods

A variety of configurations of mass spectrometers can be used to detect biomarker values. Several types of mass spectrometers are available or can be produced with various configurations. In general, a mass spectrometer has the following major components: a sample inlet, an ion source, a mass analyzer, a detector, a vacuum system, and instrument-control system, and a data system. Differences in the sample inlet, ion source, and mass analyzer generally define the type of instrument and its capabilities. For example, an inlet can be a capillary-column liquid chromatography source or can be a direct probe or stage such as used in matrix-assisted laser desorption. Common ion sources are, for example, electrospray, including nanospray and microspray or matrix-assisted laser desorption. Common mass analyzers include a quadrupole mass filter, ion trap mass analyzer and time-of-flight mass analyzer. Additional mass spectrometry methods are well known in the art (see Burlingame et al. Anal. Chem. 70:647 R-716R (1998); Kinter and Sherman, New York (2000)).

Protein biomarkers and biomarker values can be detected and measured by any of the following: electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), tandem time-of-flight (TOF/TOF) technology, called ultraflex III TOF/TOF, atmospheric pressure chemical ionization mass spectrometry (APCI-MS), APCI-MS/MS, APCI-(MS)^{N}, atmospheric pressure photoionization mass spectrometry (APPI-MS), APPI-MS/MS, and APPI-(MS)^{N}, quadrupole mass spectrometry, Fourier transform mass spectrometry (FTMS), quantitative mass spectrometry, and ion trap mass spectrometry.

Sample preparation strategies are used to label and enrich samples before mass spectroscopic characterization of protein biomarkers and determination biomarker values. Labeling methods include but are not limited to isobaric tag for relative and absolute quantitation (iTRAQ) and stable isotope labeling with amino acids in cell culture (SILAC). Capture reagents used to selectively enrich samples for candidate biomarker proteins prior to mass spectroscopic analysis include but are not limited to aptamers, antibodies, nucleic acid probes, chimeras, small molecules, an F(ab′)₂ fragment, a single chain antibody fragment, an Fv fragment, a single chain Fv fragment, a nucleic acid, a lectin, a ligand-binding receptor, affybodies, nanobodies, ankyrins, domain antibodies, alternative antibody scaffolds (e.g. diabodies etc) imprinted polymers, avimers, peptidomimetics, peptoids, peptide nucleic acids, threose nucleic acid, a hormone receptor, a cytokine receptor, and synthetic receptors, and modifications and fragments of these.

### Determination of Biomarker Values Using a Proximity Ligation Assay

A proximity ligation assay can be used to determine biomarker values. Briefly, a test sample is contacted with a pair of affinity probes that may be a pair of antibodies or a pair of aptamers, with each member of the pair extended with an oligonucleotide. The targets for the pair of affinity probes may be two distinct determinates on one protein or one determinate on each of two different proteins, which may exist as homo- or hetero-multimeric complexes. When probes bind to the target determinates, the free ends of the oligonucleotide extensions are brought into sufficiently close proximity to hybridize together. The hybridization of the oligonucleotide extensions is facilitated by a common connector oligonucleotide which serves to bridge together the oligonucleotide extensions when they are positioned in sufficient proximity. Once the oligonucleotide extensions of the probes are hybridized, the ends of the extensions are joined together by enzymatic DNA ligation.

Each oligonucleotide extension comprises a primer site for PCR amplification. Once the oligonucleotide extensions are ligated together, the oligonucleotides form a continuous DNA sequence which, through PCR amplification, reveals information regarding the identity and amount of the target protein as well as information regarding protein-protein interactions where the target determinates are on two different proteins. Proximity ligation can provide a highly sensitive and specific assay for real-time protein concentration and interaction information through use of real-time PCR. Probes that do not bind the determinates of interest do not have the corresponding oligonucleotide extensions brought into proximity and no ligation or PCR amplification can proceed, resulting in no signal being produced.

The foregoing assays enable the detection of biomarker values that are useful in methods for evaluating or diagnosing RCC, where the methods comprise detecting, in a biological sample from an individual, at least N biomarker values that each correspond to a biomarker selected from the group consisting of the biomarkers provided in Table 1, wherein a classification, as described in detail below, using the biomarker values indicates whether the individual has RCC EVD. While certain of the described RCC biomarkers are useful alone for detecting, evaluating and diagnosing RCC, methods are also described herein for the grouping of multiple subsets of the RCC biomarkers that are each useful as a panel of three or more biomarkers. In accordance with any of the methods described herein, biomarker values can be detected and classified individually or they can be detected and classified collectively, as for example in a multiplex assay format.

In another aspect, methods are provided for detecting an absence of RCC, the methods comprising detecting, in a biological sample from an individual, at least N biomarker values that each correspond to a biomarker selected from the group consisting of the biomarkers provided in Table 1, wherein a classification, as described in detail below, of the biomarker values indicates an absence of RCC in the individual. In accordance with any of the methods described herein, biomarker values can be detected and classified individually or they can be detected and classified collectively, as for example in a multiplex assay format.

### Classification of Biomarkers and Calculation of RCC Disease Scores

A biomarker “signature” for a given evaluation test contains a set of markers, each marker having different levels in the populations of interest. Different levels, in this context, may refer to different means of the marker levels for the individuals in two or more groups, or different variances in the two or more groups, or a combination of both. For the simplest form of an evaluation test, these markers can be used to assign an unknown sample from an individual into one of two groups, either diseased or not diseased. The assignment of a sample into one of two or more groups is known as classification, and the procedure used to accomplish this assignment is known as a classifier or a classification method. Classification methods may also be referred to as scoring methods. There are many classification methods that can be used to construct an evaluation classifier from a set of biomarker values. In general, classification methods are most easily performed using supervised learning techniques where a data set is collected using samples obtained from individuals within two (or more, for multiple classification states) distinct groups one wishes to distinguish. Since the class (group or population) to which each sample belongs is known in advance for each sample, the classification method can be trained to give the desired classification response. It is also possible to use unsupervised learning techniques to produce a disease classifier, such as a prognostic classifier.

Common approaches for developing evaluation classifiers include decision trees; bagging+boosting+forests; rule inference based learning; Parzen Windows; linear models; logistic; neural network methods; unsupervised clustering; K-means; hierarchical ascending/descending; semi-supervised learning; prototype methods; nearest neighbor; kernel density estimation; support vector machines; hidden Markov models; Boltzmann Learning; and classifiers may be combined either simply or in ways which minimize particular objective functions. For a review, see, e.g., Pattern Classification, R. O. Duda, et al., editors, John Wiley & Sons, 2nd edition, 2001; see also, The Elements of Statistical Learning—Data Mining, Inference, and Prediction, T. Hastie, et al., editors, Springer Science+Business Media, LLC, 2nd edition, 2009; each of which is incorporated by reference in its entirety.

To produce a classifier using supervised learning techniques, a set of samples called training data are obtained. In the context of prognostic tests, training data includes samples from the distinct groups (classes) to which unknown samples will later be assigned. For example, samples collected from individuals in a control population and individuals in a particular disease population can constitute training data to develop a classifier that can classify unknown samples (or, more particularly, the individuals from whom the samples were obtained) as either having the disease or being free from the disease. The development of the classifier from the training data is known as training the classifier. Specific details on classifier training depend on the nature of the supervised learning technique. For purposes of illustration, an example of training a random forest classifier will be described below (see, e.g., Pattern Classification, R. O. Duda, et al., editors, John Wiley & Sons, 2nd edition, 2001; see also, The Elements of Statistical Learning—Data Mining, Inference, and Prediction, T. Hastie, et al., editors, Springer Science+Business Media, LLC, 2nd edition, 2009).

Since typically there are many more potential biomarker values than samples in a training set, care must be used to avoid over-fitting. Over-fitting occurs when a statistical model describes random error or noise instead of the underlying relationship. Over-fitting can be avoided in a variety of ways, including, for example, by limiting the number of markers used in developing the classifier, by assuming that the marker responses are independent of one another, by limiting the complexity of the underlying statistical model employed, and by ensuring that the underlying statistical model conforms to the data.

An illustrative example of the development of an evaluation test using a set of biomarkers includes the application of a random forest classifier (Tao Shi and Steve Horvath (2006) Unsupervised Learning with Random Forest Predictors. Journal of Computational and Graphical Statistics. Volume 15, Number 1, March 2006, pp. 118-138(21). A RF predictor is an ensemble of individual classification tree predictors (Breiman, L. (2001) “Random forests”, Machine Learning, 45(1), 5-32). For each observation, each individual tree votes for one class and the forest predicts the class that has the plurality of votes. The user has to specify the number of randomly selected variables (mtry) to be searched through for the best split at each node. The Gini index (Breiman, L., Friedman, J. H., Olshen, R. A., Stone, C. J. (1984), Classification and Regression Trees, Chapman and Hall, New York.) is used as the splitting criterion. The largest tree possible is grown and is not pruned. The root node of each tree in the forest contains a bootstrap sample from the original data as the training set. The observations that are not in the training set, roughly ⅓ of the original data set, are referred to as out-of-bag (OOB) observations. One can arrive at OOB predictions as follows: for a case in the original data, predict the outcome by plurality vote involving only those trees that did not contain the case in their corresponding bootstrap sample. By contrasting these OOB predictions with the training set outcomes, one can arrive at an estimate of the prediction error rate, which is referred to as the OOB error rate.

Each biomarker is described by a class-dependent probability density function (pdf) for the measured RFU values or log RFU (relative fluorescence units) values in each class. The joint pdfs for the set of markers in one class is assumed to be the product of the individual class-dependent pdfs for each biomarker. Any underlying model for the class-dependent pdfs may be used, but the model should generally conform to the data observed in the training set.

The performance of the random forest classifier is dependent upon the number and quality of the biomarkers used to construct and train the classifier. A single biomarker will perform in accordance with its KS-distance (Kolmogorov-Smirnov) and its PCA value as exemplified herein] If a classifier performance metric is defined as the sum of the sensitivity (fraction of true positives, f_{TP}) and specificity (one minus the fraction of false positives, 1−f_{FP}), a perfect classifier will have a score of two and a random classifier, on average, will have a score of one. Using the definition of the KS-distance, that value x* which maximizes the difference in the cdf functions can be found by solving

$$\frac{\partial KS}{\partial x} = \frac{\partial ( {cdf}_{c} ( x ) - {cdf}_{d} ( x ) )}{\partial x} = 0$$

for x which leads to p(x*|c)=p(x*|d), i.e., the KS distance occurs where the class-dependent pdfs cross. Substituting this value of x* into the expression for the KS-distance yields the following definition for KS

$$\begin{matrix}KS = {cdf}_{c} ( {x}^{*} ) - {cdf}_{d} ( {x}^{*} ) \\ = {\int }_{- ∞}^{{x}^{*}} p ( x \vert c ) d x - {\int }_{- ∞}^{{x}^{*}} p ( x \vert d ) d x \\ = 1 - {\int }_{{x}^{*}}^{∞} p ( x \vert c ) d x - {\int }_{- ∞}^{{x}^{*}} p ( x \vert d ) d x \\ = 1 - {f}_{FP} - {f}_{FN} ,\end{matrix}$$

the KS distance is one minus the total fraction of errors using a test with a cut-off at x*, essentially a single analyte Bayesian classifier. Since we define a score of sensitivity+specificity=2−f_{FP}−f_{FN}, combining the above definition of the KS-distance we see that sensitivity+specificity=1+KS. We select biomarkers with a statistic that is inherently suited for building classifiers.

The addition of subsequent markers with good KS distances (>0.3, for example) will, in general, improve the classification performance if the subsequently added markers are independent of the first marker. Using the sensitivity plus specificity as a classifier score, it is straightforward to generate many high scoring classifiers.

Another way to identify relevant biomarkers is through Principal Components Analysis (PCA). PCA is a method that reduces data dimensionality by performing a covariance analysis between factors. As such, it is suitable for data sets in multiple dimensions, such as a large experiment in protein or gene expression. PCA uses an orthogonal transformation to convert a set of observations of possibly correlated variables into a set of values of uncorrelated variables called principal components. It is used as a tool in exploratory data analysis and for making predictive models. The central idea of principal component analysis (PCA) is to reduce the dimensionality of a data set consisting of a large number of interrelated variables, while retaining as much as possible of the variation present in the data set. This is achieved by transforming to a new set of variables, the principal components (PCs), which are uncorrelated, and which are ordered so that the first few retain most of the variation present in all of the original variables (Joliffe I T. (2002) Principal Component Analysis, 2^{nd} Edition. Springer).

Another way to depict classifier performance is through a receiver operating characteristic (ROC), or simply ROC curve. The ROC is a graphical plot of the sensitivity, or true positive rate, vs. false positive rate (1—specificity or 1—true negative rate), for a binary classifier system as its discrimination threshold is varied. The ROC can also be represented equivalently by plotting the fraction of true positives out of the positives (TPR=true positive rate) vs. the fraction of false positives out of the negatives (FPR=false positive rate). Also known as a Relative Operating Characteristic curve, because it is a comparison of two operating characteristics (TPR & FPR) as the criterion changes. The area under the ROC curve (AUC) is commonly used as a summary measure of diagnostic accuracy. It can take values from 0.0 to 1.0. The AUC has an important statistical property: the AUC of a classifier is equivalent to the probability that the classifier will rank a randomly chosen positive instance higher than a randomly chosen negative instance (Fawcett T, 2006. An introduction to ROC analysis. Pattern Recognition Letters. 27: 861-874). This is equivalent to the Wilcoxon test of ranks (Hanley, J. A., McNeil, B. J., 1982. The meaning and use of the area under a receiver operating characteristic (ROC) curve. Radiology 143, 29-36.).

The algorithm approach used here is exemplified herein. Briefly, all single analyte classifiers are generated from a table of potential biomarkers and added to a list. Next, all possible additions of a second analyte to each of the stored single analyte classifiers is then performed, saving a predetermined number of the best scoring pairs, say, for example, a thousand, on a new list. All possible three-marker classifiers are explored using this new list of the best two-marker classifiers, again saving the best thousand of these. This process continues until the score either plateaus or begins to deteriorate as additional markers are added. Those high scoring classifiers that remain after convergence can be evaluated for the desired performance for an intended use. For example, in one prognostic application, classifiers with a high sensitivity and modest specificity may be more desirable than modest sensitivity and high specificity. In another prognostic application, classifiers with a high specificity and a modest sensitivity may be more desirable. The desired level of performance is generally selected based upon a trade-off that must be made between the number of false positives and false negatives that can each be tolerated for the particular prognostic application. Such trade-offs generally depend on the medical consequences of an error, either false positive or false negative.

Various other techniques are known in the art and may be employed to generate many potential classifiers from a list of biomarkers using a random forest classifier. In one embodiment, what is referred to as a genetic algorithm can be used to combine different markers using the fitness score as defined above. Genetic algorithms are particularly well suited to exploring a large diverse population of potential classifiers. In another embodiment, so-called ant colony optimization can be used to generate sets of classifiers. Other strategies that are known in the art can also be employed, including, for example, other evolutionary strategies as well as simulated annealing and other stochastic search methods. Metaheuristic methods, such as, for example, harmony search may also be employed.

An illustrative example of the development of a diagnostic test using a set of biomarkers includes the application of a naïve Bayes classifier, a simple probabilistic classifier based on Bayes theorem with strict independent treatment of the biomarkers. Each biomarker is described by a class-dependent probability density function (pdf) for the measured RFU values or log RFU (relative fluorescence units) values in each class. The joint pdfs for the set of markers in one class is assumed to be the product of the individual class-dependent pdfs for each biomarker. Training a naïve Bayes classifier in this context amounts to assigning parameters (“parameterization”) to characterize the class dependent pdfs. Any underlying model for the class-dependent pdfs may be used, but the model should generally conform to the data observed in the training set.

Specifically, the class-dependent probability of measuring a value xᵢ for biomarker i in the disease class is written as p(xᵢ|d) and the overall naïve Bayes probability of observing n markers with values \tilde{x}=(x₁, x₂, . . . xₙ) is written as

$$\tilde{p} ( x \vert d ) = {\prod }_{i = 1}^{n} p ( {x}_{i} \vert d )$$

where the individual xᵢs are the measured biomarker levels in RFU or log RFU. The classification assignment for an unknown is facilitated by calculating the probability of being diseased p(\tilde{d}|x) having measured \tilde{x} compared to the probability of being disease free (control) p(\tilde{c}|x) for the same measured values. The ratio of these probabilities is computed from the class-dependent pdfs by application of Bayes theorem, i.e.,

$$\frac{p ( d \vert \tilde{x} )}{p ( c \vert \tilde{x} )} = \frac{p ( \tilde{x} \vert d ) p ( d )}{p ( \tilde{x} \vert c ) p ( c )}$$

where p(d) is the prevalence of the disease in the population appropriate to the test. Taking the logarithm of both sides of this ratio and substituting the naïve Bayes class-dependent probabilities from above gives

$$ln ( \frac{p ( d \vert \tilde{x} )}{p ( c \vert \tilde{x} )} ) = {\sum }_{i = 1}^{n} \frac{p ( {x}_{i} \vert d )}{p ( {x}_{i} \vert c )} + ln ( \frac{p ( d )}{1 - p ( d )} ) .$$

This form is known as the log likelihood ratio and simply states that the log likelihood of being free of the particular disease versus having the disease and is primarily composed of the sum of individual log likelihood ratios of the n individual biomarkers. In its simplest form, an unknown sample (or, more particularly, the individual from whom the sample was obtained) is classified as being free of the disease if the above ratio is greater than zero and having the disease if the ratio is less than zero.

In one exemplary embodiment, the class-dependent biomarker pdfs p(xᵢ|c) and p(xᵢ|d) are assumed to be normal or log-normal distributions in the measured RFU values xᵢ, i.e.

$$p ( {x}_{i} \vert c ) = \frac{1}{\sqrt{2 π} {σ}_{c , i}} exp ( \frac{{( {x}_{i} - {μ}_{c , i} )}^{2}}{- 2 {σ}_{c , i}^{2}} ) ,$$

with a similar expression for p(xᵢ|d) with μ_{d} and σ_{d}. Parameterization of the model requires estimation of two parameters for each class-dependent pdf, a mean μ and a variance σ², from the training data. This may be accomplished in a number of ways, including, for example, by maximum likelihood estimates, by least-squares, and by any other methods known to one skilled in the art. Substituting the normal distributions for μ and σ into the log-likelihood ratio defined above gives the following expression:

Once a set of μs and σ²s have been defined for each pdf in each class from the training data and the disease prevalence in the population is specified, the Bayes classifier is fully determined and may be used to classify unknown samples with measured values \tilde{x}.

The performance of the naïve Bayes classifier is dependent upon the number and quality of the biomarkers used to construct and train the classifier. A single biomarker will perform in accordance with its KS-distance (Kolmogorov-Smirnov), as defined in above. If a classifier performance metric is defined as the area under the receiver operator characteristic curve (AUC), a perfect classifier will have a score of 1 and a random classifier, on average, will have a score of 0.5. The definition of the KS-distance between two sets A and B of sizes n and m is the value, D_{n,m}=supₓ|F_{A,n}(x)−F_{B,m}(x)|, which is the largest difference between two empirical cumulative distribution functions (cdf). The empirical cdf for a set A of n observations Xᵢ is defined as,

$${F}_{A , n} ( x ) = \frac{1}{n} {\sum }_{i = 1}^{n} {I}_{{X}_{i} \leq x} , where {I}_{{X}_{i} \leq x}$$

is the indicator function which is equal to 1 if Xᵢ<x and is otherwise equal to 0. By definition, this value is bounded between 0 and 1, where a KS-distance of 1 indicates that the empirical distributions do not overlap.

The addition of subsequent markers with good KS distances (>0.3, for example) will, in general, improve the classification performance if the subsequently added markers are independent of the first marker. Using the area under the ROC curve (AUC) as a classifier score, it is straightforward to generate many high scoring classifiers with a variation of a greedy algorithm. (A greedy algorithm is any algorithm that follows the problem solving metaheuristic of making the locally optimal choice at each stage with the hope of finding the global optimum.).

The greedy algorithm approach used here is described in detail in Example 11. Briefly, all single analyte classifiers are generated from a table of potential biomarkers and added to a list. Next, all possible additions of a second analyte to each of the stored single analyte classifiers is then performed, saving a predetermined number of the best scoring pairs, say, for example, a thousand, on a new list. All possible three marker classifiers are explored using this new list of the best two-marker classifiers, again saving the best thousand of these. This process continues until the score either plateaus or begins to deteriorate as additional markers are added. Those high scoring classifiers that remain after convergence can be evaluated for the desired performance for an intended use. For example, in one diagnostic application, classifiers with a high sensitivity and modest specificity may be more desirable than modest sensitivity and high specificity. In another diagnostic application, classifiers with a high specificity and a modest sensitivity may be more desirable. The desired level of performance is generally selected based upon a trade-off that must be made between the number of false positives and false negatives that can each be tolerated for the particular diagnostic application. Such trade-offs generally depend on the medical consequences of an error, either false positive or false negative.

Various other techniques are known in the art and may be employed to generate many potential classifiers from a list of biomarkers using a naïve Bayes classifier. In one embodiment, what is referred to as a genetic algorithm can be used to combine different markers using the fitness score as defined above. Genetic algorithms are particularly well suited to exploring a large diverse population of potential classifiers. In another embodiment, so-called ant colony optimization can be used to generate sets of classifiers. Other strategies that are known in the art can also be employed, including, for example, other evolutionary strategies as well as simulated annealing and other stochastic search methods. Metaheuristic methods, such as, for example, harmony search may also be employed.

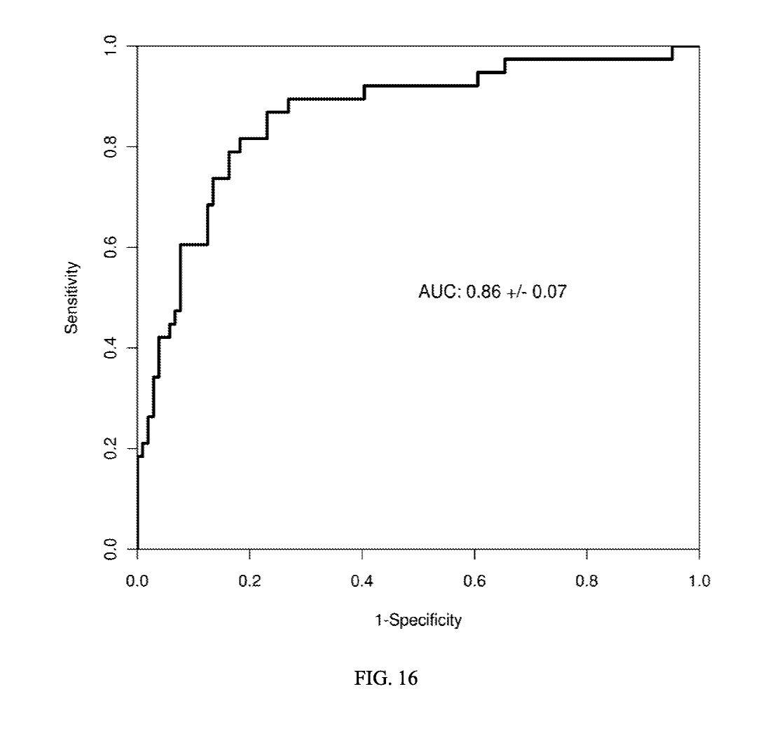

Exemplary embodiments use any number of the RCC biomarkers listed in Table 1 in various combinations to produce diagnostic tests for evaluating RCC (see Example 3 for a detailed description of how these biomarkers were identified). In one embodiment, a method for evaluating RCC uses a naïve Bayes classification method in conjunction with any number of the RCC biomarkers listed in Table 1. In an illustrative example (Example 11), the simplest test for prognosing RCC outcome of EVD from a population of individuals with an outcome of NED can be constructed using a single biomarker, for example, STC1 which is differentially expressed in the EVD vs. NED Outcome comparison with a KS-distance of 0.64. Using the parameters, μ_{c,i}, σ_{c,i}, μ_{d,i}, and σ_{d,i} for STC1 from Table 17 and the equation for the log-likelihood described above, a diagnostic test with an AUC of 0.862 can be derived, see Table 16. The ROC curve for this test is displayed in FIG. 16.

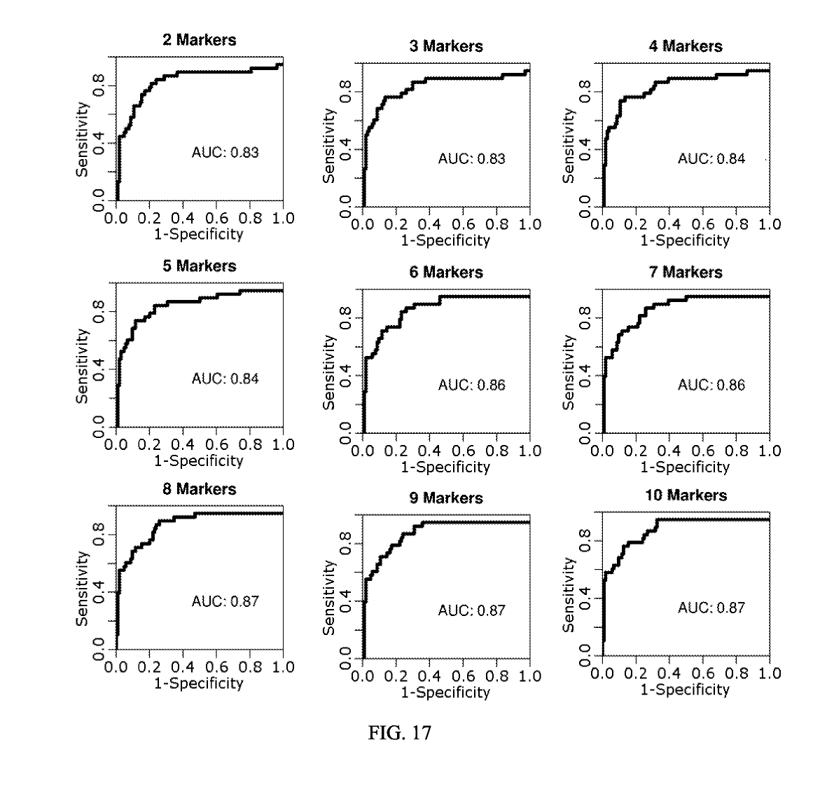

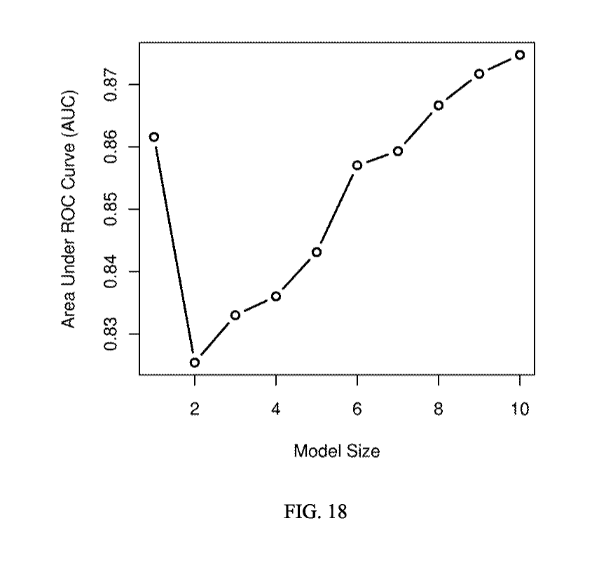

Addition of biomarker CXCL13, for example, with a KS-distance of 0.57, changes the classifier performance to an AUC of 0.825. Note that the score for a classifier constructed of two biomarkers is not a simple sum of the KS-distances; KS-distances are not additive when combining biomarkers and it takes many more weak markers to achieve the same level of performance as a strong marker. Adding a third marker, MMP7, for example, boosts the classifier performance to an AUC of 0.833. Adding additional biomarkers, such as, for example, RARRES2, HBA1-HBB, THBS4, TFPI, NTN4, CTSL2, and LDHB, produces a series of RCC tests summarized in Table 16 and displayed as a series of ROC curves in FIG. 17. The score of the classifiers as a function of the number of analytes used in classifier construction is displayed in FIG. 18. The AUC of this exemplary ten-marker classifier is 0.875.

The markers listed in Table 1 can be combined in many ways to produce classifiers for evaluating and diagnosing RCC. In some embodiments, panels of biomarkers are comprised of different numbers of analytes depending on a specific diagnostic performance criterion that is selected. For example, certain combinations of biomarkers will produce tests that are more sensitive (or more specific) than other combinations.

Once a panel is defined to include a particular set of biomarkers from Table 1 and a classifier is constructed from a set of training data, the definition of the diagnostic test is complete. The biological sample is appropriately diluted and then run in one or more assays to produce the relevant quantitative biomarker levels used for classification. The measured biomarker levels are used as input for the classification method that outputs a classification and an optional score for the sample that reflects the confidence of the class assignment.

Table 1 identifies 48 biomarkers that are useful for evaluating RCC. This is a surprisingly larger number than expected when compared to what is typically found during biomarker discovery efforts and may be attributable to the scale of the described study, which encompassed over 1030 proteins measured in hundreds of individual samples, in some cases at concentrations in the low femtomolar range. Presumably, the large number of discovered biomarkers reflects the diverse biochemical pathways implicated in both RCC biology and the body's response to RCC's presence; each pathway and process involves many proteins. The results show that no single protein of a small group of proteins is uniquely informative about such complex processes; rather, that multiple proteins are involved in relevant processes, such as apoptosis or extracellular matrix repair, for example.

Given the number of biomarkers identified during the described study, one would expect to be able to derive ample numbers of high-performing classifiers that can be used in various diagnostic methods. To test this notion, tens of thousands of classifiers were evaluated using the biomarkers in Table 1. As described in Example 11, many subsets of the biomarkers presented in Table 1 can be combined to generate useful classifiers. By way of example, descriptions are provided for classifiers containing 1, 2, and 3 biomarkers for evaluating RCC. As described in Example 10, all classifiers that were built using the biomarkers in Table 1 perform distinctly better than classifiers that were built using “non-markers”.

The performance of classifiers obtained by randomly excluding some of the markers in Table 1, which resulted in smaller subsets from which to build the classifiers, was also tested. As described in Example 11, Part 3, the classifiers that were built from random subsets of the markers in Table 1 performed similarly to optimal classifiers that were built using the full list of markers in Table 1.

The performance of ten-marker classifiers obtained by excluding the “best” individual markers from the ten-marker aggregation was also tested. As described in Example 11, classifiers constructed without the “best” markers in Table 1 also performed well. Many subsets of the biomarkers listed in Table 1 performed close to optimally, even after removing the top 15 of the markers listed in the Table. This implies that the performance characteristics of any particular classifier are likely not due to some small core group of biomarkers and that the disease process likely impacts numerous biochemical pathways, which alters the expression level of many proteins.

The results of classifier evaluation tests suggest certain possible conclusions: First, the identification of a large number of biomarkers enables their aggregation into a vast number of classifiers that offer similarly high performance. Second, classifiers can be constructed such that particular biomarkers may be substituted for other biomarkers in a manner that reflects the redundancies that undoubtedly pervade the complexities of the underlying disease processes. That is to say, the information about the disease contributed by any individual biomarker identified in Table 1 overlaps with the information contributed by other biomarkers, such that it may be that no particular biomarker or small group of biomarkers in Table 1 must be included in any classifier.

Exemplary embodiments use random forest and naive Bayes classifiers constructed from the data in Table 1 to classify an unknown sample. The procedure is outlined in FIGS. 1A and 1B. In one embodiment, the biological sample is optionally diluted and run in a multiplexed aptamer assay. The data from the assay are normalized and calibrated, and the resulting biomarker levels are used as input to a random forest or naive Bayes classification scheme as described in Examples 4 and 10. For the naive Bayes classifier, the log-likelihood ratio is computed for each measured biomarker individually and then summed to produce a final classification score, which is also referred to as a diagnostic score. The resulting assignment as well as the overall classification score can be reported. Optionally, the individual log-likelihood risk factors computed for each biomarker level can be reported as well. The details of the classification score calculation are presented in Example 11.

### Kits

Any combination of the biomarkers of Table 1 (as well as additional biomedical information) can be detected using a suitable kit, such as for use in performing the methods disclosed herein. Furthermore, any kit can contain one or more detectable labels as described herein, such as a fluorescent moiety, etc.

In one embodiment, a kit includes (a) one or more capture reagents (such as, for example, at least one aptamer or antibody) for detecting one or more biomarkers in a biological sample, wherein the biomarkers include any of the biomarkers set forth in Table 1, and optionally (b) one or more software or computer program products for classifying the individual from whom the biological sample was obtained, for evaluation of RCC status. Alternatively, rather than one or more computer program products, one or more instructions for manually performing the above steps by a human can be provided.

The combination of a solid support with a corresponding capture reagent and a signal generating material is referred to herein as a “detection device” or “kit”. The kit can also include instructions for using the devices and reagents, handling the sample, and analyzing the data. Further the kit may be used with a computer system or software to analyze and report the result of the analysis of the biological sample.

The kits can also contain one or more reagents (e.g., solubilization buffers, detergents, washes, or buffers) for processing a biological sample. Any of the kits described herein can also include, e.g., buffers, blocking agents, mass spectrometry matrix materials, antibody capture agents, positive control samples, negative control samples, software and information such as protocols, guidance and reference data.

In one aspect, the invention provides kits for the analysis of RCC status. The kits include PCR primers for aptamers specific to one or more biomarkers selected from Table 1. The kit may further include instructions for use and correlation of the biomarkers with RCC. The kit may also include any of the following, either alone or in combination: a DNA array containing the complement of aptamers to one or more of the biomarkers selected from Table 1, reagents, and enzymes for amplifying or isolating sample DNA. The kits may include reagents for real-time PCR, such as, for example, TaqMan probes and/or primers, and enzymes.

For example, a kit can comprise (a) reagents comprising at least capture reagents for quantifying one or more biomarkers in a test sample, wherein said biomarkers comprise the biomarkers set forth in Table 1, or any other biomarkers or biomarkers panels described herein, and optionally (b) one or more algorithms or computer programs for performing the steps of comparing the amount of each biomarker quantified in the test sample to one or more predetermined cutoffs and assigning a score for each biomarker quantified based on said comparison, combining the assigned scores for each biomarker quantified to obtain a total score, comparing the total score with a predetermined score, and using said comparison to evaluate RCC status in an individual. Alternatively, rather than one or more algorithms or computer programs, one or more instructions for manually performing the above steps by a human can be provided.

### Computer Methods and Software

Once a biomarker or biomarker panel is selected, a method for evaluating an individual for RCC status can comprise the following: 1) collect or otherwise obtain a biological sample; 2) perform an analytical method to detect and measure the biomarker or biomarkers in the panel in the biological sample; 3) perform any data normalization or standardization required for the method used to collect biomarker values; 4) calculate the marker score; 5) combine the marker scores to obtain a total diagnostic score; and 6) report the individual's diagnostic score. In this approach, the diagnostic score may be a single number determined from the sum of all the marker calculations that is compared to a preset threshold value that is an indication of the presence or absence of disease. Or the diagnostic score may be a series of bars that each represent a biomarker value and the pattern of the responses may be compared to a pre-set pattern for determination of the presence or absence of disease.

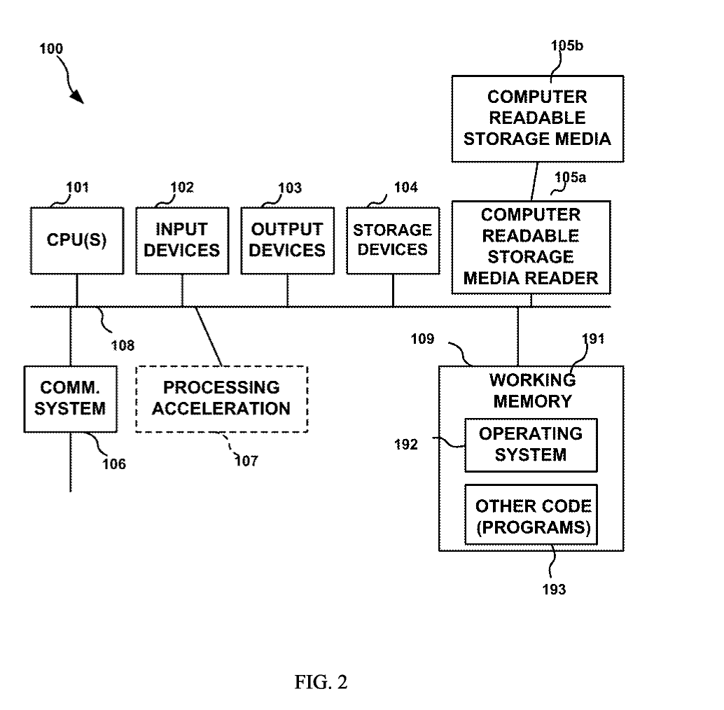

At least some embodiments of the methods described herein can be implemented with the use of a computer. An example of a computer system 100 is shown in FIG. 2. With reference to FIG. 2, system 100 is shown comprised of hardware elements that are electrically coupled via bus 108, including a processor 101, input device 102, output device 103, storage device 104, computer-readable storage media reader 105a, communications system 106 processing acceleration (e.g., DSP or special-purpose processors) 107 and memory 109. Computer-readable storage media reader 105a is further coupled to computer-readable storage media 105b, the combination comprehensively representing remote, local, fixed and/or removable storage devices plus storage media, memory, etc. for temporarily and/or more permanently containing computer-readable information, which can include storage device 104, memory 109 and/or any other such accessible system 100 resource. System 100 also comprises software elements (shown as being currently located within working memory 191) including an operating system 192 and other code 193, such as programs, data and the like.

With respect to FIG. 2, system 100 has extensive flexibility and configurability. Thus, for example, a single architecture might be utilized to implement one or more servers that can be further configured in accordance with currently desirable protocols, protocol variations, extensions, etc. However, it will be apparent to those skilled in the art that embodiments may well be utilized in accordance with more specific application requirements. For example, one or more system elements might be implemented as sub-elements within a system 100 component (e.g., within communications system 106). Customized hardware might also be utilized and/or particular elements might be implemented in hardware, software or both. Further, while connection to other computing devices such as network input/output devices (not shown) may be employed, it is to be understood that wired, wireless, modem, and/or other connection or connections to other computing devices might also be utilized.

In one aspect, the system can comprise a database containing features of biomarkers characteristic of RCC. The biomarker data (or biomarker information) can be utilized as an input to the computer for use as part of a computer implemented method. The biomarker data can include the data as described herein.

In one aspect, the system further comprises one or more devices for providing input data to the one or more processors.

The system further comprises a memory for storing a data set of ranked data elements.

In another aspect, the device for providing input data comprises a detector for detecting the characteristic of the data element, e.g., such as a mass spectrometer or gene chip reader.

The system additionally may comprise a database management system. User requests or queries can be formatted in an appropriate language understood by the database management system that processes the query to extract the relevant information from the database of training sets.

The system may be connectable to a network to which a network server and one or more clients are connected. The network may be a local area network (LAN) or a wide area network (WAN), as is known in the art. Preferably, the server includes the hardware necessary for running computer program products (e.g., software) to access database data for processing user requests.

The system may include an operating system (e.g., UNIX or Linux) for executing instructions from a database management system. In one aspect, the operating system can operate on a global communications network, such as the internet, and utilize a global communications network server to connect to such a network.

The system may include one or more devices that comprise a graphical display interface comprising interface elements such as buttons, pull down menus, scroll bars, fields for entering text, and the like as are routinely found in graphical user interfaces known in the art. Requests entered on a user interface can be transmitted to an application program in the system for formatting to search for relevant information in one or more of the system databases. Requests or queries entered by a user may be constructed in any suitable database language.

The graphical user interface may be generated by a graphical user interface code as part of the operating system and can be used to input data and/or to display inputted data. The result of processed data can be displayed in the interface, printed on a printer in communication with the system, saved in a memory device, and/or transmitted over the network or can be provided in the form of the computer readable medium.

The system can be in communication with an input device for providing data regarding data elements to the system (e.g., expression values). In one aspect, the input device can include a gene expression profiling system including, e.g., a mass spectrometer, gene chip or array reader, and the like.

The methods and apparatus for analyzing RCC biomarker information according to various embodiments may be implemented in any suitable manner, for example, using a computer program operating on a computer system. A conventional computer system comprising a processor and a random access memory, such as a remotely-accessible application server, network server, personal computer or workstation may be used. Additional computer system components may include memory devices or information storage systems, such as a mass storage system and a user interface, for example a conventional monitor, keyboard and tracking device. The computer system may be a stand-alone system or part of a network of computers including a server and one or more databases.

The RCC biomarker analysis system can provide functions and operations to complete data analysis, such as data gathering, processing, analysis, reporting and/or diagnosis. For example, in one embodiment, the computer system can execute the computer program that may receive, store, search, analyze, and report information relating to the RCC biomarkers. The computer program may comprise multiple modules performing various functions or operations, such as a processing module for processing raw data and generating supplemental data and an analysis module for analyzing raw data and supplemental data to generate a RCC status and/or diagnosis. Evaluating RCC status may comprise generating or collecting any other information, including additional biomedical information, regarding the condition of the individual relative to RCC, identifying whether further tests may be desirable, or otherwise evaluating the health status of the individual.

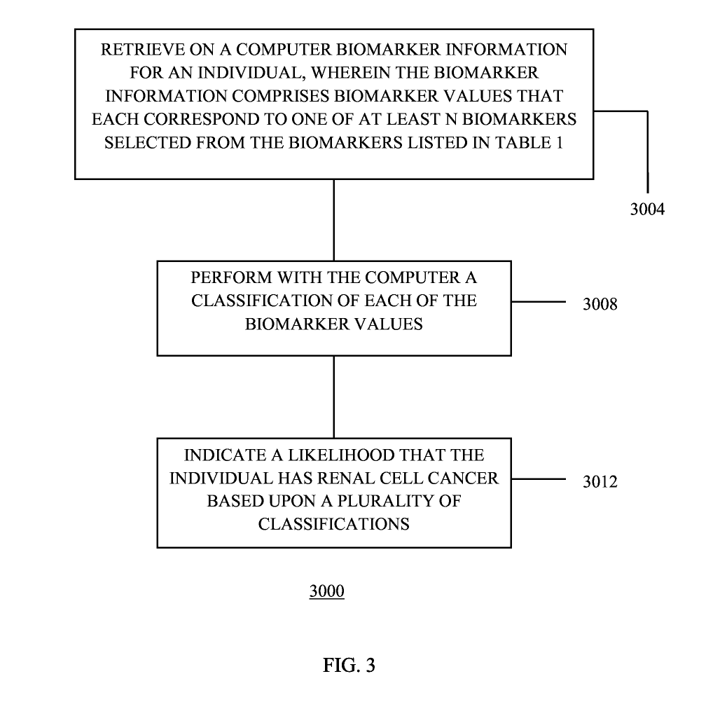

Referring now to FIG. 3 an example of a method of utilizing a computer in accordance with principles of a disclosed embodiment can be seen. In FIG. 3, a flowchart 3000 is shown. In block 3004, biomarker information can be retrieved for an individual. The biomarker information can be retrieved from a computer database, for example, after testing of the individual's biological sample is performed. The biomarker information can comprise biomarker values that each correspond to one of at least N biomarkers selected from a group consisting of the biomarkers provided in Table 1. In block 3008, a computer can be utilized to classify each of the biomarker values. And, in block 3012, an evaluation can be made regarding RCC status based upon a plurality of classifications. The indication can be output to a display or other indicating device so that it is viewable by a person. Thus, for example, it can be displayed on a display screen of a computer or other output device.

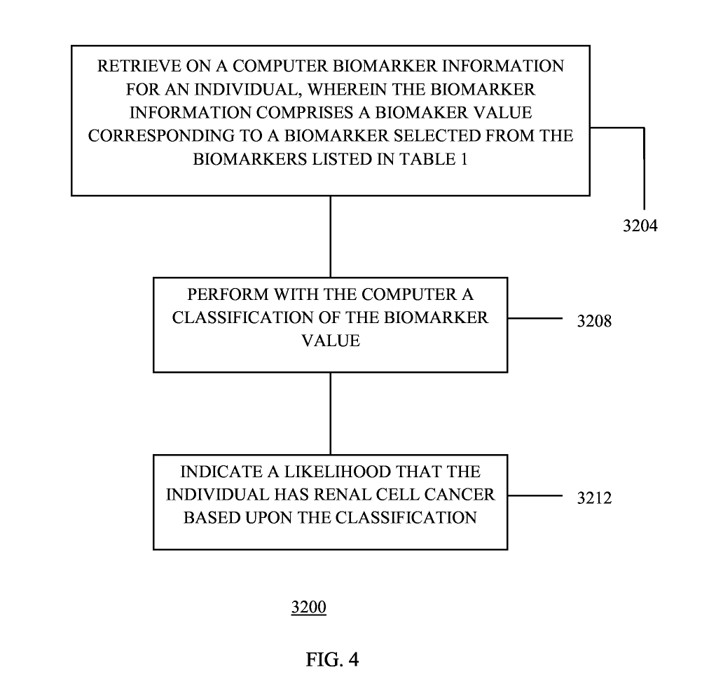

Referring now to FIG. 4, an alternative method of utilizing a computer in accordance with another embodiment can be illustrated via flowchart 3200. In block 3204, a computer can be utilized to retrieve biomarker information for an individual. The biomarker information comprises a biomarker value corresponding to a biomarker selected from the group of biomarkers provided in Table 1. In block 3208, a classification of the biomarker value can be performed with the computer. And, in block 3212, an indication can be made as to the RCC status of the individual based upon the classification. The indication can be output to a display or other indicating device so that it is viewable by a person. Thus, for example, it can be displayed on a display screen of a computer or other output device.

Some embodiments described herein can be implemented so as to include a computer program product. A computer program product may include a computer readable medium having computer readable program code embodied in the medium for causing an application program to execute on a computer with a database.

As used herein, a “computer program product” refers to an organized set of instructions in the form of natural or programming language statements that are contained on a physical media of any nature (e.g., written, electronic, magnetic, optical or otherwise) and that may be used with a computer or other automated data processing system. Such programming language statements, when executed by a computer or data processing system, cause the computer or data processing system to act in accordance with the particular content of the statements. Computer program products include without limitation: programs in source and object code and/or test or data libraries embedded in a computer readable medium. Furthermore, the computer program product that enables a computer system or data processing equipment device to act in pre-selected ways may be provided in a number of forms, including, but not limited to, original source code, assembly code, object code, machine language, encrypted or compressed versions of the foregoing and any and all equivalents.

In one aspect, a computer program product is provided for evaluating RCC status of an individual. The computer program product includes a computer readable medium embodying program code executable by a processor of a computing device or system, the program code comprising: code that retrieves data attributed to a biological sample from an individual, wherein the data comprises biomarker values that each correspond to one of at least N biomarkers in the biological sample selected from the group of biomarkers provided in Table 1; and code that executes a classification method that indicates RCC status of the individual as a function of the biomarker values.

In still another aspect, a computer program product is provided for evaluating RCC status. The computer program product includes a computer readable medium embodying program code executable by a processor of a computing device or system, the program code comprising: code that retrieves data attributed to a biological sample from an individual, wherein the data comprises a biomarker value corresponding to a biomarker in the biological sample selected from the group of biomarkers provided in Table 1; and code that executes a classification method that indicates a RCC disease status of the individual as a function of the biomarker value.

While various embodiments have been described as methods or apparatuses, it should be understood that embodiments can be implemented through code coupled with a computer, e.g., code resident on a computer or accessible by the computer. For example, software and databases could be utilized to implement many of the methods discussed above. Thus, in addition to embodiments accomplished by hardware, it is also noted that these embodiments can be accomplished through the use of an article of manufacture comprised of a computer usable medium having a computer readable program code embodied therein, which causes the enablement of the functions disclosed in this description. Therefore, it is desired that embodiments also be considered protected by this patent in their program code means as well. Furthermore, the embodiments may be embodied as code stored in a computer-readable memory of virtually any kind including, without limitation, RAM, ROM, magnetic media, optical media, or magneto-optical media. Even more generally, the embodiments could be implemented in software, or in hardware, or any combination thereof including, but not limited to, software running on a general purpose processor, microcode, PLAs, or ASICs.

It is also envisioned that embodiments could be accomplished as computer signals embodied in a carrier wave, as well as signals (e.g., electrical and optical) propagated through a transmission medium. Thus, the various types of information discussed above could be formatted in a structure, such as a data structure, and transmitted as an electrical signal through a transmission medium or stored on a computer readable medium.

It is also noted that many of the structures, materials, and acts recited herein can be recited as means for performing a function or step for performing a function. Therefore, it should be understood that such language is entitled to cover all such structures, materials, or acts disclosed within this specification and their equivalents, including the matter incorporated by reference.

### EXAMPLES

The following examples are provided for illustrative purposes only and are not intended to limit the scope of the application as defined by the appended claims. All examples described herein were carried out using standard techniques, which are well known and routine to those of skill in the art. Routine molecular biology techniques described in the following examples can be carried out as described in standard laboratory manuals, such as Sambrook et al., Molecular Cloning: A Laboratory Manual, 3rd. ed., Cold Spring Harbor Laboratory Press, Cold Spring Harbor, N.Y., (2001).

### Example 1

### Multiplexed Aptamer Analysis of Samples

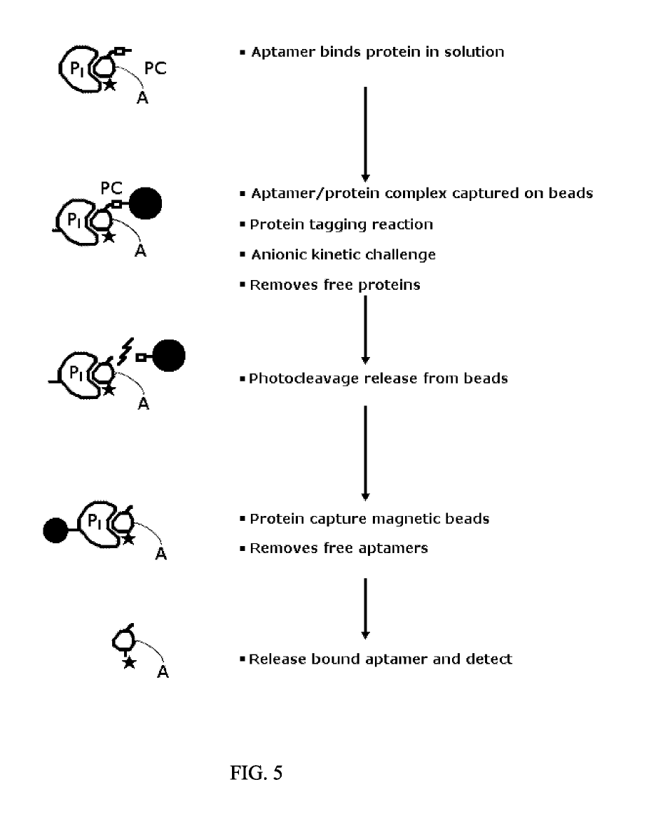

This example describes the multiplex aptamer assay used to analyze the cases and controls for the identification of the biomarkers set forth in Table 1. The SomaLogic proteomics discovery platform used in the studies presented herein (SOMAscan Version 2.0) measures ˜1030 proteins in blood from small sample volumes (˜15 uL of serum or plasma) with low limits of detection (1 pM average), ˜7 logs of overall dynamic range, and ˜5% average coefficient of variation. Proteins are measured with a process that transforms a signature of protein concentrations into a representative DNA concentration signature, which is quantified with a DNA microarray. See FIG. 5 for a brief description of the assay steps.

The subject invention comprises the use of “SOMAmers” or Slow-Off-rate Modified Aptamers. SOMAmers are single-stranded DNA nucleic acids that are modified to contain amino acid side chains, and have slow dissociation rates selected by kinetic challenge with a large excess of polyanionic competitor to remove non-specific polynucleotides. As a result, selected SOMAmers bind tightly to the target molecule—they are like high quality antibodies except that they are made out of nucleic acids instead of proteins.

The SomaLogic proteomics discovery platform is a multiplex proteomics assay (the assay), which measures proteins by transforming the quantity of a specific protein into an equivalent, or proportional, quantity of its cognate SOMAmer, which is captured in the assay and quantified by hybridization to a custom microarray.

A full description of the processes and performance of SOMAmer reagents and the SomaLogic multiplex proteomics assay is detailed in the publication: Gold L. et al. (2010) Aptamer-Based Multiplexed Proteomic Technology for Biomarker Discovery. PLoS ONE 5(12):e15004. doi:10.1371/journal.pone.0015004.

Abbreviations used herein include:

AUC: Area under the curve for ROC curve

BEN: Benign renal mass

DBV: Disease burden vector

EVD: Evidence of disease clinically

KS: Kolmogorov-Smirnov test

NDF: Never disease free, i.e., the patient never has complete clinical remission after surgery/treatment for RCC

NED: No evidence of disease, i.e., no clinical evidence of disease during follow up

PCA: Principal components analysis

REC: Recurrence of disease clinically

RFU: Relative fluorescence unit

ROC: Receiver operating characteristic

TP1: Timepoint 1, pre-surgery or pre-treatment

TP2: Timepoint 2, post-surgery or pre-treatment

Note: All SOMAmer targets are named by NCBI GeneID

In this method, pipette tips were changed for each solution addition.

Also, unless otherwise indicated, most solution transfers and wash additions used the 96-well head of a Beckman Biomek FxP. Method steps manually pipetted used a twelve channel P200 Pipetteman (Rainin Instruments, LLC, Oakland, Calif.), unless otherwise indicated. A custom buffer referred to as SB17 was prepared in-house, comprising 40 mM HEPES, 100 mM NaCl, 5 mM KCl, 5 mM MgCl₂, 1 mM EDTA at pH 7.5. A custom buffer referred to as SB18 was prepared in-house, comprising 40 mM HEPES, 100 mM NaCl, 5 mM KCl, 5 mM MgCl₂ at pH 7.5. All steps were performed at room temperature unless otherwise indicated.

1. Preparation of Aptamer Stock Solution

Custom stock aptamer solutions for 5%, 0.316% and 0.01% serum were prepared at 2× concentration in 1×SB17, 0.05% Tween-20.

These solutions are stored at −20° C. until use. The day of the assay, each aptamer mix was thawed at 37° C. for 10 minutes, placed in a boiling water bath for 10 minutes and allowed to cool to 25° C. for 20 minutes with vigorous mixing in between each heating step. After heat-cool, 55 μL of each 2× aptamer mix was manually pipetted into a 96-well Hybaid plate and the plate foil sealed. The final result was three, 96-well, foil-sealed Hybaid plates with 5%, 0.316% or 0.01% aptamer mixes. The individual aptamer concentration was 2× final or 1 nM.

2. Assay Sample Preparation

Frozen aliquots of 100% serum or plasma, stored at −80° C., were placed in 25° C. water bath for 10 minutes. Thawed samples were placed on ice, gently vortexed (set on 4) for 8 seconds and then replaced on ice.

A 10% sample solution (2× final) was prepared by transferring 8 μL of sample using a 50 μL 8-channel spanning pipettor into 96-well Hybaid plates, each well containing 72 μL of the appropriate sample diluent at 4° C. (1×SB17 for serum or 0.8×SB18 for plasma, plus 0.06% Tween-20, 11.1 μM Z-block_{—}2, 0.44 mM MgCl₂, 2.2 mM AEBSF, 1.1 mM EGTA, 55.6 μM EDTA). This plate was stored on ice until the next sample dilution steps were initiated on the BiomekFxP robot.

To commence sample and aptamer equilibration, the 10% sample plate was briefly centrifuged and placed on the Beckman FX where it was mixed by pipetting up and down with the 96-well pipettor. A 0.632% sample plate (2× final) was then prepared by diluting 6 μL of the 10% sample into 89 μL of 1×SB17, 0.05% Tween-20 with 2 mM AEBSF. Next, dilution of 6 μL of the resultant 0.632% sample into 184 μL of 1×SB17, 0.05% Tween-20 made a 0.02% sample plate (2× final). Dilutions were done on the Beckman Biomek FxP. After each transfer, the solutions were mixed by pipetting up and down. The 3 sample dilution plates were then transferred to their respective aptamer solutions by adding 55 μL of the sample to 55 μL of the appropriate 2× aptamer mix. The sample and aptamer solutions were mixed on the robot by pipetting up and down.

3. Sample Equilibration Binding

The sample/aptamer plates were foil sealed and placed into a 37° C. incubator for 3.5 hours before proceeding to the Catch 1 step.

4. Preparation of Catch 2 Bead Plate

An 11 mL aliquot of MyOne (Invitrogen Corp., Carlsbad, Calif.) Streptavidin C1 beads (10 mg/mL) was washed 2 times with equal volumes of 20 mM NaOH (5 minute incubation for each wash), 3 times with equal volumes of 1×SB17, 0.05% Tween-20 and resuspended in 11 mL 1×SB17, 0.05% Tween-20. Using a 12-span multichannel pipettor, 50 μL of this solution was manually pipetted into each well of a 96-well Hybaid plate. The plate was then covered with foil and stored at 4° C. for use in the assay.

5. Preparation of Catch 1 Bead Plates

Three 0.45 μm Millipore HV plates (Durapore membrane, Cat# MAHVN4550) were equilibrated with 100 μL of 1×SB17, 0.05% Tween-20 for at least 10 minutes. The equilibration buffer was then filtered through the plate and 133.3 μL of a 7.5% streptavidin-agarose bead slurry (in 1×SB17, 0.05% Tween-20) was added into each well. To keep the streptavidin-agarose beads suspended while transferring them into the filter plate, the bead solution was manually mixed with a 200 μL, 12-channel pipettor, at least 6 times between pipetting events. After the beads were distributed across the 3 filter plates, a vacuum was applied to remove the bead supernatant. Finally, the beads were washed in the filter plates with 200 μL 1×SB17, 0.05% Tween-20 and then resuspended in 200 μL 1×SB17, 0.05% Tween-20. The bottoms of the filter plates were blotted and the plates stored for use in the assay.

6. Loading the Cytomat

The cytomat was loaded with all tips, plates, all reagents in troughs (except NHS-biotin reagent which was prepared fresh right before addition to the plates), 3 prepared Catch 1 filter plates and 1 prepared MyOne plate.

7. Catch 1

After a 3.5 hour equilibration time, the sample/aptamer plates were removed from the incubator, centrifuged for about 1 minute, cover removed, and placed on the deck of the Beckman Biomek FxP. The Beckman Biomek FxP program was initiated. All subsequent steps in Catch 1 were performed by the Beckman Biomek FxP robot unless otherwise noted. Within the program, the vacuum was applied to the Catch 1 filter plates to remove the bead supernatant. One hundred microlitres of each of the 5%, 0.316% and 0.01% equilibration binding reactions were added to their respective Catch 1 filtration plates, and each plate was mixed using an on-deck orbital shaker at 800 rpm for 10 minutes.

Unbound solution was removed via vacuum filtration. The Catch 1 beads were washed with 190 μL of 100 μM biotin in 1×SB 17, 0.05% Tween-20 followed by 5×190 μL of 1×SB17, 0.05% Tween-20 by dispensing the solution and immediately drawing a vacuum to filter the solution through the plate.

8. Tagging

A 100 mM NHS-PEO4-biotin aliquot in anhydrous DMSO was thawed at 37° C. for 6 minutes and then diluted 1:100 with tagging buffer (SB17 at pH 7.25, 0.05% Tween-20). Upon a robot prompt, the diluted NHS-PEO4-biotin reagent was manually added to an on-deck trough and the robot program was manually re-initiated to dispense 100 μL of the NHS-PEO4-biotin into each well of each Catch 1 filter plate. This solution was allowed to incubate with Catch 1 beads shaking at 800 rpm for 5 minutes on the orbital shakers.

9. Kinetic Challenge and Photo-Cleavage

The tagging reaction was removed by vacuum filtration and quenched by the addition of 150 μL of 20 mM glycine in 1×SB17, 0.05% Tween-20 to the Catch 1 plates. The NHS-tag/glycine solution was removed via vacuum filtration. Next, 1500 μL 20 mM glycine (1×SB17, 0.05% Tween-20) was added to each plate and incubated for 1 minute on orbital shakers at 800 rpm before removal by vacuum filtration.

The wells of the Catch 1 plates were subsequently washed three times by adding 190 μL 1×SB17, 0.05% Tween-20, followed by vacuum filtration and then by adding 190 μL 1×SB17, 0.05% Tween-20 with shaking for 1 minute at 800 rpm followed by vacuum filtration. After the last wash the plates were placed on top of a 1 mL deep-well plate and removed from the deck. The Catch 1 plates were centrifuged at 1000 rpm for 1 minute to remove as much extraneous volume from the agarose beads before elution as possible.

The plates were placed back onto the Beckman Biomek FxP and 85 μL of 10 mM DxSO4 in 1×SB17, 0.05% Tween-20 was added to each well of the filter plates.

The filter plates were removed from the deck, placed onto a Variomag Thermoshaker (Thermo Fisher Scientific, Inc., Waltham, Mass.) under the BlackRay (Ted Pella, Inc., Redding, Calif.) light sources, and irradiated for 5 minutes while shaking at 800 rpm. After the 5 minute incubation the plates were rotated 180 degrees and irradiated with shaking for 5 minutes more.

The photocleaved solutions were sequentially eluted from each Catch 1 plate into a common deep well plate by first placing the 5% Catch 1 filter plate on top of a 1 mL deep-well plate and centrifuging at 1000 rpm for 1 minute. The 0.316% and 0.01% Catch 1 plates were then sequentially centrifuged into the same deep well plate.

10. Catch 2 Bead Capture

The 1 mL deep well block containing the combined eluates of Catch 1 was placed on the deck of the Beckman Biomek FxP for Catch 2.

The robot transferred all of the photo-cleaved eluate from the 1 mL deep-well plate onto the Hybaid plate containing the previously prepared Catch 2 MyOne magnetic beads (after removal of the MyOne buffer via magnetic separation).

The solution was incubated while shaking at 1350 rpm for 5 minutes at 25° C. on a Variomag Thermoshaker (Thermo Fisher Scientific, Inc., Waltham, Mass.).

The robot transferred the plate to the on deck magnetic separator station. The plate was incubated on the magnet for 90 seconds before removal and discarding of the supernatant.

11. 37° C. 30% Glycerol Washes

The Catch 2 plate was moved to the on-deck thermal shaker and 75 μL of 1×SB17, 0.05% Tween-20 was transferred to each well. The plate was mixed for 1 minute at 1350 rpm and 37° C. to resuspend and warm the beads. To each well of the Catch 2 plate, 75 μL of 60% glycerol at 37° C. was transferred and the plate continued to mix for another minute at 1350 rpm and 37° C. The robot transferred the plate to the 37° C. magnetic separator where it was incubated on the magnet for 2 minutes and then the robot removed and discarded the supernatant. These washes were repeated two more times.

After removal of the third 30% glycerol wash from the Catch 2 beads, 150 μL of 1×SB17, 0.05% Tween-20 was added to each well and incubated at 37° C., shaking at 1350 rpm for 1 minute, before removal by magnetic separation on the 37° C. magnet.

The Catch 2 beads were washed a final time using 150 μL 1×SB17, 0.05% Tween-20 with incubation for 1 minute while shaking at 1350 rpm at 25° C. prior to magnetic separation.

12. Catch 2 Bead Elution and Neutralization

The aptamers were eluted from Catch 2 beads by adding 105 μL of 100 mM CAPSO with 1 M NaCl, 0.05% Tween-20 to each well. The beads were incubated with this solution with shaking at 1300 rpm for 5 minutes.

The Catch 2 plate was then placed onto the magnetic separator for 90 seconds prior to transferring 63 μL of the eluate to a new 96-well plate containing 7 μL of 500 mM HCl, 500 mM HEPES, 0.05% Tween-20 in each well. After transfer, the solution was mixed robotically by pipetting 60 μL up and down five times.

13. Hybridization

The Beckman Biomek FxP transferred 20 μL of the neutralized Catch 2 eluate to a fresh Hybaid plate, and 6 μL of 10× Agilent Block, containing a 10× spike of hybridization controls, was added to each well. Next, 30 μL of 2× Agilent Hybridization buffer was manually pipetted to the each well of the plate containing the neutralized samples and blocking buffer and the solution was mixed by manually pipetting 25 μL up and down 15 times slowly to avoid extensive bubble formation. The plate was spun at 1000 rpm for 1 minute.

Custom Agilent microarray slides (Agilent Technologies, Inc., Santa Clara, Calif.) were designed to contain probes complementary to the aptamer random region plus some primer region. For the majority of the aptamers, the optimal length of the complementary sequence was empirically determined and ranged between 40-50 nucleotides. For later aptamers a 46-mer complementary region was chosen by default. The probes were linked to the slide surface with a poly-T linker for a total probe length of 60 nucleotides.

A gasket slide was placed into an Agilent hybridization chamber and 40 μL of each of the samples containing hybridization and blocking solution was manually pipetted into each gasket. An 8-channel variable spanning pipettor was used in a manner intended to minimize bubble formation. Custom Agilent microarray slides (Agilent Technologies, Inc., Santa Clara, Calif.), with their Number Barcode facing up, were then slowly lowered onto the gasket slides (see Agilent manual for detailed description).

The top of the hybridization chambers were placed onto the slide/backing sandwich and clamping brackets slid over the whole assembly. These assemblies were tightly clamped by turning the screws securely.

Each slide/backing slide sandwich was visually inspected to assure the solution bubble could move freely within the sample. If the bubble did not move freely the hybridization chamber assembly was gently tapped to disengage bubbles lodged near the gasket.

The assembled hybridization chambers were incubated in an Agilent hybridization oven for 19 hours at 60° C. rotating at 20 rpm.

14. Post Hybridization Washing

Approximately 400 mL Agilent Wash Buffer 1 was placed into each of two separate glass staining dishes. One of the staining dishes was placed on a magnetic stir plate and a slide rack and stir bar were placed into the buffer.

A staining dish for Agilent Wash 2 was prepared by placing a stir bar into an empty glass staining dish.

A fourth glass staining dish was set aside for the final acetonitrile wash.

Each of six hybridization chambers was disassembled. One-by-one, the slide/backing sandwich was removed from its hybridization chamber and submerged into the staining dish containing Wash 1. The slide/backing sandwich was pried apart using a pair of tweezers, while still submerging the microarray slide. The slide was quickly transferred into the slide rack in the Wash 1 staining dish on the magnetic stir plate.

The slide rack was gently raised and lowered 5 times. The magnetic stirrer was turned on at a low setting and the slides incubated for 5 minutes.

When one minute was remaining for Wash 1, Wash Buffer 2 pre-warmed to 37° C. in an incubator was added to the second prepared staining dish. The slide rack was quickly transferred to Wash Buffer 2 and any excess buffer on the bottom of the rack was removed by scraping it on the top of the stain dish. The slide rack was gently raised and lowered 5 times. The magnetic stirrer was turned on at a low setting and the slides incubated for 5 minutes.

The slide rack was slowly pulled out of Wash 2, taking approximately 15 seconds to remove the slides from the solution.

With one minute remaining in Wash 2 acetonitrile (ACN) was added to the fourth staining dish. The slide rack was transferred to the acetonitrile stain dish. The slide rack was gently raised and lowered 5 times. The magnetic stirrer was turned on at a low setting and the slides incubated for 5 minutes.

The slide rack was slowly pulled out of the ACN stain dish and placed on an absorbent towel. The bottom edges of the slides were quickly dried and the slide was placed into a clean slide box.

15. Microarray Imaging

The microarray slides were placed into Agilent scanner slide holders and loaded into the Agilent Microarray scanner according to the manufacturer Â's instructions.

The slides were imaged in the Cy3-channel at 5 μm resolution at the 100% PMT setting and the XRD option enabled at 0.05. The resulting tiff images were processed using Agilent feature extraction software version 10.5.

### Example 2

### Study Design

The specific intended clinical applications for the subject SOMAmers are:

1. Pre-surgical or pre-treatment prediction of prognosis

2. Monitoring of post-resection or post-treatment residual disease and recurrence

3. Differential diagnosis of renal mass as BEN or RCC; and

4. Determination of disease burden in an RCC patient, either at diagnosis or during post-treatment monitoring.

To support these applications, a prospectively designed case-control study was performed on retrospective serum samples obtained from renal cell carcinoma patients (RCC) and benign renal mass controls (BEN). Pre-surgical samples (TP1) were obtained for all subjects. A single post-surgical serum sample (TP2) was available for a subset of these subjects. A total of 385 samples were available for analysis; 75% were used in training and 25% were withheld as a blinded verification set. The results were unblinded by an independent 3rd party statistician.

The primary analysis compared outcome data as recorded in the SEER database field CA Status 1 (“SEER”=Surveillance, Epidemiology and End Results program at NCI for reporting US cancer statistics) for the RCC patients with “Evidence of Disease” (EVD) vs. “No Evidence of Disease” (NED) documented through clinical follow-up. Biomarkers were discovered in pre-surgical TP1 samples and a random forest classifier was developed for Outcome with an AUC of 0.9, which provides prognostic information prior to surgical resection and may be useful for monitoring post-surgical recurrence.

Although the number of EVD and recurred subjects is small in the post-surgical T2 sample set, the distribution of biomarkers is consistent with clinical outcome and recurrence data.

All serum samples were collected after obtaining informed consent. Samples were collected in red top serum tubes by trained biorepository staff and stored at −80° C. Samples have been thawed once for aliquoting and once for the assay.

All TP1 specimens were collected prior to treatment. TP2 samples were collected a median of 16 days post-surgery (range 4-1195 days). A total of 385 samples from 173 subjects were available for analysis.

### Training Set Cohort

The diagnosis and demographic distribution of the training set are detailed in Tables 2-5. Age and gender are balanced between the two critical training groups, NED and EVD. There are a higher proportion of males in the BEN group, but this group was not used in biomarker selection or prognosis classifier training. The BEN subjects were used to confirm that the distribution of markers in the NED group was consistent in individuals with non-malignant diagnoses and to derive the differential diagnosis classifier of benign renal mass vs. Stage II-IV RCC.

Pathologic stage is an important predictor of clinical outcome and disease recurrence. However, as seen in Tables 4 and 5, stage is not a perfect measure of outcome. Note that there are early stage (I and II) subjects with EVD, and late stage (III and IV) with NED.

All major histological categories are represented in the cases and controls. Cases and controls are categorized as RCC positive or negative based on pathological diagnosis. The RCC cases include clear cell, chromophobe, papillary, transitional cell, and sarcomatoid histologies. The benign diagnoses include renal cyst, renal mass, angiomyolipoma and oncocytoma.

Median clinical follow-up for the determination of NED or EVD was 773 days (range 10-2137 days). At least one year follow-up was available for 116 (82%) of the RCC subjects. Of the subjects in the EVD group, 7 had documented recurrence and the remainder were never disease free.

### Example 3

### Biomarker Identification

Biomarkers Associated with Outcome

Two complementary approaches were used to identify biomarkers associated with outcome (EVD vs. NED) in TP1 samples: the Kolmogorov-Smirnov test (KS test) and Principal Components Analysis (PCA). Univariate analysis was performed using the non-parametric KS statistic, which quantifies a distance between the cumulative distribution function of each SOMAmer for two reference distributions designated case (EVD) and control (NED). PCA is a multivariate approach to convert a set of observations of possibly correlated variables into a set of values of uncorrelated variables called principal components. The result is a principal component composed of covarying SOMAmers that correlate with the case/control division of samples.

After identifying potential biomarkers with both the KS test and PCA, backwards selection was performed to generate a random forest classifier with an AUC of 0.9 for RCC Outcome (Outcome model). The details of both of these biomarker discovery processes are described below.

Biomarkers Identified Through KS Test Analysis

Each of the case and control populations were separately compared by generating class-dependent cumulative distribution functions (cdfs) for each of the 1045 analytes. The KS-distance (Kolmogorov-Smirnov statistic) between values from two sets of samples is a non parametric measurement of the extent to which the empirical distribution of the values from one set (Set A) differs from the distribution of values from the other set (Set B). For any value of a threshold T some proportion of the values from Set A will be less than T, and some proportion of the values from Set B will be less than T. The KS-distance measures the maximum (unsigned) difference between the proportion of the values from the two sets for any choice of T. Univariate analysis using the KS test identified 98 biomarkers with a q-value (false discovery rate corrected p-value) less than 0.01 and 43 markers with a q-value<0.001. The KS statistic varies between zero (no difference in distribution, not a biomarker) and one (no overlap in distribution, a perfect biomarker).

Biomarkers Identified Through Multivariate PCA Analysis'

PCA analysis revealed a major component (PC1) that separated samples based on EVD vs. NED Outcome

A large set of markers, including 186 up-regulated and 76 down-regulated SOMAmers, was assembled for backwards selection. Although CRP (C Reactive Protein) and SAA (Serum Amyloid A) showed strong correlation with outcome, these acute phase reactants were excluded from final biomarker selection because they are nonspecific indicators of inflammation. The resulting biomarkers from both the univariate and multivariate approaches are shown in Table 1. This set of potential biomarkers can be used to build classifiers that assign samples to either a control or a disease group. In fact, many such classifiers were produced from these sets of biomarkers and the frequency with which any biomarker was used in good scoring classifiers determined. Those biomarkers that occurred most frequently among the top scoring classifiers were the most useful for creating a diagnostic test. Example 4 describes a random forest classifier and Example 11 describes Bayesian classifiers that were used to explore the classification space, but many other supervised learning techniques may be employed for this purpose. The scoring fitness of any individual classifier was gauged by the area under the receiver operating characteristic curve (AUC of the ROC) of the classifier at the Bayesian surface assuming a disease prevalence of 0.5. This scoring metric varies from zero to one, with one being an error-free classifier.

### Example 4

### Training a Random Forest Classifier for Clinical Outcome

Random forest (RF) classifiers were separately derived by backwards selection from biomarkers identified by the KS test or PCA analysis. Each analysis resulted in models containing 16 proteins, 12 of which are in common between the two approaches (Table 6).

The case/control distributions of these 20 markers were examined, and the most promising for consistent differential expression in TP1 Outcome, TP2 Outcome, and TP1 Stage were used in backwards selection to generate a RF model for Outcome. The AUC ranges for the Outcome models ranged from 0.77 to 0.89 and contained 1-15 markers. A 10-biomarker classifier with an AUC of 0.89 was chosen for further analysis. The markers and Gini importance scores (Gini is a measure of the purity of a set of classes) are shown in Table 7.

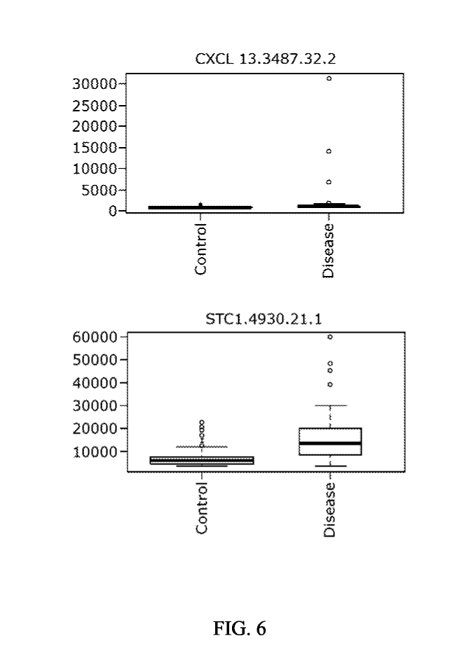

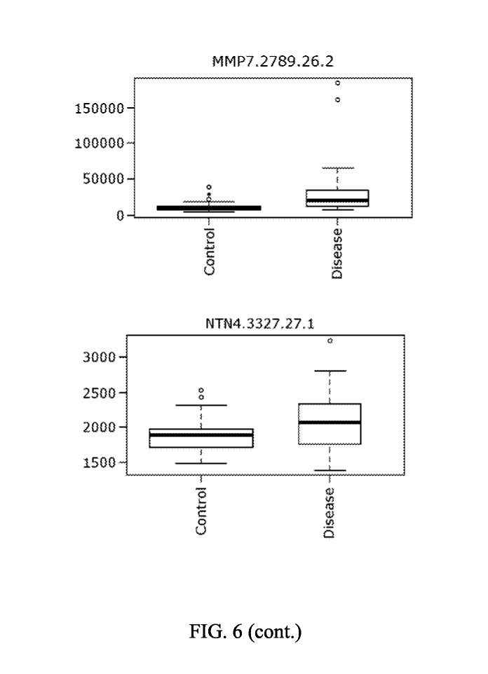

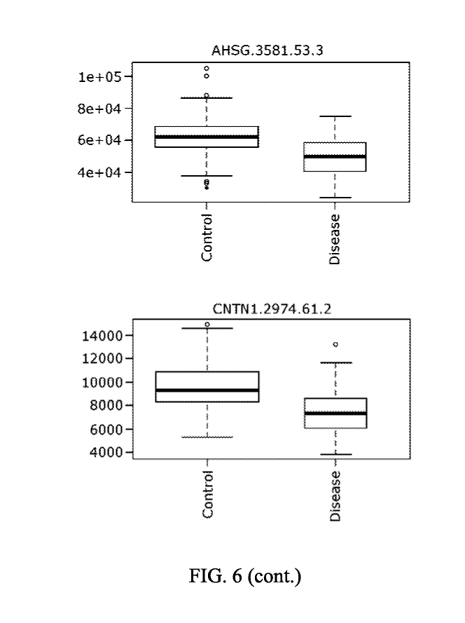

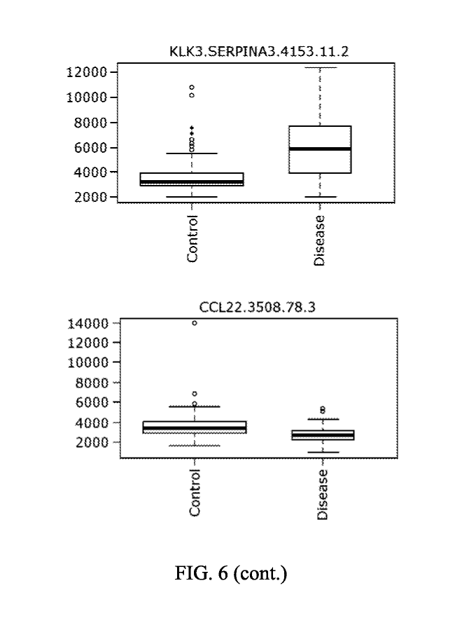

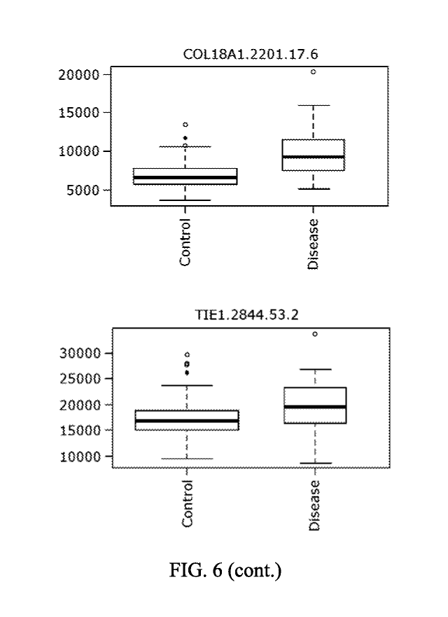

The distribution of these biomarkers in TP1 Outcome is shown as boxplots in FIG. 6. Of the 10 SOMAmers, 7 are up-regulated and 3 are down-regulated in EVD vs. NED.

### Example 5

### Correlation of the Outcome Classifier with RCC Pathologic Stage

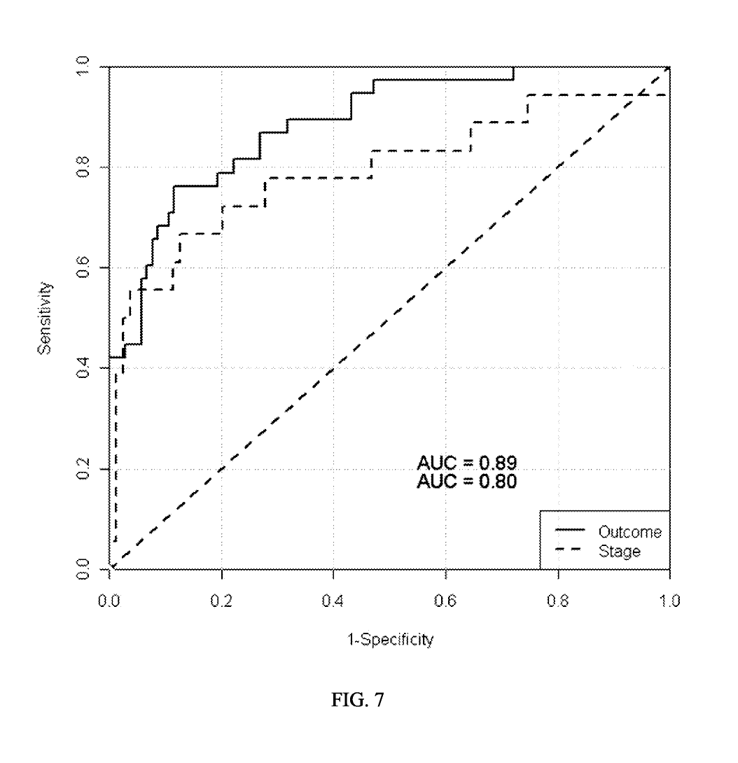

The Outcome model was trained on NED vs. EVD, which often correlates with pathologic stage, as can be seen in Table 4. To check for consistency with pathologic stage, we tested this model on Stage I vs. III. As can be seen in FIG. 7, the Outcome model has an AUC of 0.8 when tested against early vs. late stage disease, confirming that this model correlates with Stage, the most reliable predictor of outcome available today. The model provides additional prognostic input prior to surgery which may guide neoadjuvant or surgical treatment choices. However, pathologic stage is not a perfect predictor; there are early stage disease patients with EVD and late stage patients with NED after resection. Our model correctly predicts the observed outcome for many of these patients. The model also correctly classifies all BEN subjects as NED. Since the BEN subjects were not included in the training set, these results are an independent verification of the specificity of the model for EVD.

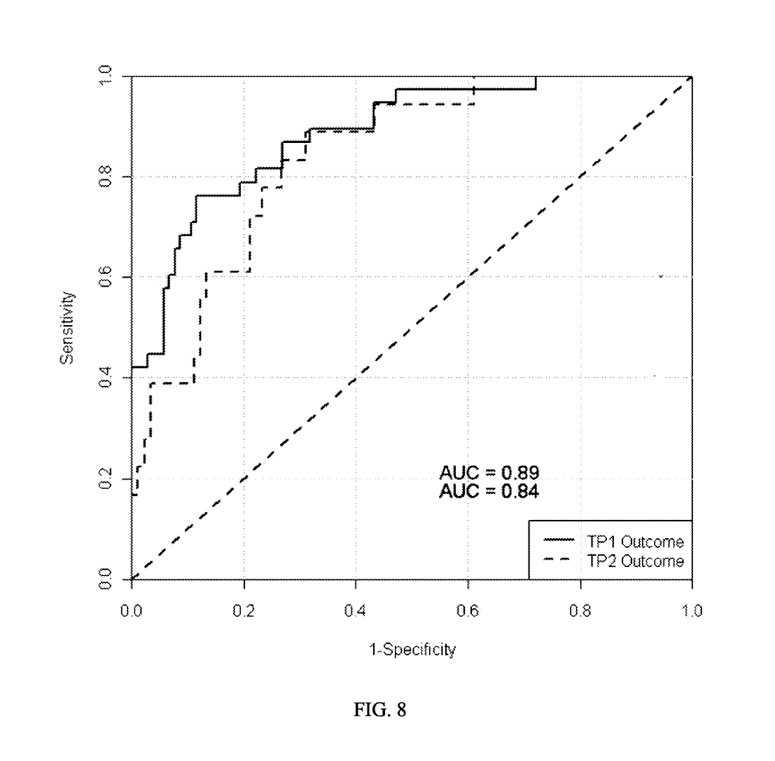

We tested the persistence of the prognostic power of this model in the available post-surgical TP2 samples (FIG. 8). The model works well in TP2 post-surgical samples, with an AUC of 0.84, supporting a potential use in monitoring disease progression or recurrence.

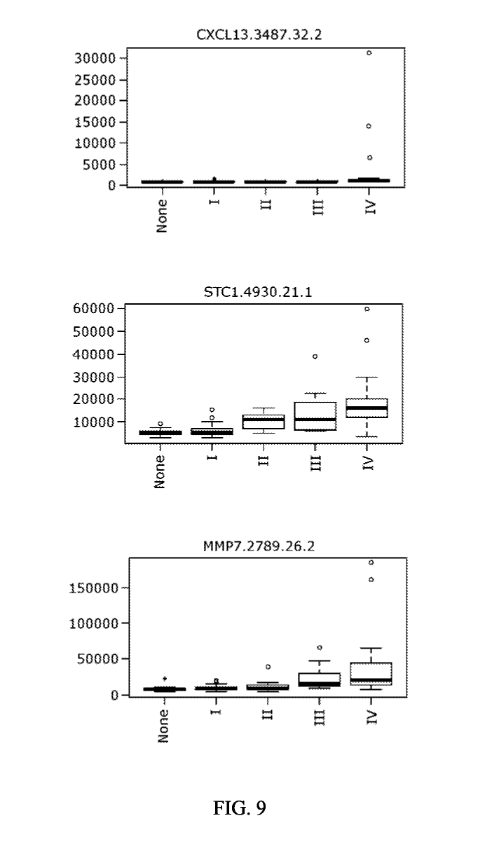

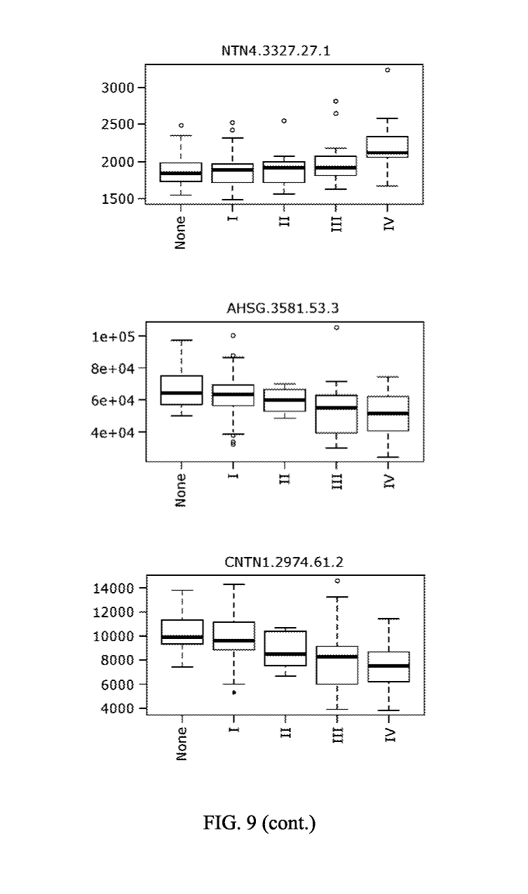

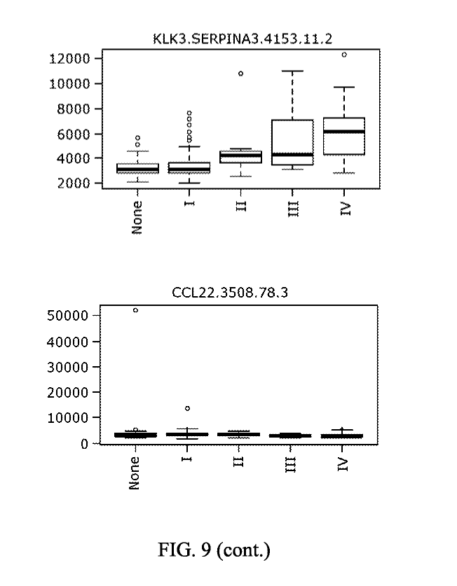

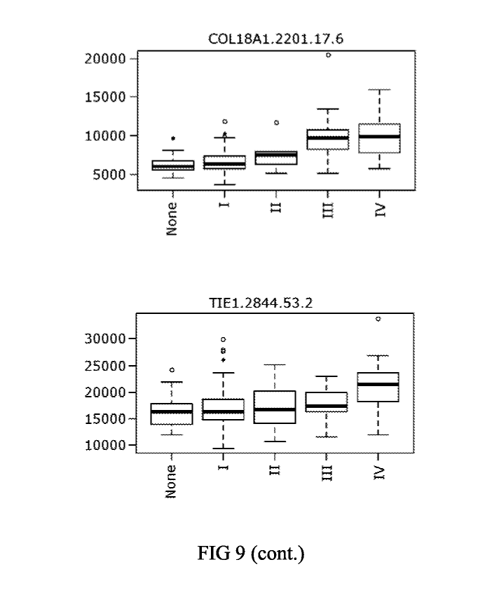

Since pathologic stage is an indicator of pre-surgery extent of disease, we examined the distribution of the 10 SOMAmer measurements by Stage (FIG. 9). The levels of both the up-regulated and down-regulated biomarkers correlate with stage, demonstrating that the biomarker measurements progress with extent of disease and correlate with tumor size and invasion to the lymph nodes and metastasis. These results support the utility of the biomarkers in recurrence monitoring.

The model provides additional prognostic input prior to surgery, which may guide neoadjuvant or surgical treatment choices. It also provides information for patients who are not surgical candidates (the Unknown Stage category). Stage is not a perfect predictor; there are early stage disease patients with EVD and late stage patients with NED after resection. The Outcome model outperformed Stage alone for prognosis (Table 8). The additional evidence the blood test provides prior to surgery may avoid unnecessary post-surgical chemotherapy in the NED group and strengthen the decision for follow-up systemic therapy in the EVD group.

### Example 6

### Correlation of the Outcome SOMAmers with Recurrence

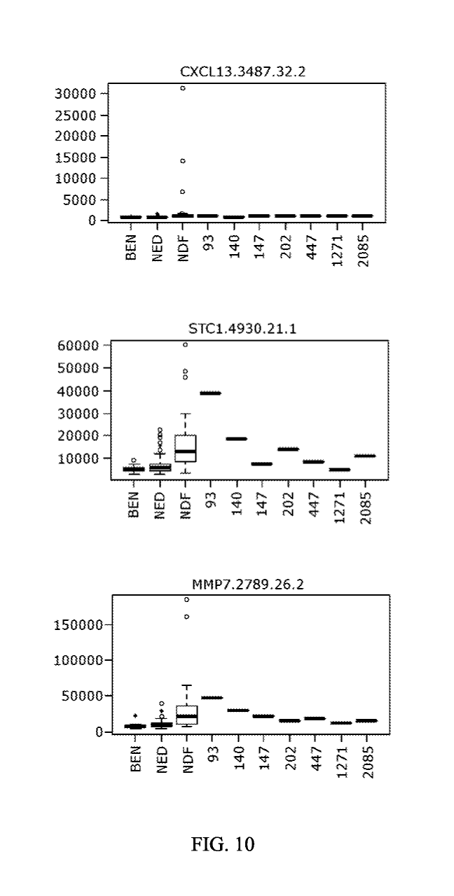

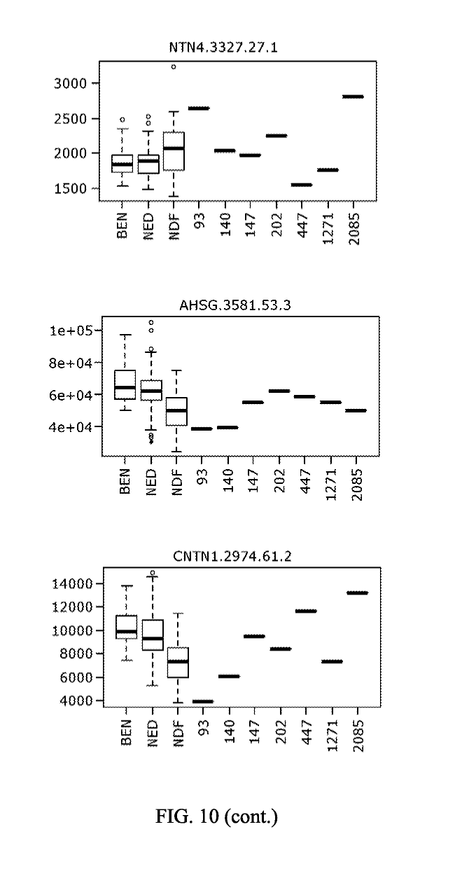

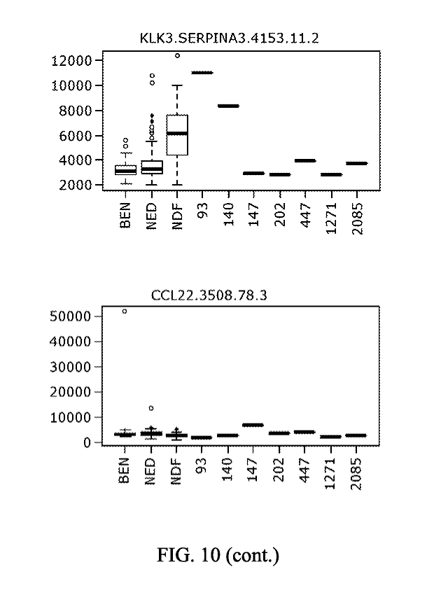

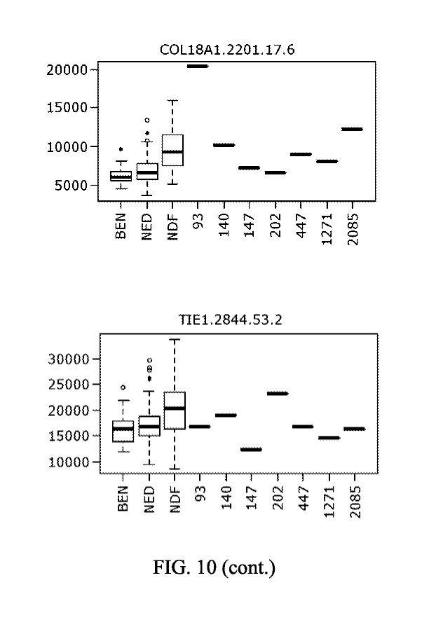

There were only 7 documented recurrences of RCC in this study; the remainder of the EVD case group were never free of disease. The Outcome classifier correctly predicted recurrence in the TP1 samples of four of these subjects, and correct predictions correlated with days from TP1 blood collection to recurrence. Not surprisingly, the differential expression of the biomarkers also correlated with days from TP1 to recurrence (FIG. 10). In particular, up-regulated STC1, MMP7, KLK3.SerpinA3, and COL18A1 and down-regulated AHSG and CNTN1 trend with days to recurrence. These results provide preliminary evidence that these biomarkers will have utility in detecting recurrent RCC. The accuracy of these markers for monitoring recurrence will be strengthened by comparing within a subject change over time during the routine course of post-surgical monitoring. Thus multiple tests may be ordered by the oncologist during SOC (standard of care) follow-up of these patients.

### Example 7

### Classifier Performance on Blinded Verification Set

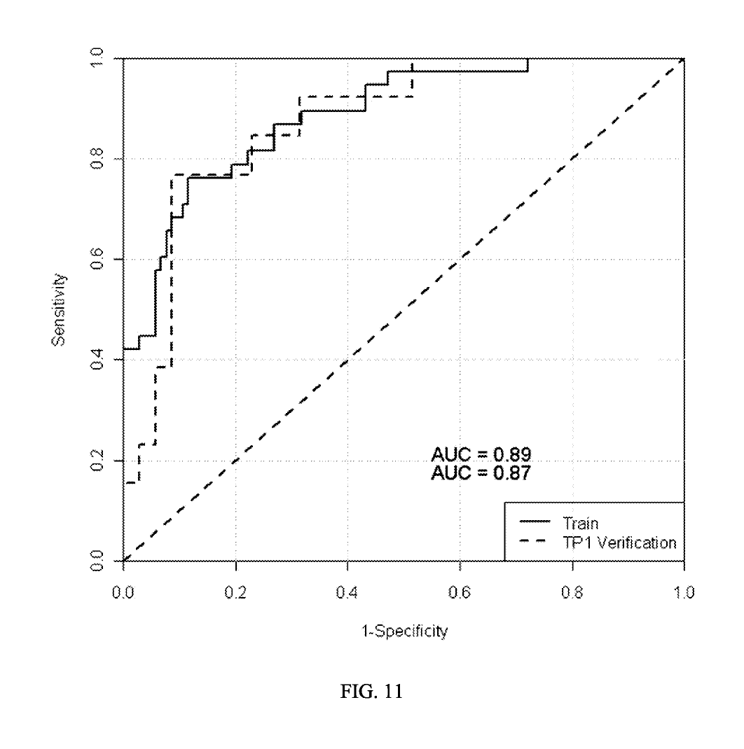

The clinical and assay data for 104 serum samples (25% of total study) were blinded until the Outcome classifier was finalized. Tables 9 and 10 contain a description of this cohort. The demographics are similar to the training set. Two samples were excluded from loss to follow-up. The RF prediction score was generated for these samples and the clinical identity unblinded by a third party independent of SomaLogic. The performance of the TP1 blinded verification set is nearly identical to that of the training set with an ROC of 0.87, verifying the performance of the classifier for pre-surgical or pre-treatment prognosis (FIG. 11). The performance in the smaller TP2 set is consistent, although lower than the TP1 data with an ROC of 0.75

### Example 8

### Diagnosis of RCC

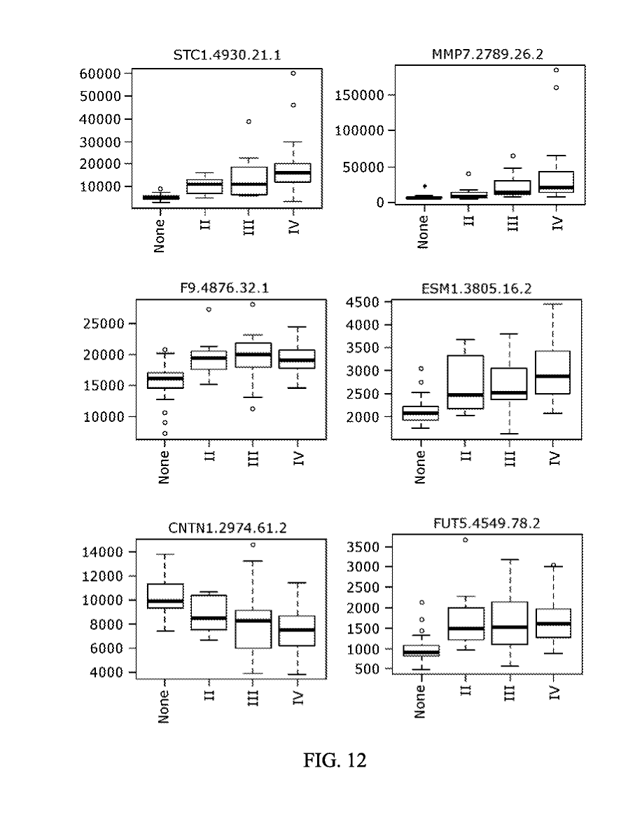

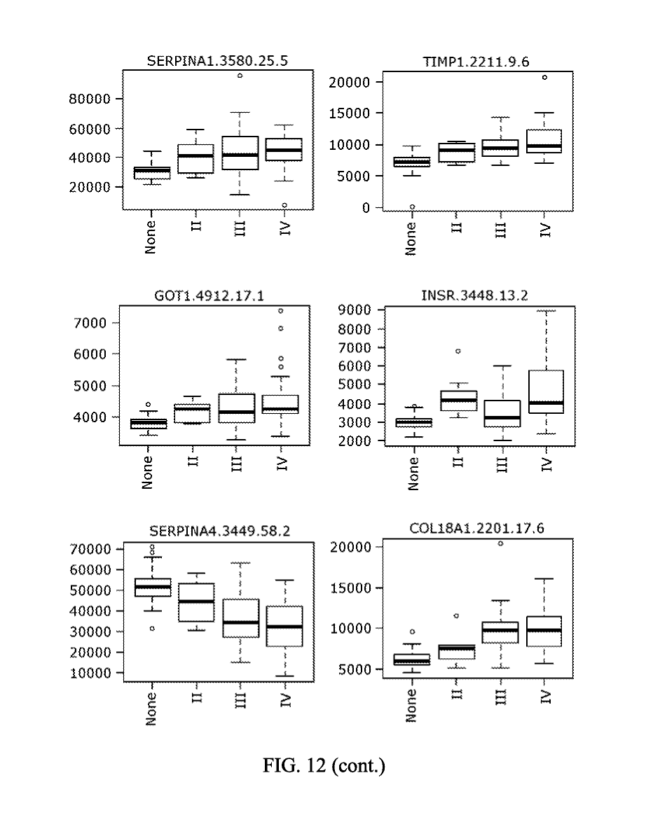

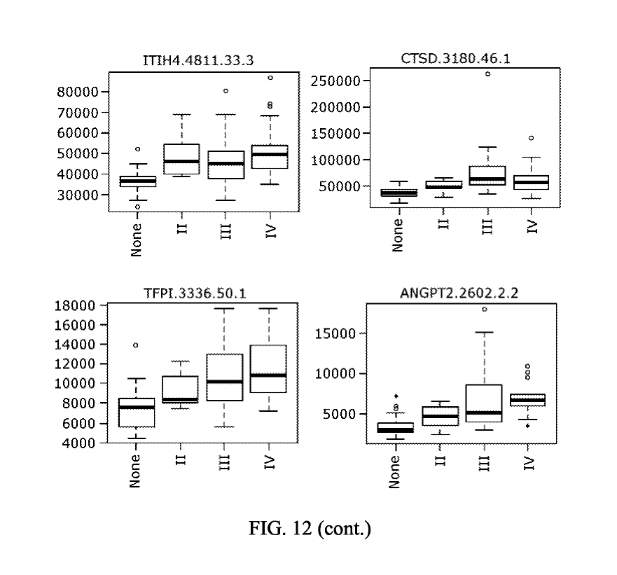

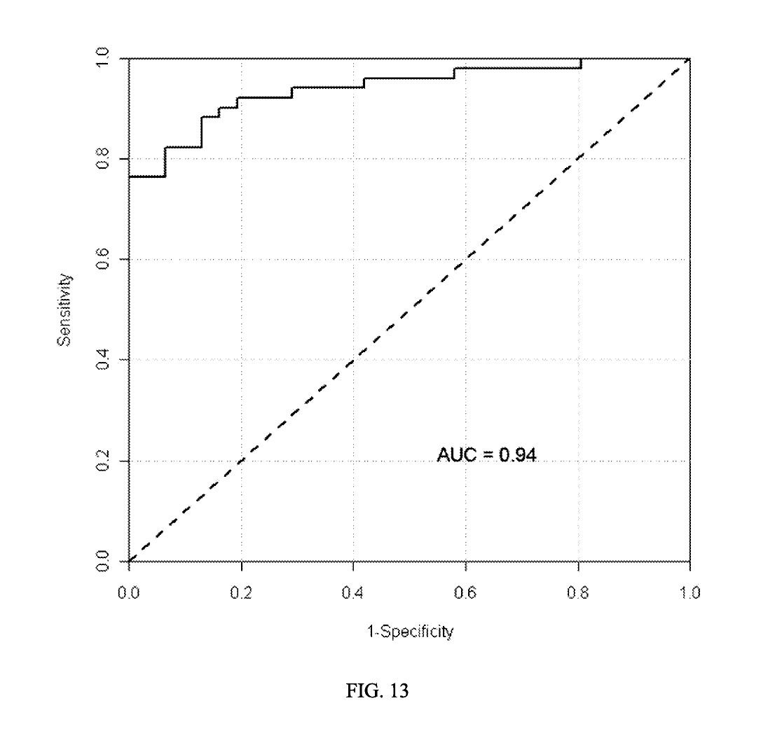

Biomarkers that differentiate benign renal mass (BEN) from Stage II-IV RCC were discovered by comparing the SOMAmer values for pre-surgery TP1 from 31 Benign controls vs. 49 Stage II-IV RCC cases (see Table 4 for RCC stage distribution). A total of 106 markers demonstrated significant differential expression, defined by KS q-value<0.01. These markers were used in backwards selection to develop random forest classifiers. The resulting 16-biomarker classifier and Gini scores are shown in Table 11. As few as 3 markers gives an AUC>0.9. The distribution of the 16 biomarkers by RCC stage is shown in FIG. 12. There is a progression from benign through Stage IV for all markers, whether up or down-regulated. An ROC curve for the 16 biomarkers and random forest classifier is shown in FIG. 13, with an AUC of 0.94 to distinguish benign renal mass from Stages II-IV RCC.

### Example 9

### Discovery of the Disease Burden Vector

The TNM (tumor-node-metastatis) staging system for RCC defines the anatomic extent of disease, and stage has been shown to correlate with prognosis. We discovered biomarkers in the T1 blood sample of RCC patients and benign renal mass controls that correlate with stage, and developed a method to assess the magnitude of the disease burden prior to treatment and surgery. These markers were incorporated into a Disease Burden Vector (DBV) as described below.

A large set of potential biomarkers that correlate with RCC stage were identified with the Jonckheere-Terpstra (JT) trend test for each individual protein, which is a nonparametric test for ordering differences among classes (P. Broberg. Statistical analysis of the genechip. Statistics, 3:1-27, 2005). The samples used for biomarker discovery included benign renal mass and RCC stages I-IV (Tables 2-4). A total of 100 potential biomarkers were discovered at a significance level below 0.01 after Bonferroni correction for multiple comparisons. The biomarkers were selected as DBV model candidates from the JT test results with Sparse Generalized Partial Least Squares (SGPLS) regression (D. Chung, S. Keles, et al. Sparse partial least squares classification for high dimensional data. Statistical applications in genetics and molecular biology, 9(1):17, 2010) or with LASSO using a multinomial model (J. Friedman, T. Hastie, and R. Tibshirani. Regularization paths for generalized linear models via coordinate descent. Journal of statistical software, 33(1):1, 2010). The final DBV model was created using PCA.

A disease burden vector (DBV) is composed of a series of numbers, or coefficients, each of which is associated with a particular protein. A DBV can be applied to a set of protein measurements derived from a patient sample to determine a DBV score in the following manner. Only the proteins measurements from the patient sample that correspond the proteins that compose the DBV are used in the calculation. Each protein measurement is multiplied by the corresponding coefficient in the DBV, which is the coefficient that is associated with the same protein. These products are then added together to create a single DBV score. The following paragraph provides a formal description the calculation of a DBV score.

Let \vec{v} be the disease burden vector, with n coefficients that correspond to n proteins. Let \vec{x} be the vector of n protein measurements from a clinical sample in the same protein order as the n coefficients in \vec{v}. A disease burden score is calculated by performing the dot-product of the disease burden vector and the sample vector as follows: Σᵢ₌₁ⁿvᵢxᵢ, where vᵢ is the i^{th} element in the vector \vec{v}.

Tables 19 and 20, representing different panels, set forth DBV coefficients which can be used to calculate the DBV score. To arrive at the disease burden, the coefficient is multiplied by the measured biomarker value. The disease burdens for each biomarker of the panel are then added to produce the total disease burden for the individual as determined by the panel.

SGPLS Analysis for Biomarker Selection and DBV Model

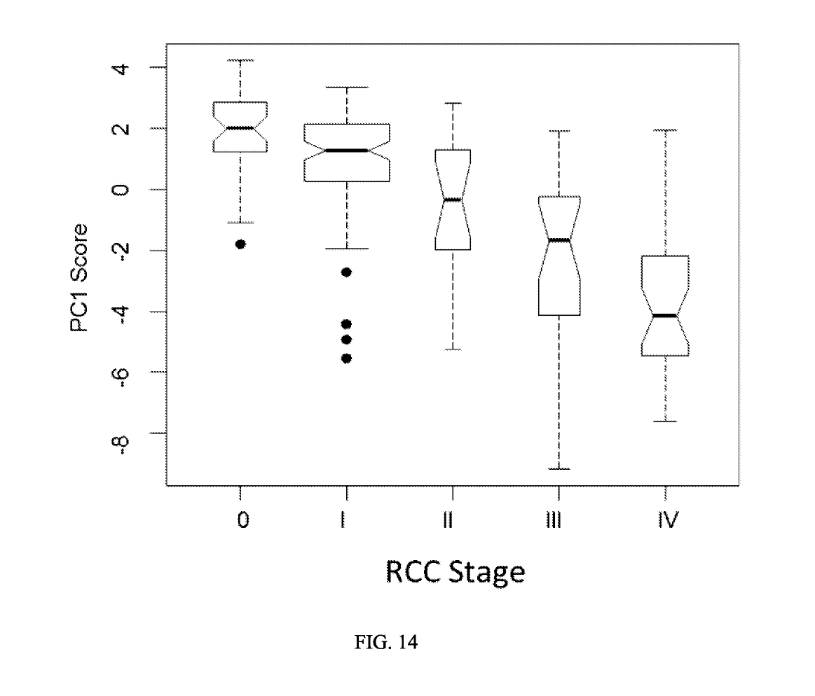

To reduce the number of proteins for modeling the DBV, SGPLS regression was applied to the significant markers identified by the JT test. Ten-fold cross-validation was applied with 10 replicates for identifying the penalty parameter n and the number of SGPLS components K. Table 12 shows the selected proteins. The box plot in FIG. 14 shows the relationship between the pathologic RCC stage and the estimated tumor stage by the DBV model derived from the biomarkers selected by SGPLS. The DBV was modeled using PCA, and the score for Principle Component 1 is plotted as a function of pathologic stage. The DBV score decreases as the extent of RCC increases.

LASSO Multinomial Model for Biomarker Selection and DBV Model

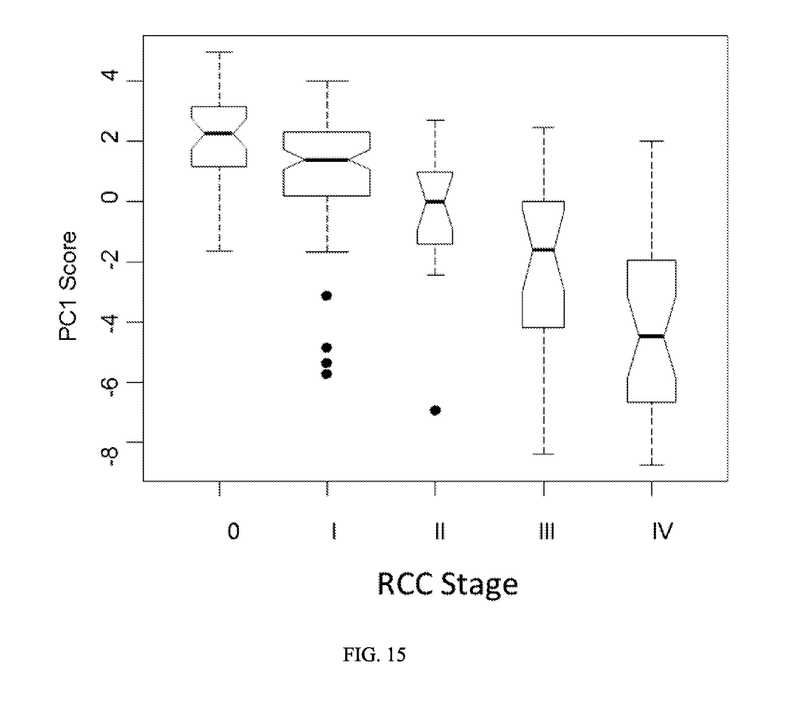

A second multinomial model was constructed using LASSO to select an alternative candidate list of DBV biomarkers. Table 13 lists the LASSO selected proteins and FIG. 15 shows the correlation with pathologic stage of the DBV model constructed with the proteins identified by LASSO. The PC1 score is anti-correlated with RCC stage, where lower numbers indicate a larger disease burden.

### Example 10

### Naive Bayesian Classification for RCC

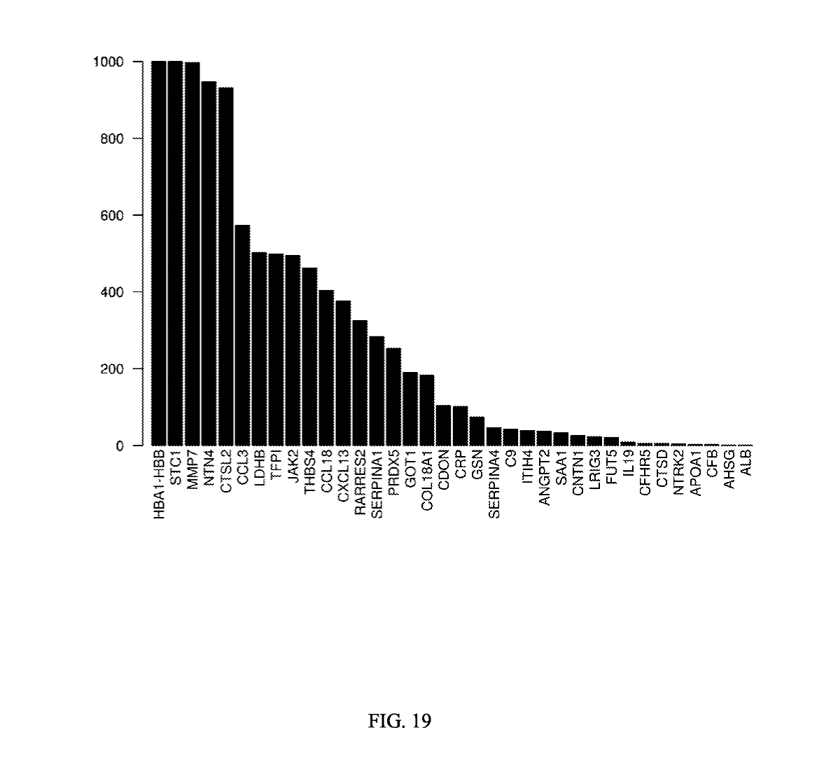

Using the 48 analytes in Table 1, a total of 918 10-analyte classifiers were found with a cross-validation AUC of 0.91 for determining EVD from the control NED group. From this set of classifiers, a total of 13 biomarkers were found to be present in 30% or more of the high scoring classifiers. Table 14 provides a list of these potential biomarkers and FIG. 19 is a frequency plot for the identified biomarkers.

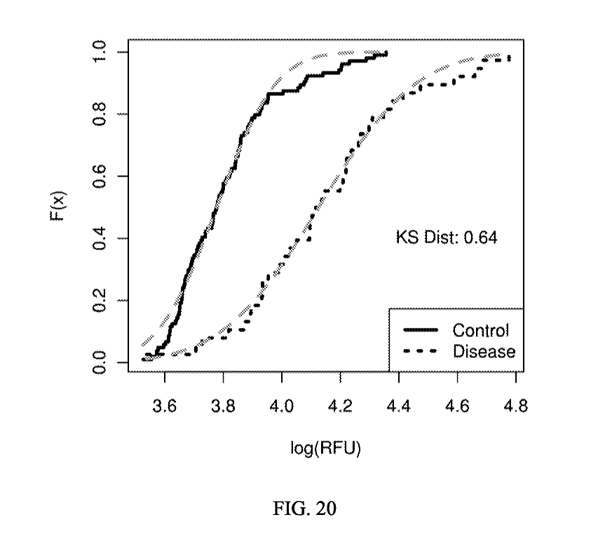

The class-dependent probability density functions (pdfs), p(xᵢ|c) and p(xᵢ|d), where xᵢ is the log of the measured RFU value for biomarker i, and c and d refer to the control and disease populations, were modeled as log-normal distribution functions characterized by a mean μ and variance σ². The parameters for pdfs of the ten biomarkers are listed in Table 15 and an example of the raw data along with the model fit to a normal pdf is displayed in FIG. 20. The underlying assumption appears to fit the data quite well as evidenced by FIG. 20.

The naïve Bayes classification for such a model is given by the following equation, where p(d) is the prevalence of the disease in the population,

$$ln ( \frac{p ( \tilde{d} \vert x )}{p ( \tilde{c} \vert x )} ) = {\sum }_{i = 1}^{n} ln ( \frac{{σ}_{c , i}}{{σ}_{d , i}} ) - \frac{1}{2} {\sum }_{i = 1}^{n} [ ( \frac{{x}_{i} - {μ}_{d , i}^{2}}{{σ}_{d , i}} ) - ( \frac{{x}_{i} - {μ}_{c , i}^{2}}{{σ}_{c , i}} ) + ln ] ( \frac{p ( d )}{1 - p ( d )} )$$

appropriate to the test and n=10. Each of the terms in the summation is a log-likelihood ratio for an individual marker and the total log-likelihood ratio of a sample \tilde{x} being free from the disease of interest (i.e. in this case, NED) versus having the disease (EVD) is simply the sum of these individual terms plus a term that accounts for the prevalence of the disease. For simplicity, we assume p(d)=0.5 so that

$$ln ( \frac{p ( d )}{1 - p ( d )} ) = 0.$$

Given an unknown sample measurement in log(RFU) for each of the ten biomarkers of 8.8, 8.1, 7.6, 9.0, 8.8, 6.1, 6.9, 7.2, 7.4, 8.5, the calculation of the classification is detailed in Table 17. The individual components comprising the log likelihood ratio for disease versus control class are tabulated and can be computed from the parameters in Table 17 and the values of \tilde{x}. The sum of the individual log likelihood ratios is −6.822, or a likelihood of being free from the disease versus having the disease of 918, where likelihood e^{6.822}=918. The first biomarker value has a likelihood more consistent with the disease group (log likelihood>0) but the remaining 9 biomarkers are all consistently found to favor the control group. Multiplying the likelihoods together gives the same results as that shown above; a likelihood of 918 that the unknown sample is free from the disease. In fact, this sample came from the control population in the training set.

### Example 11

### Greedy Algorithm for Selecting Biomarker Panels for Classifiers Part 1

This example describes the selection of biomarkers from Table 1 to form panels that can be used as classifiers in any of the methods described herein. Subsets of the biomarkers in Table 1 were selected to construct classifiers with good performance.

The measure of classifier performance used here is the cross validation AUC; a performance of 0.5 is the baseline expectation for a random (coin toss) classifier, a classifier worse than random would score between 0.0 and 0.5, a classifier with better than random performance would score between 0.5 and 1.0. A perfect classifier with no errors would have a sensitivity of 1.0 and a specificity of 1.0. One can apply the methods described in Example 10 to other common measures of performance such as the F-measure, the sum of sensitivity and specificity, or the product of sensitivity and specificity. Specifically one might want to treat sensitivity and specificity with differing weight, so as to select those classifiers which perform with higher specificity at the expense of some sensitivity, or to select those classifiers which perform with higher sensitivity at the expense of some specificity. Since the method described here only involves a measure of “performance”, any weighting scheme which results in a single performance measure can be used. Different applications will have different benefits for true positive and true negative findings, and also different costs associated with false positive findings from false negative findings. For example, predicting Outcome in RCC patients and detecting RCC recurrence may not in general have the same optimal trade-off between specificity and sensitivity. The different demands of the two tests will in general require setting different weighting to positive and negative misclassifications, reflected in the performance measure. Changing the performance measure will in general change the exact subset of markers selected from Table 1 for a given set of data.

For the Bayesian approach to the determination of Outcome EVD samples from control NED samples described in Example 10, the classifier was completely parameterized by the distributions of biomarkers in the disease (EVD) and control (NED) training samples, and the list of biomarkers was chosen from Table 1; that is to say, the subset of markers chosen for inclusion determined a classifier in a one-to-one manner given a set of training data.

The greedy method employed here was used to search for the optimal subset of markers from Table 1. For small numbers of markers or classifiers with relatively few markers, every possible subset of markers was enumerated and evaluated in terms of the performance of the classifier constructed with that particular set of markers (see Example 11, Part 2). (This approach is well known in the field of statistics as “best subset selection”; see, e.g., Hastie et al). However, for the classifiers described herein, the number of combinations of multiple markers can be very large, and it was not feasible to evaluate every possible set of 10 markers, as there are 30,045,015 possible combinations that can be generated from a list of only 30 total analytes. Because of the impracticality of searching through every subset of markers, the single optimal subset may not be found; however, by using this approach, many excellent subsets were found, and, in many cases, any of these subsets may represent an optimal one.

Instead of evaluating every possible set of markers, a “greedy” forward stepwise approach may be followed (see, e.g., Dabney A R, Storey J D (2007) Optimality Driven Nearest Centroid Classification from Genomic Data.

PLoS ONE 2(10): e1002. doi:10.1371/journal.pone.0001002). Using this method, a classifier is started with the best single marker (based on KS-distance for the individual markers) and is grown at each step by trying, in turn, each member of a marker list that is not currently a member of the set of markers in the classifier. The one marker which scores best in combination with the existing classifier is added to the classifier. This is repeated until no further improvement in performance is achieved. Unfortunately, this approach may miss valuable combinations of markers for which some of the individual markers are not all chosen before the process stops.

The greedy procedure used here was an elaboration of the preceding forward stepwise approach, in that, to broaden the search, rather than keeping just a single candidate classifier (marker subset) at each step, a list of candidate classifiers was kept. The list was seeded with every single marker subset (using every marker in the table on its own). The list was expanded in steps by deriving new classifiers (marker subsets) from the ones currently on the list and adding them to the list. Each marker subset currently on the list was extended by adding any marker from Table 1 not already part of that classifier, and which would not, on its addition to the subset, duplicate an existing subset (these are termed “permissible markers”). Every existing marker subset was extended by every permissible marker from the list. Clearly, such a process would eventually generate every possible subset, and the list would run out of space. Therefore, all the generated classifiers were kept only while the list was less than some predetermined size (often enough to hold all three marker subsets). Once the list reached the predetermined size limit, it became elitist; that is, only those classifiers which showed a certain level of performance were kept on the list, and the others fell off the end of the list and were lost. This was achieved by keeping the list sorted in order of classifier performance; new classifiers which were at least as good as the worst classifier currently on the list were inserted, forcing the expulsion of the current bottom underachiever. One further implementation detail is that the list was completely replaced on each generational step; therefore, every classifier on the list had the same number of markers, and at each step the number of markers per classifier grew by one.

Since this method produced a list of candidate classifiers using different combinations of markers, one may ask if the classifiers can be combined in order to avoid errors which might be made by the best single classifier, or by minority groups of the best classifiers. Such “ensemble” and “committee of experts” methods are well known in the fields of statistical and machine learning and include, for example, “Averaging”, “Voting”, “Stacking”, “Bagging” and “Boosting” (see, e.g., Hastie et al.). These combinations of simple classifiers provide a method for reducing the variance in the classifications due to noise in any particular set of markers by including several different classifiers and therefore information from a larger set of the markers from the biomarker table, effectively averaging between the classifiers. An example of the usefulness of this approach is that it can prevent outliers in a single marker from adversely affecting the classification of a single sample. The requirement to measure a larger number of signals may be impractical in conventional “one marker at a time” antibody assays but has no downside for a fully multiplexed aptamer assay. Techniques such as these benefit from a more extensive table of biomarkers and use the multiple sources of information concerning the disease processes to provide a more robust classification.

The biomarkers selected in Table 1 gave rise to classifiers which perform better than classifiers built with “non-markers” (i.e., proteins having signals that did not meet the criteria for inclusion in Table 1).

For classifiers containing only one, two, and three markers, all possible classifiers obtained using the biomarkers in Table 1 were enumerated and examined for the distribution of performance compared to classifiers built from a similar table of randomly selected non-markers signals.

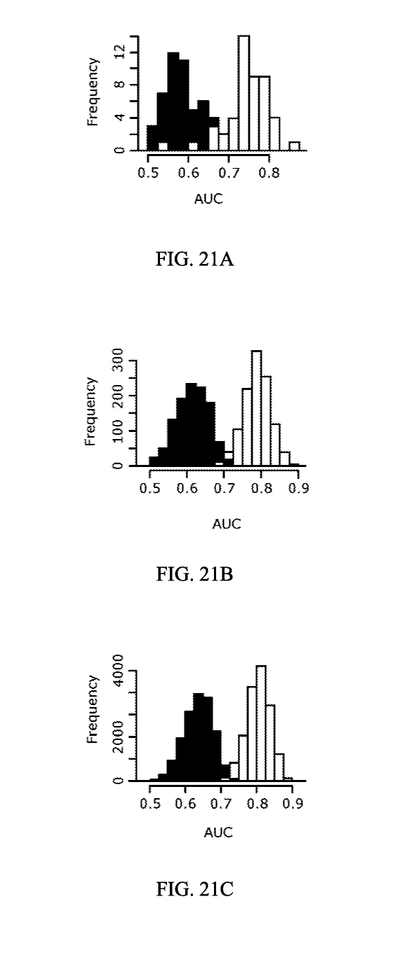

In FIG. 21, the AUC was used as the measure of performance; a performance of 0.5 is the baseline expectation for a random (coin toss) classifier. The histogram of classifier performance was compared with the histogram of performance from a similar exhaustive enumeration of classifiers built from a “non-marker” table of 48 non-marker signals; the 48 signals were randomly chosen from aptamers that did not demonstrate differential signaling between control and disease populations.

FIG. 21 shows histograms of the performance of all possible one, two, and three-marker classifiers built from the biomarker parameters in Table 15 for biomarkers that can discriminate between the control NED group and disease EVD group and compares these classifiers with all possible one, two, and three-marker classifiers built using the 48 “non-marker” aptamer RFU signals. FIG. 21A shows the histograms of single marker classifier performance, FIG. 21B shows the histogram of two marker classifier performance, and FIG. 21C shows the histogram of three marker classifier performance.

In FIG. 21, the empty bars represent the histograms of the classifier performance of all one, two, and three-marker classifiers using the biomarker data for NED and EVD groups in Tables 2-5. The black bars are the histograms of the classifier performance of all one, two, and three-marker classifiers using the data for NED and EVD but using the set of random non-marker signals.

The classifiers built from the markers listed in Table 1 form a distinct histogram, well separated from the classifiers built with signals from the “non-markers” for all one-marker, two-marker, and three-marker comparisons. The performance and AUC score of the classifiers built from the biomarkers in Table 1 also increase faster with the number of markers than do the classifiers built from the non-markers, the separation increases between the marker and non-marker classifiers as the number of markers per classifier increases. All classifiers built using the biomarkers listed in Table 1 perform distinctly better than classifiers built using the “non-markers”.

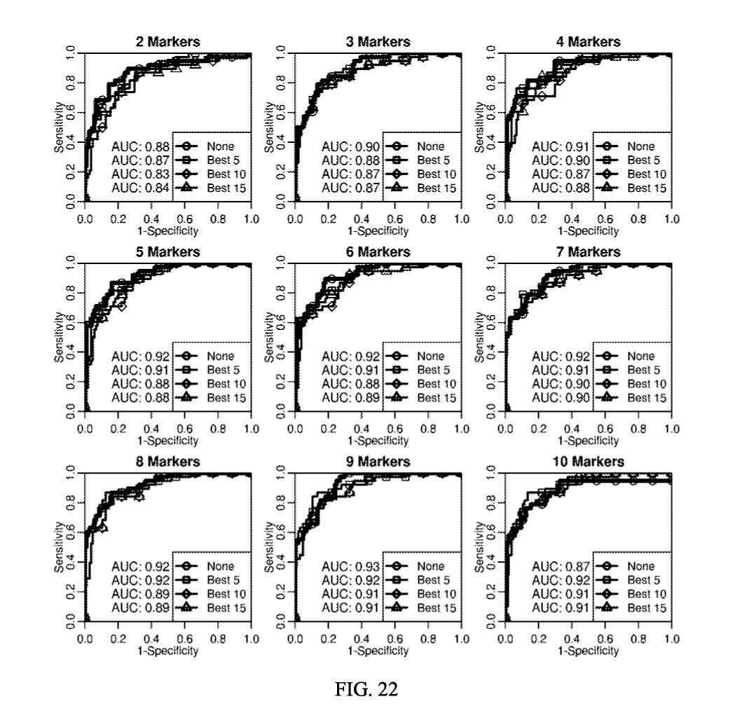

The distributions of classifier performance show that there are many possible multiple-marker classifiers that can be derived from the set of analytes in Table 1. Although some biomarkers are better than others on their own, as evidenced by the distribution of classifier scores and AUCs for single analytes, it was desirable to determine whether such biomarkers are required to construct high performing classifiers. To make this determination, the behavior of classifier performance was examined by leaving out some number of the best biomarkers. FIG. 22 compares the performance of classifiers built with the full list of biomarkers in Table 1 with the performance of classifiers built with subsets of biomarkers from Table 1 that excluded top-ranked markers.

FIG. 22 demonstrates that classifiers constructed without the best markers perform well, implying that the performance of the classifiers was not due to some small core group of markers and that the changes in the underlying processes associated with disease are reflected in the activities of many proteins. Many subsets of the biomarkers in Table 1 performed close to optimally, even after removing the top 15 of the 48 markers from Table 1. After dropping the 15 top-ranked markers (ranked by KS-distance) from Table 1, the classifier performance increased with the number of markers selected from the table to reach an AUC of almost 0.90, close to the performance of the optimal classifier score of 0.875 selected from the full list of biomarkers.

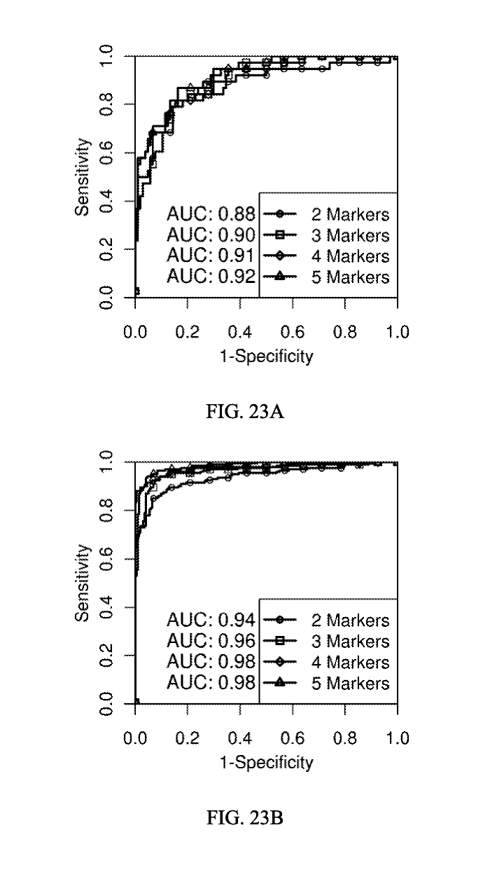

Finally, FIG. 23 shows how the ROC performance of typical classifiers constructed from the list of parameters in Table 15 according to Example 10. A five analyte classifier was constructed with STC1, CXCL13, MMP7, RARRES2, and HBA1-HBB. FIG. 23A shows the performance of the model, assuming independence of these markers, as in Example 10, and FIG. 23B shows the empirical ROC curves generated from the study data set used to define the parameters in Table 15. It can be seen that the performance for a given number of selected markers was qualitatively in agreement, and that quantitative agreement was generally quite good, as evidenced by the AUCs, although the model calculation tends to overestimate classifier performance. This is consistent with the notion that the information contributed by any particular biomarker concerning the disease processes is redundant with the information contributed by other biomarkers provided in Table 1 while the model calculation assumes complete independence. FIG. 23 thus demonstrates that Table 1 in combination with the methods described in Example 10 enable the construction and evaluation of a great many classifiers useful for the discrimination of EVD from the NED group. Table 18 summarizes the range of performance of the top 1000 classifiers for model sizes 1-10 generated by the greedy algorithm described in Example 11. The maximum AUC ranges from 0.86 for one marker to 0.92 for ten markers.

TABLE 1
Cancer biomarkers

| Column #1 | Column #2 | Column #3 | Column #4 | Column #5 | Column #6 |
|---|---|---|---|---|---|
| | Biomarker Designation | | | | |
| Biomarker # | Entrez Gene Symbol(s) | Entrez Gene ID | SwissProt ID | Public Name | Direction |
| 1 | AFM | 173 | P43652 | Afamin | Down |
| 2 | AHSG | 197 | P02765 | α2-HS-Glycoprotein | Down |
| 3 | ALB | 213 | P02768 | Albumin | Down |
| 4 | ANGPT2 | 285 | O15123 | Angiopoietin-2 | Up |
| 5 | APOA1 | 335 | P02647 | Apolipoprotein A-I | Down |
| 6 | APOE | 348 | P02649 | Apolipoprotein E3 | Down |
| 7 | C9 | 735 | P02748 | Complement C9 | Up |
| 8 | CCL18 | 6362 | P55774 | Macrophage | Up |
| | | | | inflammatory protein | |
| | | | | 4/Pulmonary and | |
| | | | | activation-regulated | |
| | | | | chemokine/CCL18 | |
| 9 | CCL23 | 6368 | P55773 | Myeloid progenitor | Up |
| | | | | inhibitory factor | |
| | | | | 1/CCL23 | |
| 10 | CCL3 | 6348 | P10147 | Macrophage | Down |
| | | | | inflammatory protein 1- | |
| | | | | α/CCL3 | |
| 11 | CDON | 50937 | Q4KMG0 | Cell adhesion molecule- | Down |
| | | | | related down-regulated | |
| | | | | by oncogenes | |
| 12 | CFB | 629 | P00751 | Complement factor B | Up |
| 13 | CFHR5 | 81494 | Q9BXR6 | Complement factor H- | Up |
| | | | | related 5 | |
| 14 | CNTN1 | 1272 | Q12860 | Contactin-1 | Down |
| 15 | CNTN5 | 53942 | O94779 | Contactin-5 | Down |
| 16 | COL18A1 | 80781 | P39060 | Endostatin | Up |
| 17 | CRP | 1401 | P02741 | C-reactive protein | Up |
| 18 | CTSD | 1509 | P07339 | Cathepsin D | Up |
| 19 | CTSL2 | 1515 | O60911 | Cathepsin V | Down |
| 20 | CXCL13 | 10563 | O43927 | B lymphocyte | Up |
| | | | | chemoattractant/CXCL1 | |
| | | | | 3 | |
| 21 | ESM1 | 11082 | Q9NQ30 | Endocan | Up |
| 22 | FUT5 | 2527 | Q11128 | Fucosyltransferase 5 | Up |
| 23 | GOT1 | 2805 | P17174 | Aspartate | Up |
| | | | | aminotransferase | |
| 24 | GSN | 2934 | P06396 | Gelsolin | Down |
| 25 | HBA1-HBB | 3039; ; 3043 | P69905, P68871 | Hemoglobin | Down |
| 26 | IL19 | 29949 | Q9UHD0 | Interleukin-19 | Down |
| 27 | ITIH4 | 3700 | Q14624 | Inter-α-trypsin inhibitor | Up |
| | | | | heavy chain H4 | |
| 28 | JAK2 | 3717 | O60674 | Janus kinase 2 | Up |
| 29 | KLK3-SERPINA3 | 354; 12 | P07288, P01011 | PSA: α-1- | Up |
| | | | | antichymotrypsin | |
| | | | | complex | |
| 30 | LBP | 3929 | P18428 | Lipopolysaccharide- | Up |
| | | | | binding protein | |
| 31 | LDHB | 3945 | P07195 | Lactate dehydrogenase 1 | Up |
| | | | | (heart) | |
| 32 | LRIG3 | 121227 | Q6UXM1 | Leucine-rich repeats and | Down |
| | | | | Ig-like domains protein 3 | |
| 33 | MMP7 | 4316 | P09237 | Matrix | Up |
| | | | | metalloproteinase | |
| | | | | 7/Matrilysin | |
| 34 | NTN4 | 59277 | Q9HB63 | Netrin-4 | Up |
| 35 | NTRK2 | 4915 | Q16620 | Neurotrophic tyrosine | Down |
| | | | | kinase receptor type 2 | |
| 36 | PLA2G2A | 5320 | P14555 | Phospholipase A2, | Up |
| | | | | Group IIA | |
| 37 | PRDX5 | 25824 | P30044 | Peroxiredoxin-5 | Up |
| 38 | RARRES2 | 5919 | Q99969 | Chemerin | Up |
| 39 | SAA1 | 6288 | P02735 | Serum amyloid A | Up |
| 40 | SERPINA1 | 5265 | P01009 | α1-Antitrypsin | Up |
| 41 | SERPINA4 | 5267 | P29622 | Kallistatin | Down |
| 42 | STC1 | 6781 | P52823 | Stanniocalcin-1 | Up |
| 43 | TFPI | 7035 | P10646 | Tissue factor pathway | Up |
| | | | | inhibitor | |
| 44 | TG | 7038 | P01266 | Thyroglobulin | Down |
| 45 | THBS4 | 7060 | P35443 | Thrombospondin-4 | Down |
| 46 | TIMP1 | 7076 | P01033 | Tissue inhibitor of | Up |
| | | | | metalloproteinases 1 | |
| 47 | TNFRSF1A | 7132 | P19438 | Tumor necrosis factor | Up |
| | | | | receptor superfamily | |
| | | | | member 1A | |
| 48 | VEGFA | 7422 | P15692 | Vascular endothelial | Up |
| | | | | growth factor A | |

TABLE 2
Diagnosis for 281 training samples

| Diagnosis | Pre-surgery | Post-surgery |
|---|---|---|
| BEN | 31 | 14 |
| RCC | 142 | 94 |
| TOTAL | 173 | 108 |

TABLE 3
Demographics by Outcome category

| | BEN | NED | EVD |
|---|---|---|---|
| Gender | | | |
| Male | 20 (65%) | 55 (53%) | 22 (58%) |
| Female | 11 (35%) | 49 (47%) | 16 (42%) |
| Age | | | |
| Median | 57 | 60 | 61 |
| Range | 25-80 | 30-90 | 46-81 |

TABLE 4
Outcome of the RCC TP1 training cases by AJCC pTNM

| Stage | NED | EVD |
|---|---|---|
| I | 77 | 2 |
| II | 7 | 1 |
| III | 12 | 5 |
| IV | 1 | 23 |
| None* | 7 | 7 |
| TOTAL | 104 | 38 |

*None are subjects who did not undergo surgery but were diagnosed with RCC clinically

TABLE 5
Subset of subjects with post-surgical TP2 sample

| Stage | NED | EVD |
|---|---|---|
| I | 57 | 1 |
| II | 6 | 0 |
| III | 6 | 2 |
| IV | 0 | 14 |
| None | 7 | 1 |
| TOTAL | 76 | 18 |

TABLE 6
The 16 biomarkers chosen by backwards selection based
on KS or PCA biomarker candidates along with the RF
Gini importance score for each model.

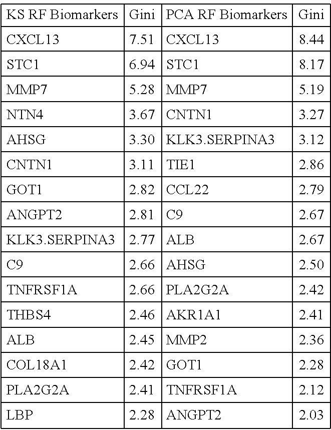

Markers in common are shaded.

TABLE 7
The 10 biomarkers in the TP1 Outcome model and Gini importance scores

| Outcome Model Biomarkers | Gini |
|---|---|
| CXCL13 | 9.83 |
| STC1 | 9.55 |
| MMP7 | 6.79 |
| KLK3.SERPINA3 | 4.91 |
| CNTN1 | 4.36 |
| NTN4 | 4.17 |
| AHSG | 4.00 |
| CCL22 | 3.99 |
| COL18A1 | 3.91 |
| TIE1 | 3.78 |

TABLE 8
Prognosis Accuracy of Outcome Model vs Pathologic Stage

| | NED | EVD |
|---|---|---|
| Outcome Model | 121/135 = 90% | 29/38 = 76% |
| Pathologic Stage | 115/135 = 85% | 28/38 = 74% |

TABLE 9
Blinded verification set cohort by timepoint

| | BEN | RCC NED | RCC EVD |
|---|---|---|---|
| TP1 | 9 | 35 | 13 |
| TP2 | 8 | 28 | 9 |

TABLE 10
Blinded verification set cohort by Outcome and Stage

| Diagnosis | NED | EVD |
|---|---|---|
| BEN | 9 | 0 |
| Stage I | 20 | 2 |
| Stage II | 8 | 1 |
| Stage III | 3 | 3 |
| Stage IV | 1 | 4 |
| Unknown | 3 | 3 |
| TOTAL | 44 | 13 |

TABLE 11
The 16 Biomarkers in the Benign Renal Mass vs Stage II-IV
RCC Classifier and Gini importance scores

| Biomarker | Gini |
|---|---|
| STC1 | 6.81 |
| MMP7 | 4.27 |
| F9 | 2.45 |
| ESM1 | 2.40 |
| CNTN1 | 2.16 |
| FUT5 | 2.07 |
| SERPINA1 | 1.91 |
| TIMP1 | 1.91 |
| GOT1 | 1.90 |
| INSR | 1.90 |
| SERPINA4 | 1.78 |
| COL18A1 | 1.78 |
| ITIH4 | 1.74 |
| CTSD | 1.67 |
| TFPI | 1.64 |
| ANGTP2 | 1.57 |

TABLE 12
DBV Proteins selected with SGPLS

| Index | GeneName | Target |
|---|---|---|
| 1 | STC1 | Stanniocalcin-1 |
| 2 | MMP7 | MMP-7 |
| 3 | SERPINA4 | Kallistatin |
| 4 | COL18A1 | Endostatin |
| 5 | LBP | LBP |
| 6 | GSN | Gelsolin |
| 7 | F9 | Coagulation Factor |
| | | IX |
| 8 | RARRES2 | TIG2 |
| 9 | SLPI | SLPI |
| 10 | F9 | Coagulation Factor |
| | | IX |
| 11 | CCL18 | PARC |
| 12 | INSR | IR |
| 13 | SELL | sL-Selectin |
| 14 | PRDX5 | Peroxi-redoxin-5 |
| 15 | CTSD | Cathepsin D |
| 16 | CTSL2 | Cathepsin V |
| 17 | APOA1 | Apo A-I |
| 18 | CXCL13 | BLC |

TABLE 13
DBV Proteins Selected with LASSO

| Index | GeneName | Target |
|---|---|---|
| 1 | STC1 | Stanniocalcin-1 |
| 2 | MMP7 | MMP-7 |
| 3 | COL18A1 | Endostatin |
| 4 | HP | Haptoglobin, Mixed Type |
| 5 | CNTN1 | contactin-1 |
| 6 | GSN | Gelsolin |
| 7 | ESM1 | Endocan |
| 8 | VEGFA | VEGF |
| 9 | F9 | Coagulation Factor IX |
| 10 | SLPI | SLPI |
| 11 | SAA1 | SAA |
| 12 | CFI | Factor I |
| 13 | CCL18 | PARC |
| 14 | LDHB | LDH-H 1 |
| 15 | GOT1 | GOT1 |
| 16 | CDON | CDON |
| 17 | INSR | IR |
| 18 | SELL | sL-Selectin |
| 19 | HAMP | LEAP-1 |
| 20 | NTRK2 | TrkB |
| 21 | PRDX5 | Peroxiredoxin-5 |
| 22 | CTSD | Cathepsin D |
| 23 | IL19 | IL-19 |
| 24 | CCL3 | MIP-1a |
| 25 | IL1R1 | IL-1 sRI |
| 26 | CXCL13 | BLC |
| 27 | TG | Thyroglobulin |

TABLE 14
Highest frequency 13 analytes in all ten marker naive Bayes classifiers

| | |
|---|---|
| HBA1-HBB | STC1 |
| MMP7 | NTN4 |
| CTSL2 | CCL3 |
| LDHB | JAK2 |
| TFPI | THBS4 |
| CCL18 | CXCL13 |
| RARRES2 | |

TABLE 15
Parameters derived from training set for naïve Bayes classifier.

| Biomarker | μ_{c} | μ_{d} | σ_{c} | σ_{d} |
|---|---|---|---|---|
| COL18A1 | 8.791 | 9.127 | 0.223 | 0.305 |
| CFHR5 | 9.089 | 9.475 | 0.251 | 0.362 |
| IL19 | 10.950 | 10.836 | 0.244 | 0.186 |
| SERPINA1 | 10.331 | 10.690 | 0.227 | 0.395 |
| CCL23 | 8.348 | 8.786 | 0.253 | 0.532 |
| FUT5 | 6.930 | 7.390 | 0.286 | 0.420 |
| ANGPT2 | 8.191 | 8.780 | 0.343 | 0.393 |
| SERPINA4 | 10.781 | 10.380 | 0.182 | 0.472 |
| CRP | 8.317 | 10.592 | 1.630 | 1.385 |
| TFPI | 9.017 | 9.289 | 0.205 | 0.345 |
| PRDX5 | 7.681 | 7.807 | 0.226 | 0.235 |
| PLA2G2A | 9.513 | 10.495 | 0.477 | 1.289 |
| CNTN5 | 6.583 | 6.502 | 0.096 | 0.061 |
| CNTN1 | 9.136 | 8.904 | 0.192 | 0.261 |
| C9 | 11.783 | 12.104 | 0.230 | 0.280 |
| STC1 | 8.698 | 9.501 | 0.367 | 0.600 |
| JAK2 | 9.024 | 9.267 | 0.165 | 0.190 |
| APOA1 | 8.562 | 8.413 | 0.169 | 0.250 |
| CDON | 10.288 | 10.043 | 0.223 | 0.285 |
| ITIH4 | 10.564 | 10.821 | 0.149 | 0.203 |
| TNFRSF1A | 8.113 | 8.377 | 0.144 | 0.220 |
| HBA1-HBB | 7.463 | 6.947 | 0.532 | 0.457 |
| CCL18 | 10.185 | 10.624 | 0.428 | 0.451 |
| TG | 6.121 | 6.098 | 0.049 | 0.067 |
| VEGFA | 7.603 | 7.775 | 0.138 | 0.227 |
| CCL3 | 6.146 | 6.241 | 0.177 | 0.170 |
| TIMP1 | 8.947 | 9.189 | 0.150 | 0.264 |
| GOT1 | 8.259 | 8.372 | 0.086 | 0.175 |
| ALB | 9.605 | 9.372 | 0.129 | 0.313 |
| THBS4 | 8.821 | 8.609 | 0.219 | 0.287 |
| MMP7 | 9.099 | 9.925 | 0.387 | 0.785 |
| LBP | 8.351 | 9.060 | 0.418 | 0.600 |
| LDHB | 7.208 | 7.158 | 0.182 | 0.310 |
| NTRK2 | 7.046 | 6.959 | 0.128 | 0.168 |
| GSN | 7.524 | 7.287 | 0.185 | 0.287 |
| CTSL2 | 6.152 | 6.100 | 0.072 | 0.065 |
| CTSD | 10.775 | 10.971 | 0.325 | 0.400 |
| ESM1 | 7.702 | 7.887 | 0.184 | 0.289 |
| RARRES2 | 8.041 | 8.226 | 0.227 | 0.201 |
| LRIG3 | 7.301 | 7.207 | 0.081 | 0.111 |
| NTN4 | 7.542 | 7.636 | 0.100 | 0.202 |
| AFM | 10.362 | 10.002 | 0.140 | 0.475 |
| SAA1 | 7.663 | 9.450 | 1.128 | 1.670 |
| CXCL13 | 6.897 | 7.018 | 0.055 | 0.187 |
| CFB | 10.357 | 10.546 | 0.173 | 0.137 |
| AHSG | 11.035 | 10.821 | 0.151 | 0.260 |
| KLK3-SERPINA3 | 8.088 | 8.683 | 0.234 | 0.481 |
| APOE | 10.956 | 10.762 | 0.236 | 0.206 |

TABLE 16
AUC for exemplary combinations of biomarkers

| # | | | | | | | | | | | AUC |
|---|---|---|---|---|---|---|---|---|---|---|---|
| 1 | STC1 | | | | | | | | | | 0.862 |
| 2 | STC1 | CXCL13 | | | | | | | | | 0.825 |
| 3 | STC1 | CXCL13 | MMP7 | | | | | | | | 0.833 |
| 4 | STC1 | CXCL13 | MMP7 | RARRES2 | | | | | | | 0.836 |
| 5 | STC1 | CXCL13 | MMP7 | RARRES2 | HBA1-HBB | | | | | | 0.843 |
| 6 | STC1 | CXCL13 | MMP7 | RARRES2 | HBA1-HBB | THBS4 | | | | | 0.857 |
| 7 | STC1 | CXCL13 | MMP7 | RARRES2 | HBA1-HBB | THBS4 | TFPI | | | | 0.859 |
| 8 | STC1 | CXCL13 | MMP7 | RARRES2 | HBA1-HBB | THBS4 | TFPI | NTN4 | | | 0.867 |
| 9 | STC1 | CXCL13 | MMP7 | RARRES2 | HBA1-HBB | THBS4 | TFPI | NTN4 | CTSL2 | | 0.872 |
| 10 | STC1 | CXCL13 | MMP7 | RARRES2 | HBA1-HBB | THBS4 | TFPI | NTN4 | CTSL2 | LDHB | 0.875 |

TABLE 17
Calculations derived from training set for naïve Bayes classifier.

| | | | | | | | | ln(p(\tilde{d}\|x)/ |
|---|---|---|---|---|---|---|---|---|
| Biomarker | μ_{c} | μ_{d} | σ_{c} | σ_{d} | \tilde{ }x | p(\tilde{c}\|x) | p(\tilde{d}\|x) | p(\tilde{c}\|x)) |
| LDHB | 7.208 | 7.158 | 0.182 | 0.310 | 7.190 | 2.175 | 1.282 | −0.529 |
| TFPI | 9.017 | 9.289 | 0.205 | 0.345 | 9.028 | 1.941 | 0.869 | −0.804 |
| STC1 | 8.698 | 9.501 | 0.367 | 0.600 | 8.489 | 0.925 | 0.161 | −1.750 |
| RARRES2 | 8.041 | 8.226 | 0.227 | 0.201 | 8.082 | 1.728 | 1.536 | −0.118 |
| HBA1- | 7.463 | 6.947 | 0.532 | 0.457 | 7.394 | 0.744 | 0.542 | −0.318 |
| HBB | | | | | | | | |
| THBS4 | 8.821 | 8.609 | 0.219 | 0.287 | 8.808 | 1.815 | 1.093 | −0.507 |
| MMP7 | 9.099 | 9.925 | 0.387 | 0.785 | 8.796 | 0.758 | 0.180 | −1.435 |
| CXCL13 | 6.897 | 7.018 | 0.055 | 0.187 | 6.891 | 7.219 | 1.690 | −1.452 |
| NTN4 | 7.542 | 7.636 | 0.100 | 0.202 | 7.588 | 3.596 | 1.916 | −0.630 |
| CTSL2 | 6.152 | 6.100 | 0.072 | 0.065 | 6.061 | 2.497 | 5.127 | 0.720 |

TABLE 18
Greedy Algorithm Cross Validation AUC Summary Table

| Model Size | Min. | 1st Quartile | Median | Mean | 3rd Quartile | Max. |
|---|---|---|---|---|---|---|
| 1 | 0.50 | 0.71 | 0.73 | 0.72 | 0.76 | 0.86 |
| 2 | 0.72 | 0.75 | 0.78 | 0.78 | 0.80 | 0.87 |
| 3 | 0.84 | 0.85 | 0.85 | 0.85 | 0.86 | 0.89 |
| 4 | 0.87 | 0.87 | 0.88 | 0.88 | 0.88 | 0.90 |
| 5 | 0.89 | 0.89 | 0.89 | 0.89 | 0.89 | 0.92 |
| 6 | 0.90 | 0.90 | 0.90 | 0.90 | 0.90 | 0.92 |
| 7 | 0.90 | 0.90 | 0.90 | 0.91 | 0.91 | 0.92 |
| 8 | 0.91 | 0.91 | 0.91 | 0.91 | 0.91 | 0.92 |
| 9 | 0.91 | 0.91 | 0.91 | 0.91 | 0.91 | 0.92 |
| 10 | 0.91 | 0.91 | 0.91 | 0.91 | 0.91 | 0.92 |

TABLE 19
First Panel of DBV Coefficients

| Index | GeneName | Target | DBV Coefficient |
|---|---|---|---|
| 1 | STC1 | Stanniocalcin-1 | −0.3062 |
| 2 | MMP7 | MMP-7 | −0.2545 |
| 3 | SERPINA4 | Kallistatin | 0.2928 |
| 4 | COL18A1 | Endostatin | −0.2867 |
| 5 | LBP | LBP | −0.2924 |
| 6 | GSN | Gelsolin | 0.2607 |
| 7 | F9 | Coagulation Factor IX | −0.2127 |
| 8 | RARRES2 | TIG2 | −0.2871 |
| 9 | SLPI | SLPI | -0.2524 |
| 10 | F9 | Coagulation Factor IX | −0.1989 |
| 11 | CCL18 | PARC | −0.2498 |
| 12 | INSR | IR | −0.1586 |
| 13 | SELL | sL-Selectin | 0.2083 |
| 14 | PRDX5 | Peroxiredoxin-5 | −0.2214 |
| 15 | CTSD | Cathepsin D | −0.1634 |
| 16 | CTSL2 | Cathepsin V | 0.1167 |
| 17 | APOA1 | Apo A-I | 0.2249 |
| 18 | CXCL13 | BLC | −0.1354 |

TABLE 20
Second Panel of DBV Coefficients

| Index | GeneName | Target | DBV Coefficient |
|---|---|---|---|
| 1 | STC1 | Stanniocalcin-1 | −0.2657 |
| 2 | MMP7 | MMP-7 | −0.2111 |
| 3 | COL18A1 | Endostatin | −0.2376 |
| 4 | HP | Haptoglobin, Mixed Type | −0.0957 |
| 5 | CNTN1 | contactin-1 | 0.2417 |
| 6 | GSN | Gelsolin | 0.2499 |
| 7 | ESM1 | Endocan | −0.1636 |
| 8 | VEGFA | VEGF | −0.2570 |
| 9 | F9 | Coagulation Factor IX | −0.1654 |
| 10 | SLPI | SLPI | −0.2068 |
| 11 | SAA1 | SAA | −0.2395 |
| 12 | CFI | Factor I | −0.1793 |
| 13 | CCL18 | PARC | −0.2122 |
| 14 | LDHB | LDH-H 1 | 0.1707 |
| 15 | GOT1 | GOT1 | −0.2301 |
| 16 | CDON | CDON | 0.2224 |
| 17 | INSR | IR | −0.1370 |
| 18 | SELL | sL-Selectin | 0.1795 |
| 19 | HAMP | LEAP-1 | −0.1923 |
| 20 | NTRK2 | TrkB | 0.1502 |
| 21 | PRDX5 | Peroxiredoxin-5 | −0.1760 |
| 22 | CTSD | Cathepsin D | −0.1436 |
| 23 | IL19 | IL-19 | 0.1204 |
| 24 | CCL3 | MIP-1a | −0.1704 |
| 25 | IL1R1 | IL-1 sRI | −0.1665 |
| 26 | CXCL13 | BLC | −0.1262 |
| 27 | TG | Thyroglobulin | 0.1415 |

What is claimed is:

1. A method of evaluating an individual for renal cell carcinoma (RCC) comprising measuring in a biological sample from the individual the level of at least one biomarker selected from the group consisting of CXCL13, RARRES2, HBA1-HBB and THSB4 wherein said evaluating comprises a determination of no evidence of disease (NED) or no RCC where there is substantially no differential expression of the at least one biomarker relative to a control population, or a diagnosis of evidence of disease (EVD) and RCC when there is a substantial differential expression of the at least one biomarker relative to the control population.

2. The method of claim 1, wherein at least one additional biomarker is measured, wherein the at least one additional biomarker is selected from the group consisting of STC1, KLK3.SerpinA3, COL18A1, AHSG, CNTN1 and MMP7.

3. The method of claim 1, wherein the evaluating of an individual for RCC further comprises the step of combining biomarker detection with additional biomedical information.

4. The method of claim 1, wherein said evaluating comprises any of Stages I-IV of the RCC.

5. The method of claim 1, wherein said evaluating comprises determining a prognosis comprising detecting no evidence of disease (NED) and a prediction of no RCC, or detecting evidence of disease (EVD) and a prognosis for occurrence of RCC.

6. The method of claim 5 wherein said method comprises determining a prognosis of a future course of the RCC.

7. The method of claim 6, wherein the step of assaying a biological sample in step a) occurs at a first time point, and where there is the prognosis of RCC occurrence in step c), the occurrence is predicted to occur at a second time point.

8. The method of claim 1, wherein said evaluating further comprises determining a RCC disease burden in an individual, comprising:
a) selecting a RCC disease burden vector (DBV) modeled on biomarkers that correlate with RCC stage;
b) providing an individual's sample suspected of containing said biomarkers;
c) applying the DBV to the sample biomarkers to determine the individual's disease burden vector score (DBV score);
d) determining the disease burden on the basis of the DBV score.

9. The method of claim 8, wherein the determination of the disease burden in step d) further comprises the step of including in said determination, additional biomedical information.

10. The method of claim 8, wherein the DBV score corresponds to any of RCC stages I-IV or absence of RCC.

11. The method of claim 1 wherein said evaluating comprises determining the recurrence of RCC in an individual who had apparently been cured of RCC, wherein the determining of recurrence comprises a first determination of no evidence of disease (NED) and no recurrence of RCC, or a second determination of EVD and recurrence of RCC.

12. The method of claim 11, wherein the determination of recurrence of RCC further comprises the steps of
repeating the determination of recurrence at pre-determined time points to monitor the individual's response to a therapeutic agent or other treatment.

13. A computer-implemented method for classifying an individual as either having a first evaluation of NED, or as having a second evaluation of EVD, said method comprising:
a) retrieving on a computer biomarker information for an individual, wherein the biomarker information comprises a biomarker value that corresponds to at least one biomarker of Table 1;
b) comparing said biomarker value of step a) to a biomarker value of a control population to determine if there is differential expression; and
c) classifying the individual as having a first evaluation of NED when there is no differential expression in step b) of the biomarker value relative to said control population, and as having a second evaluation of EVD when there is differential expression of the biomarker value relative to the control population.

14. The method of claim 13, wherein said evaluation comprises a determination of diagnosis, determination of prognosis, determination of recurrence of RCC, and/or a combination thereof.

15. The method of claim 14, wherein said evaluation of NED can be indicative of a determination of no diagnosis of RCC, a determination of outcome prediction of no RCC at a selected future time point, a determination of the existence of no RCC disease burden, a determination of no recurrence of RCC, and/or a combination thereof.

16. The method of claim 14, wherein said evaluation of EVD can be indicative of a diagnosis of RCC, a prognosis of an outcome of RCC at a selected future time point, a determination of the existence of a RCC disease burden, a determination of recurrence of RCC, and/or a combination thereof.

17. A computer program product comprising a computer readable medium embodying program code executable by a processor of a computing device or system, the program code comprising:
a) code that retrieves data attributed to a biological sample from an individual, wherein the data comprises a biomarker value that corresponds to the at least one biomarker of Table 1;
b) code for comparing the biomarker value of step a) to a biomarker value of a control population; and
c) code that executes a classification method that indicates a first evaluation of NED when there is no differential expression of the individual's biomarker value in step b) relative to the control population, or second evaluation of EVD when there is differential expression of the individual's biomarker value relative to the control population.

18. A kit useful in detecting one or more biomarkers of Table 1, comprising
a) one or more capture reagents for detecting one or more biomarkers in a biological sample, wherein the biomarkers comprise any of the biomarkers of Table 1; and
b) signal generating material.

19. The kit of claim 18, wherein said capture reagent is immobilized on a solid support.

20. The kit of claim 18, further comprising
c) a software or computer program product for classifying the individual from whom the biological sample was obtained for evaluation of RCC status.
